SEQUENCE LISTING (10 sequences)

SEQ ID NO: 1 - DNA, 562 bp, synthetic construct
  misc_feature 1..562: note = synthetic construct
  source 1..562: mol_type = other DNA
acatgatcaagtagttgtttaacctcgctctgtaggctttgtgtagtatcttctggcttaggtgctgcgccattagtgcggcgaacagcttgaccattagtgcgggaagatttgagacttttagctgtggaagtaggttgaggttgagaagaaccgccgccccaattttgaacagccgtcgccacatcaatacccgtagtttggcgcaactcttccaaaaacgccgccattttagaagctgtattccctttattagcatcaacaatagtcacattatcaacagtaatttcaggaacagtagaagccatagtttttagtaagacttccaacttctgataaaggaaaatttccctagcattaggaccggctgctttccaagactcagccagtcgtcgtgtcccttcagcttgtgctttcccatcttcaataatttgggcagcatccccccgcgccctagcgatcgcctttttacattccgcctctgctggtgcgaccacatcagcttgtagttgctgttctacctgcttaatcctttctttttgaacagagacctcagcctgag

SEQ ID NO: 2 - DNA, 898 bp, synthetic construct
  misc_feature 1..898: note = synthetic construct
  source 1..898: mol_type = other DNA
ccttaaccccctaagccctatgtctgacacccagtcaccccaaccgccttagtcggtattccccactgggtgggcgatacacgatttcccggaaaaattgaaaaaaaattttgtggcaggtgatatgatttgcgatcgaggtgggtaagttagaactacagaaaacaaataacctcatttcaccccgaggagaacgaattatgaaaactgaacttaaagccaaattcctgcaacatatctctcagaaaaaaagagacggtggtttcaccttaatcgaactgttagttgtaattatcattatcggtattttgtcggcgatcgctctgccttccttcttaaaccaagctaacaaagccagggagtccgaagctaaaacatacgttggttcaatcaaccgggctcaacaagcgcgtttgatggaaactggcgagtttactacctcaataactcaactgggtctgggtgtcagcagcgcaacagcaaactacacttataatctactcgctaatgcttctggtgcggtggttaatgccaacggtatagctaatctccgcgactatggtggtctggtctgggtaggtactacagcaggaggggagcgtaccactttggctaaactgtgcgaagcgcctgatgcagaaaccgaggcaaagtttgcagcgggcgctaatagttgtggtacttttagagaaatttaacagataatctagcagtttaggtttcagatggagtcagctagggtctttaaccccatcgtcaactataagatggagagatggcatacggtgacactgttaaagccataactatattgctcttgttgataaaatcagaaactgaggccacttgagagtattgtaaataggtagtacagtagtgctacctattttgttttaaa

SEQ ID NO: 3 - DNA, 972 bp, synthetic construct
  misc_feature 1..972: note = synthetic construct
  source 1..972: mol_type = other DNA
tctggcccttaattgtagtcgatcgcccagaatattataccatccctttaggcgtggcaaatctagccggaactttttctctcgattggcggatagtcgccgccggttccgtcatttctatcgctcctgtgttactactatttttaatcgtacaacggtatattgtgcctacagatacagctacgggggttaaaggttgaggattattaatctcagtcctaataatcaaaatcatatccaccaggcggcgactttattagtcgcagaatttcgggaaaattggccgaatgcttggccgacttatgaacgtggtttagccgaggttatggagtcttttggcgacgatagagttaatctagtcgccgtcgatgaaaatgataatcttctaggctggattggtggtattagccaatatcaaggccatgtttgggaattacatcctttggtagtcaaaagcgattatcaagggttaggaatagggcgaaaattggtcgccaatttagaggattatgtccgctcacaaggagggttaaccttatggttgggtaccgatgacgaaaataatttaacctccctatctggtgtcgaattatacccccattttttggaaaatattgctaacataaaaaaccatggtcgtcatccctatgaattttatcagaaatgcggttttgtgattatgggagtagtccccgatgctaacggtatcgggaaacctgatattttgatggctaaatctctgagaattgacaatcatcctaaatccaactgaggaatcatgaatcatcccacaactaagcatcaaaaaattacatcctgttactggggaatttctgaactacctgggttagatacaggtaatattaaactcctgaatcaatgtggtattgaaaacacccagcaactgctcacgcgcgggagtaataatgctaataaaatcgccttatctaaccaactaggtatcaacatcag

SEQ ID NO: 4 - DNA, 1054 bp, synthetic construct
  misc_feature 1..1054: note = synthetic construct
  source 1..1054: mol_type = other DNA
gtatcagcgttaactatggtgcaatttatcccttgctgaaacgtttggaagaacggggatatattcaagttatcctagaagaggcaggggacgcgggttcgagtcggaaggtttatggtattaccgccgccggacgtgagcgatggttatacaaaatgctagaacagccgcaagagagttgggttaaaagccgttctaggtttctaattaagtatttcttttttagtgacttaaatatacacaatcgcctgaaattgttagaatatcgcctcagagcttgtcagcaaaggatggcatatctgaaccagaaagaggttgaatatgtgccgattgattcctatcaatcagatatctggcaaagggcgaagtttttgttagattcagagtctaaatggctcgaaaaaacctacgataatttgcaacaagaactagggggtggtgattctctatcttcagacagatcacaattgtgaatagccgagatctacaatggcagtagactagggaattgacagtaaaattttgccgccaacaattataatcatactacaatttattatgtcttatccatcatcaacagaatcatcaaactcgaattatcatcagtcggtcgtggaagttgaggggagtggtgaacattcgccaatagaaaaccaaaatcagatagaaagcgatcgccattcggaatcagaacctagtaataaatccaacaattgggtatcagcgaccgtaggaatattactagccctaggagtaggctttgctgggggtcagtggtggtactccagtaaagcgcaagcggacactaccccagcggcggcgagtaattccccccaaagaccgaggggagttccggtgagagtcaccagcatagaaactaggaactttcaggatgctggggaatttgtggggactctggaagcgcgggagtcggtggaagtaagatcggaaatagaagggagaatcacgcaagtatatgttaggtcaggggagatggtaaatcaagggcaggcgatcgcccgtttaagaagtgatgacctagaagcgcaactac

SEQ ID NO: 5 - DNA, 806 bp, synthetic construct
  misc_feature 1..806: note = synthetic construct
  source 1..806: mol_type = other DNA
ggctttttgcgatcgcatcttttgccgtcggtgctttcgccattttaccctatttggtgtggcgaaaacctaaccccgaattttctggggttcccaatctcttcatccgcatttgggatagcaaattcacagctttagtcttaatgttaggggcgatcgtcctagttttttatggtatcactcagggagattggcgtgactttatctatcaatggcaaaatagccgttttattcacgttatgagcctcgacttctgtttattatcagtattatttcccgccttattaggggatgatatcgcgcggcgcggcaaccaggctgcgacttggttatggttggtgacactcattcccctattcggacctttactatatctctgtgtgcgctctcccctgtgcgatcgtctttgagtcaccatccgtagttttcatcaaagcctcattaattcagttagaaaaccgcaagaatccgcaggcagtagtgtaaaatctcaataataggtatcagggtcaacccaagagttaggctaaaattcacctccaaacctgatacaatctcctcatcaaattgcgggatcgtcctatgaacaaaacactatggaaaactagcgcatctctaccgctgctattattatttgtactcatcaatgcttgttcagcagaacctcaagccattggtcgcccacccgtccccgtcaagatagaaaacgtagatactaccgatgttcaatccagcagcgaatataatgccagggttgaaggtatcgaaaactctatcatcagaccccgagttagtggattagtaaa

SEQ ID NO: 6 - DNA, 968 bp, synthetic construct
  misc_feature 1..968: note = synthetic construct
  source 1..968: mol_type = other DNA
gtttgccgcattttaattaatcagcaaacccatgaacaagtcagctatcaatgtctaactgaatatatcggtagcattcacctcaaaggtcgcactcaacctattaatgtttatcaaattaacaatccttagtctgtattaaatctttccatctattctgtcaactgtcctaggtagttatgaatagccacagcttttgatgaatctcctcgcccccagatgtaattcacggcggagacagctccgcacccccacccacattatccccccctcccagagggggacaagataaggagtttcatgattcaccgcgacaccacattaactttatcgccatctttaagataacttgcaccagcgatcgcaatgcgatcgcctacatttaaaccctgtataatttctatactcgcctccgataaatcgcctgttaaacggccttgaagtcttcccacttccacagttcgcgcttccaccgtatcatcaggtaatagacgataaacaatagaactaccgtcagcctggggaacgatggccctagcgggaaccttcattccttgtacagtagtagtagtaatcgccgcccgcagaaacattcccggacgcaagaaagaatcacgaatagcccccaagggaggcaggtcaattttcacagtagcttgtcggctttgaggatcaatcagaggcgcaatttctcgcaccacccctcgcacctgcaaattttgatcagcatcagaagtcacagtaacagcagcaccaattctcacctgtgataactgagtttctggaaccttcgcgtgtagttctaactgattttccgcaataatcgaaaacagttgttgattaccagtaacatcccccactcgcgctgtccgttgggcgacaatgccactcataggcgcacggactacagtttgttcccgttgagtttccaactggcgaacccgcgcctgggcgctacgcacctcagc

SEQ ID NO: 7 - DNA, 1101 bp, synthetic construct
  misc_feature 1..1101: note = synthetic construct
  source 1..1101: mol_type = other DNA
atgaaatgtgaaggttctccggtcgtagaaacacacaattgggatattcctgctgtagcgattgcatgcaaaacctatttagataagacgatgctgcaagatatttctacggcggctgaacaactcaagtataaaaatcccaatgctatgtatattgtagtggcagaatggctcaaacttactgactcggtgaatctgagaaaatataaaattgatcaaatttatattttgcgaaaacaaaaaaatactgatagagaatttagatatcttgagagttatcagaaaaatcctatatactctgatgtagtacaacatctatttctgaaagtacgagagtttctgatttccgactgggagggaggaatatcagatggtttaaatcgtggttttttactctaaaatcttggctatgacaattcctctggcttccccgagtccctgccctaattatcccatatcattattaaactcgcaaggaggtgaatctttccgcgtttgtatgctatgatatgctacaattcagactatacttttctcccttgacaacccaaaccgattagattagatgactgtaaattaatcaataaattatggctaaaaatagttttaatctttccgaatggtcgattcgacaacctgttcccactttggtgctgtttttgatcctgacgctggtggggttgatgtctttttttcaactgggaattgatgatactcctaatatagatattccggcggttacggtgacagttacacaaccgggtgcgggtcctaatgaattggaaaatcaggtgacaaaacaggtcgaagatgcgatcgccggattaggaaatattgaccaacttacctctacagtcagagacggagtatctaccactaccgtgagttttgttttgggaaccgatagcgatcgcgctactaatgatgtccgtaatgcgatcgctcaaattcgccaaagcctaccccaagacattaacgatcctattgtacagcgtctgcgatttgcgggcgggtcgattatgacctatgcggtaacatcacccacccgctcagtagaagaacttagcgatcttgtcgatcgcactattagccgcgccttac

SEQ ID NO: 8 - DNA, 646 bp, synthetic construct
  misc_feature 1..646: note = synthetic construct
  source 1..646: mol_type = other DNA
ctttgagccctggcgcattccccaggatctatgctttgaccacgatcgcattttacaagactatctgcaatttcgccattttggactccgacccaggctaatgtgagtcagtgaacacccgactacccccaactcgcttcttctcagaaaaagagagtaagagacagaggtggcccactaagtatgggggtaatatccacttactcagcaccggaattgggttctaagccccactgatacttgagaaagtgctgatacatattctccctaaccaacatttgttcgtaaagatcaaccaaaaactcctgggcttgttcccggctcatttggtttacctgcattctaaaggaggacaggttaaattgttgttccaaagataagctactagattccatgatgacgtgaactccttaaaagtgaatttgggttaattttctaggatggccacccttgtcccggaaattattacgaattataacaattatttgacaaagtgcctaccttaatatacccaaatctttggatttaccataacagatctagccctaaaatttgtcatccacgatacgagatgactggatagttgggaaaaaactatggctgtggggggataagtggcaataattaaccgaaactgagaggatca

SEQ ID NO: 9 - DNA, 583 bp, synthetic construct
  misc_feature 1..583: note = synthetic construct
  source 1..583: mol_type = other DNA
gtagcaagcgttttggctacctggtcgagtttagcgatactcatagtcttttccaaaatccccaacaagaagccacacaacaatatgttagtggtcgctttggttaagcataacctattcccaagaggtcagaggctacttaacttaatcagccctttctgacctccctctggttgtggtgattggttgatggtgttttgaggctatgcgcctttcatccagtttttcagtaaattatccttgaccatcccttggcgaaatacctcaattaaatactcctgagcttcttctaaacttaggtgtttgacttgttcctggagaaccttgagcttgaattgttgctctatgcttagatttcctggggtttgcattgttcgctcctcctctgaaattctaggggtgagagtacggttttgggtcaaaccgtaacttttgtttacctggaatgtaaattatcgtaacatacacttgacaatctgtccatgatggacatttaggcactaatctatttaacaaaactttggattttccgatctgatagccacagactcagccctaactgccataaaataaacggatagcc

SEQ ID NO: 10 - DNA, 2368 bp, synthetic construct
  misc_feature 1..2368: note = synthetic construct
  source 1..2368: mol_type = other DNA
gatgctaatatgttagcaaaatttcggctccctgtcaacccctgcagccgggcggatttgcgtgttgacgaaaaaatcaaaaaagtgatcgggtgcgtcatagcaattaataatgatttcccgatggggaaatgagcggactgacgcaccaaaattgtggttgagaaattcccggcttaattaaccaggaagttgctcaactaagcgccaccgcgcagcttttcgagaacggtgcgatcttggagagtggaagtatccccagaaatctcctgacccgaagcaagcgatcgcaataaacgccgcataattttacccgaacgagtcttaggcaaatcatcagaaaagcgaatttccccgggacgcgccaaggctccaatttcattaacaacgtgctgcttcaactctttttccaaggcttcatcaggttggcgatcgccttcaagagtaacaaaagccacaatttcctcacccttgacttcatcaggacggccaaccaccgccgcctcagccacagccggatgagaaaccaaagccgactcaacctccatagtccccaagcgatgaccagaaacattaatcacatcatcaacccggcccatcacccagaaatagccatcctcatctcgatgagcgccatcaccagcgaagtagacataatcgccatttttaggtcgtagatactcccaataagtacggcggaagcgctccgggtcgccataaacagtccgcatcatacccggccacgggtgacgcaccaccaaatatcccccagaattattagtaacaggttccccttccgtatcaaccacatcagcaataattccagggaaaggcaaagtagccgaacccggcttagtaggagtcgcaccaggaagaggagtaatcataaaaccccccgtttccgtttgccaccaagtatcaacaatgggacattttccttgaccaatgacgcggttataccaaatccaggcttctgggttaatcggttctcccaccgtacccaaaatccgcaacgaagacaaatcacgagcattagggtgtctttcacccattttcatcaaagctcgaatagcagtaggagcagtatagaaaatagtcacgccatacttttctaccacatcccacagacaaccgggattagaagcacggggagccccttcatacatcaaagttgtcgccccattggacaaaggcccatagacaatataactatggccagtaatccaacccacatcagcggtacaccagtaaacatcagtatcctgtaaatcaaaagcccactgattagtaatgtgactgtaaagattatagccaccagtagtgtgtacaactcccttgggtttgccagtagtcccgctagtgtaaaggataaacagcatatcctcgctatccatttcttcggcgggacattgcccggaagcattttgttgcaagtcatgccaccagtggtcccgtccgggttccatctggattttttgctcagtgcgctgtacgaccaaaacgttatcaacactaggcgcaccagcttgtaaagccttatctacctgttcttttaggggtacgatcgcatctttgcgccaaccaccatcagcagtaatcaccaatttagcttcggcatcttccaggcggtcctttagagcctcggcactgaaaccgccaaaaatgacggtatggggtgcgccaatcctcgcacaagccaacatggcgatcgccgcctctggtatcatgggcatataaatcccaacgcgatcgccttttttgactcccaactgtttaatcacatttgccatttggcaaacttcccgatgtagttgggaataggtcagagtccgagagtctcccggttccccttcccaaattaaagccgccttatttttgcgccaggtagtcaaatggcgatcgaggcaattataggaaatattaattttgccccccacaaaccacttcgcaaaaggtggctgccagtccagcacctgatcccattttttaaaccagtgcaactcattttccgctaattctgcccaaaacgcttccgggttagctttggctttctcgtagagttcccggtactcttccatgctcttaatccgggcctgtttggagaagtcctcggacggattaaataaccgtttctcgtgtaaaattgattcgatcgtcggttgtgacatagtgtgatggtaactttaactacaaagtgaagccattataaccattttaggcctagattaaactctaagaatctttacttttttttgagcagactgctaactagctaactcctccccatgtcaaaccgattaaaatcagttgtcaacttctaagtaactatgcctcctcaactcattatccgtcctgctactgttgatga

(19) United States
(12) Patent Application Publication
(10) Pub. No.: US 2024/0002481 A1
(43) Pub. Date: Jan. 4, 2024

(54) EXPRESSION AND MANUFACTURING OF PROTEIN THERAPEUTICS IN SPIRULINA

(71) Applicant: Lumen Bioscience, Inc., Seattle, WA (US)

(72) Inventors: James Michael ROBERTS, Seattle, WA (US); Hui Zhao, Seattle, WA (US)

(21) Appl. No.: 18/355,696

(22) Filed: Jul. 20, 2023

Related U.S. Application Data
(63) Continuation of application No. PCT/US2022/013529, filed on Jan. 24, 2022.
(60) Provisional application No. 63/140,577, filed on Jan. 22, 2021.

(51) Int. Cl. C07K 16/12; C12N 1/12; C12N 1/20; C12N 15/90; C12P 21/02; A61P 31/04

(52) U.S. Cl. CPC C07K 16/121; C12N 1/12; C12N 1/20; C12N 15/902; C12P 21/02; A61P 31/04; C07K 2317/569; C07K 2317/22

(57) ABSTRACT

The present disclosure provides a method of transforming a population of spirulina cells comprising growing the spirulina cells with: (a) a co-culturing microorganism to induce competence; and (b) a transforming molecule. Further provided are recombinant spirulina cells and pharmaceutical compositions produced by the method of transformation.

### CROSS REFERENCE TO RELATED APPLICATIONS

This Application is a continuation of International Application No.: PCT/US2022/013529, filed Jan. 24, 2022, which claims priority to U.S. Provisional Patent Application No. 63/140,577 filed Jan. 22, 2021, the entire contents of each of which are incorporated by reference herein

### DESCRIPTION OF THE TEXT FILE SUBMITTED ELECTRONICALLY

The contents of the electronic sequence listing (LUBI_028_01US_SeqList_ST26.xml; Size: 23,280 bytes; and Date of Creation: Jul. 17, 2023) are herein incorporated by reference in its entirety.

### BACKGROUND

Arthrospira platensis (commonly known as spirulina) is a photosynthetic cyanobacterium. It is a highly nutritious food that has been consumed for decades in the US, and even longer by indigenous cultures. Its widespread use as a safe food source and proven scalability have driven frequent attempts to convert it into a biomanufacturing platform. But these have been frustrated by spirulina's genetic intractability.

The first successful spirulina transformation protocol that incorporated a mutation in the spirulina genome is disclosed in U.S. Pat. No. 10,131,870, the contents of which are incorporated herein by reference for all purposes. Prior to this disclosure, it was notoriously difficult to stably introduce foreign DNA into spirulina. For example, Zhang (2001) teaches a spirulina transformation using axenic spirulina cells, yet shows no evidence of stably incorporating a mutation in the spirulina genome. Toyomizu et al. (2001) attempted transformation using electroporation and Kawata et al. (2004) attempted transformation using a natural Tn5 transposon, transposase, and cation liposome complex by electroporation. Neither of these approaches resulted in a stable spirulina transformant. Other approaches to create stable mutations in the spirulina genome using chemicals have also failed. For instance, Fang (2013) used ARTP, a method that uses active chemical species at a high concentration to penetrate the cell membrane and damage the cell's DNA, causing random mutations and Yuanyuan (2013) teaches using ethylmethane sulfonate (EMS) which also causes random mutations. Neither of these approaches led to stable spirulina transformants.

Based on the foregoing, there is a need for a reliable system for efficiently transferring exogenous sequences into spirulina as prior attempts have had limited results thereby leaving a need in the art for improved methods of successfully engineering spirulina to express exogenous sequences. Accordingly, provided are novel compositions and methods of using the same to engineer spirulina by way of a natural method comprising at least a non-axenic culture system. Also provided, is a natural method, non-electroporative, of engineering spirulina that at least confers increased transformation efficiency as compared to methods comprising electroporation.

### BRIEF SUMMARY

Until the present disclosure, there has not been a spirulina transformation protocol that induces competence using a co-culturing microorganism. The present disclosure provides a method for transforming spirulina using a co-culturing microorganism to induce competence.

The present disclosure provides a novel efficient and versatile genetic engineering methodology for spirulina and demonstrate that genetically modified spirulina can stably express bioactive protein therapeutics at high levels. The integrated development and manufacturing spirulina platform blends the safety of food-based biotechnology with the ease of genetic manipulation, rapid growth rates and high productivity characteristic of microbial platforms. These features combine for exceptionally low-cost production of biopharmaceuticals to address medical needs that are unfeasible with current biotechnology platforms.

Provided herein is a method of transforming a population of spirulina cells comprising growing the spirulina cells with: (a) a co-culturing microorganism to induce competence; and (b) a transforming molecule. In some embodiments, the co-culturing microorganism is gram-negative. In some embodiments, the co-culturing microorganism is gram-positive. In some embodiments, the co-culturing microorganism is aerobic. In some embodiments, the co-culturing microorganism belongs to the genus Sphingomonas. In some embodiments, the co-culturing microorganism is selected from: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus and Oscillatoria, Koinonema, Oxynema, Planktothrix, and Microcystis. In some embodiments, the co-culturing microorganism belongs to the genus Microcella. In some embodiments, the co-culturing microorganism is selected from M alkaliphile, and M. putealis. In some embodiments, transformation of the spirulina with the transforming molecule deletes one or more genes, loci, or sequences in the spirulina genome. In some embodiments, transformation of the spirulina with the transforming molecule adds one or more genes, loci, or sequences to the spirulina genome. In some embodiments, transformation of the spirulina with the transforming molecule replaces one or more genes, loci, or sequences in the spirulina genome with the transforming molecule. In some embodiments, the spirulina cell is transformed with multiple transforming molecules. In some embodiments, the spirulina is transformed with different transforming molecules in multiple rounds of transformation. In some embodiments, the spirulina is transformed with at least 2 different transforming molecules in 2 rounds of transformation. In some embodiments, the first transformation inserts one transforming molecule into the spirulina genome and the second transformation replaces the first transforming molecule with a different transforming molecule inserted into the spirulina genome. In some embodiments, the transforming molecule is a polynucleotide. In some embodiments, the polynucleotide is DNA. In some embodiments, the DNA is cDNA. In some embodiments, the polynucleotide is comprised in a vector. In some embodiments, the vector is a circular vector. In some embodiments, the vector is linearized. In some embodiments, the polynucleotide is a liner polynucleotide. In some embodiments, the transforming molecule contains one or more homology arms. In some embodiments, the one or more homology arms flank a sequence to be inserted into the spirulina genome. In some embodiments, the homology arm is between about 1000 and about 1500 nucleotides long. In some embodiments, the polynucleotide comprises one or more promoters, terminators, or enhancer sequences. In some embodiments, the promoter is selected from an inducible promoter, a constitutive promoter, and a strong promoter. In some embodiments, the recombinant spirulina express one or more polypeptides or fragments thereof. In some embodiments, the polypeptide is an antibody or fragment thereof. In some embodiments, the antibody or fragment thereof is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof. In some embodiments, the antibody is a VHH antibody. In some embodiments, the polypeptide or fragment thereof is a therapeutic or prophylactic polypeptide. In some embodiments, the therapeutic or prophylactic polypeptide is intended for delivery to the gastrointestinal tract of a subject. In some embodiments, the therapeutic or prophylactic molecule is intended for systemic delivery in a subject. In some embodiments, the therapeutic or prophylactic polypeptide is an endogenous spirulina polypeptide. In some embodiments, the endogenous spirulina polypeptide is found in higher concentrations than found in naturally-occurring spirulina. In some embodiments, the therapeutic or prophylactic polypeptide is exogenous to spirulina. In some embodiments, the exogenous polypeptide is naturally produced by a different bacteria or plant. In some embodiments, the exogenous polypeptide is selected from the group consisting of: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin. In some embodiments, the exogenous polypeptide is an antigen or epitope. In some embodiments, the antigen or epitope is derived from an infectious microorganism, a tumor antigen or a self-antigen associated with an autoimmune disease. In some embodiments, the exogenous polypeptide or a fragment thereof is in a fusion protein. In some embodiments, the spirulina is transformed with a nucleic acid, and wherein at least 2, at least 3, at least 4, or at least 5 copies of a nucleic acid sequence encoding the at least one exogenous polypeptide or fragment thereof are present in the recombinant spirulina. In some embodiments, the spirulina is transformed with a nucleic acid, and wherein 2, 3, 4, 5, 6, 8, 10, 15, 20, 25, 30, 40, or 50 copies of a nucleic acid sequence encoding the at least one exogenous polypeptide or fragment thereof are present in the recombinant spirulina. In some embodiments, the recombinant spirulina comprises at least 2, at least 3, at least 4, or at least 5 different exogenous polypeptides or fragments thereof. In some embodiments, the fusion protein comprises a carrier protein. In some embodiments, the carrier protein is selected from the group consisting of: maltose binding protein, hedgehog hepatitis virus-like particle, thioredoxin, and phycocyanin. In some embodiments, the fusion protein comprises a scaffold protein. In some embodiments, the at least one exogenous polypeptide is linked to a scaffold protein at the N-terminus or the C-terminus, or in the body of the scaffold protein. In some embodiments, the scaffold protein is selected from the oligomerization domain of C4b-binding protein (C4BP), cholera toxin b subunit, or oligomerization domains of extracellular matrix proteins. In some embodiments, the at least one exogenous polypeptide and the scaffold protein are separated by about 1 to about 50 amino acids. In some embodiments, the fusion protein comprises multiple copies of the at least one exogenous polypeptide or fragment thereof, wherein the at least one exogenous polypeptide or fragment thereof and the scaffold protein are arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the at least one exogenous polypeptide or fragment thereof, SP is the scaffold protein, n, n1, and n2 represent the number of copies of the at least one exogenous polypeptide or fragment thereof. In some embodiments, the therapeutic or prophylactic molecule is monomeric. In some embodiments, the therapeutic or prophylactic molecule is multimeric. In some embodiments, the therapeutic or prophylactic molecule is trimeric. In some embodiments, the multimer is heteromeric. In some embodiments, the multimer is homomeric. In some embodiments, the multimer is arranged in a nanoparticle. In some embodiments, the spirulina is selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In some embodiments, transformation is achieved by growing the spirulina, co-culturing microorganism, and transforming molecule in liquid culture for between 1 and 4 weeks. In some embodiments, the co-culture is grown in liquid culture for at least 2 weeks. In some embodiments, the co-culture is grown in liquid culture for at least 3 weeks.

Provided herein is a culture of a population of recombinant spirulina cells created by a method provided herein.

Provided herein is a recombinant spirulina cell created by a method of the disclosure.

Provided herein is a method of transforming a population of spirulina cells comprising growing the spirulina cells (a) under conditions that induce competence, and (b) with a transforming molecule.

Provided herein is a composition that comprises (a) a population of spirulina cells; (b) at least a portion of a co-culturing microorganism in an amount effective to induce competence; and (c) a transforming molecule. In some embodiments, the transforming molecule comprises a polynucleotide. In some embodiments, the polynucleotide comprises DNA. In some embodiments, the DNA is cDNA. In some embodiments, the cDNA comprises at least two sequences encoding a first and a second homology arm, and wherein the first and the second homology arm are between about 1000 and about 1500 nucleotides long. In some embodiments, the first and the second homology arm bind to a Spirulina sequence comprising at least a portion of a GNAT family N-acetyltransferase sequence. In some embodiments, the at least a portion of the co-culturing microorganism comprises the entire microorganism. In some embodiments, the at least a portion of the co-culturing microorganism comprises a portion of a microorganism. In some embodiments, at least about 5% of the spirulina cells in the population are transformed as determined by sequencing. In some embodiments, the first and the second homology arms flank a sequence encoding an antibody or fragment thereof. In some embodiments, the antibody or fragment thereof is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof. In some embodiments, an antibody comprises a VHH antibody. In some embodiments, the VHH antibody binds a target in a gastrointestinal tract. In some embodiments, the target comprises a pathogen or cancer cell. In some embodiments, the pathogen is a bacterium. In some embodiments, the bacterium comprises campylobacter. In some embodiments, the co-culturing microorganism is a bacteria. In some embodiments, the bacteria are gram positive. In some embodiments, the bacteria are gram negative. In some embodiments, the bacteria are of an order selected from the group consisting of: Micrococcales, Xanthomonadales, Purple sulfur bacteria, Nevskiales, Hyphomicrobiales, Mycobacteriales, Bacillales, Nitrosomonadales, Oceanospirillales, Oscillatoriales, and combinations thereof. In some embodiments, the bacteria are of a genus selected from the group consisting of: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Akanivorax, Dyella, Rhodanobacter, Halomonas, Variovorax, Frateuria, Dokdonella, Cupriavidus, Oscillatoria, Koinonema, Oxynema, Planktothrix, and Microcystis. In some embodiments, the bacteria comprise Sphingomonas or Microcella. In some embodiments, when the composition comprises a volume from about 30 to about 40 μL, the composition comprises: (a) about 0.1 to 1 OD of the spirulina cells when measured at 750 nm wavelength as determined by spectrophotometry; and (b) about 275 ng to 325 ng of the transforming molecule.

Provided herein is a pharmaceutical composition generated using a method of the disclosure. In some embodiments, the pharmaceutical is in unit dose form.

Provided herein is a method of treating a disease or disorder in a subject in need thereof, comprising administering a pharmaceutical composition of the disclosure, thereby treating the disease or disorder. In some embodiments, the disease or disorder is of a tract comprising a mucosal membrane. In some embodiments, the tract comprises the gastrointestinal tract. In some embodiments, the disease or disorder comprises an infection of Campylobacter jejuni. In some embodiments, the administering is an oral administration.

Provided herein is a container that comprises a composition of the disclosure. In some embodiments, the container comprises a bioreactor.

Provided herein is a kit that comprises: (a) a composition that comprises spirulina; (b) at least a portion of a co-culturing microorganism; (c) a growth or storage medium; and (d) instructions for use thereof.

In some embodiments, the present disclosure provides a method of transforming a population of spirulina cells comprising growing the spirulina cells with: (a) a co-culturing microorganism to induce competence; and (b) a transforming molecule.

In some embodiments, the co-culturing microorganism is gram-negative. In some embodiments, the co-culturing microorganism is gram-positive. In some embodiments, the co-culturing microorganism is aerobic.

In some embodiments, the co-culturing microorganism belongs to the genus Sphingomonas. In some embodiments, the co-culturing microorganism is selected from: Sphingomonas abaci, S. adhaesiva, S. aerolata, S. aquatilis, S. asaccharolytica, S. aurantiaca, S. azotifigens, S. cloacae, S. dokdonensis, S. echinoides, S. elodea, S. faeni, S. genosp. 1, S. genosp. 2, S. koreensis, S. mall, S. melonis, S. mucosissima, S. oligophenolica, S. panni, S. parapaucimobilis, S. paucimobilis, S. phyllosphaerae, S. pituitosa, S. pruni, S. rhizogenes, S. roseiflava, S. sanguinis, S. suberifaciens, S. taejonensis, S. trueperi, S. ursincola, S. wittichii, S. xenophaga, S. yabuuchiae, S. yunnanensis, and Sphingomonas sp.

In some embodiments, the co-culturing microorganism belongs to the genus Microcella. In some embodiments, the co-culturing microorganism is selected from M alkaliphile, and M putealis.

In some embodiments, transformation of the spirulina or spirulina cell population with the transforming molecule deletes one or more genes, loci, or sequences in the spirulina genome.

In some embodiments, transformation of the spirulina or spirulina cell population with the transforming molecule adds one or more genes, loci, or sequences to the spirulina genome.

In some embodiments, transformation of the spirulina or spirulina cell population with the transforming molecule replaces one or more genes, loci, or sequences in the spirulina genome with the transforming molecule.

In some embodiments, the spirulina cell or spirulina cell population is transformed with multiple transforming molecules. In some embodiments, the spirulina or spirulina cell population is transformed with different transforming molecules in multiple rounds of transformation. In some embodiments, the spirulina or spirulina cell population is transformed with at least 2 different transforming molecules in 2 rounds of transformation. In some embodiments, first transformation inserts one transforming molecule into the spirulina genome and the second transformation replaces the first transforming molecule with a different transforming molecule inserted into the spirulina genome.

In some embodiments, the transforming molecule is a polynucleotide. In some embodiments, the polynucleotide is DNA. In some embodiments, the DNA is cDNA. In some embodiments, the polynucleotide is comprised in a vector. In some embodiments, the vector is a circular vector. In some embodiments, the vector is linearized. In some embodiments, the polynucleotide is a liner polynucleotide.

In some embodiments, the transforming molecule contains one or more homology arms. In some embodiments, the one or more homology arms flank a sequence to be inserted into the spirulina genome. In some embodiments, the homology arm is between 1000 and about 1500 nucleotides long.

In some embodiments, the polynucleotide comprises one or more promoters, terminators, or enhancer sequences. In some embodiments, the promoter is selected from an inducible promoter, a constitutive promoter, and a strong promoter.

In some embodiments, the recombinant spirulina express one or more polypeptides or fragments thereof.

In some embodiments, the polypeptide is an antibody or fragment thereof. In some embodiments, the antibody or fragment thereof is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof. In some embodiments, the antibody is a VHH antibody.

In some embodiments, the polypeptide or fragment thereof is a therapeutic or prophylactic polypeptide. In some embodiments, the therapeutic or prophylactic polypeptide is intended for delivery to the gastrointestinal tract of a subject.

In some embodiments, the therapeutic or prophylactic molecule is intended for systemic delivery in a subject.

In some embodiments, the therapeutic or prophylactic polypeptide is an endogenous spirulina polypeptide. In some embodiments, the endogenous spirulina polypeptide is found in higher concentrations than found in naturally occurring spirulina.

In some embodiments, the therapeutic or prophylactic polypeptide is exogenous to spirulina. In some embodiments, the exogenous polypeptide is naturally produced by a different bacteria or plant. In some embodiments, the exogenous polypeptide is selected from the group consisting of: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin. In some embodiments, the exogenous polypeptide is an antigen or epitope. In some embodiments, the antigen or epitope is derived from an infectious microorganism, a tumor antigen or a self-antigen associated with an autoimmune disease

In some embodiments, the exogenous polypeptide or a fragment thereof is in a fusion protein. In some embodiments, the fusion protein comprises a carrier protein. In some embodiments, the fusion protein comprises multiple copies of the at least one exogenous polypeptide or fragment thereof, wherein the at least one exogenous polypeptide or fragment thereof and the scaffold protein are arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the at least one exogenous polypeptide or fragment thereof, SP is the scaffold protein, n, n1, and n2 represent the number of copies of the at least one exogenous polypeptide or fragment thereof.

In some embodiments, the spirulina is transformed with a polynucleotide, and wherein at least 2, at least 3, at least 4, or at least 5 copies of a nucleic acid sequence encoding the at least one exogenous polypeptide or fragment thereof are present in the recombinant spirulina. In some embodiments, the spirulina is transformed with a nucleic acid, and wherein 2, 3, 4, 5, 6, 8, 10, 15, 20, 25, 30, 40, or 50 copies of a nucleic acid sequence encoding the at least one exogenous polypeptide or fragment thereof are present in the recombinant spirulina. In some embodiments, the recombinant spirulina comprises at least 2, at least 3, at least 4, or at least 5 different exogenous polypeptides or fragments thereof.

In some embodiments, the carrier protein is selected from the group consisting of: maltose binding protein, hedgehog hepatitis virus-like particle, thioredoxin, and phycocyanin. In some embodiments, the fusion protein comprises a scaffold protein. In some embodiments, the at least one exogenous polypeptide is linked to a scaffold protein at the N-terminus or the C-terminus, or in the body of the scaffold protein. In some embodiments, the scaffold protein is selected from the oligomerization domain of C4b-binding protein (C4BP), cholera toxin b subunit, or oligomerization domains of extracellular matrix proteins. In some embodiments, the at least one exogenous polypeptide and the scaffold protein are separated by about 1 to about 50 amino acids.

In some embodiments, the therapeutic or prophylactic molecule is monomeric. In some embodiments, the therapeutic or prophylactic molecule is multimeric. In some embodiments, the therapeutic or prophylactic molecule is trimeric. In some embodiments, the multimer is heteromeric. In some embodiments, the multimer is homomeric. In some embodiments, the multimer is arranged in a nanoparticle.

In some embodiments, the spirulina is selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f acutissima, A. neapolitana, A. nordstedtii, A. oceanica, A. okensis, A. pellucida, A. platensis, A. platensis var. non-constricta, A. platensis f granulate, A. platensis f minor, A. platensis var. tenuis, A. santannae, A. setchellii, A. skujae, A. spirulinoides f tenuis, A. spirulinoides, A. subsalsa, A. subtilissima, A. tenuis, A. tenuissima, and A. versicolor.

In some embodiments, transformation is achieved by growing the spirulina, co-culturing microorganism, and transforming molecule in liquid culture for between 1 and 4 weeks. In some embodiments, the co-culture is grown in liquid culture for at least 2 weeks. In some embodiments, the co-culture is grown in liquid culture for at least 3 weeks.

In some embodiments, the present disclosure provides a culture of a population of recombinant spirulina cells created by the methods disclosed herein.

In some embodiments, the present disclosure provides a recombinant spirulina cell created by the methods disclosed herein.

In some embodiments, the present disclosure provides a method of transforming a population of spirulina cells comprising growing the spirulina cells (a) under conditions that induce competence, and (b) with a transforming molecule.

### BRIEF DESCRIPTION OF THE DRAWINGS

FIG. 1A-D shows a schematic of Homologous recombination of DNA into the spirulina chromosome. Panel A. Illustration of genomic integration. Donor plasmid DNA containing a gene-of-interest (GOI) flanked by left and right homology arms (LHA and RHA respectively) is transformed into spirulina. A double-crossover event allows the GOI to be inserted at the target integration locus. Panel B. Diagram of primer pairs used in PCR to confirm correct genomic integration. The primer pairs for amplification of the LHA and RHA include one priming site (MP1 and MP4) that is only present in the spirulina genome and absent from the donor plasmid. The PCR product of the central primer pair (MP5+MP6) is sequenced by Sanger sequencing to confirm fidelitous integration. Panel C. Segregation analysis of a transgenic spirulina strain (SP607) over several months after transformation. Spirulina was transformed on day 0 with DNA containing an antibiotic marker (Aada) and cultured under spectinomycin selection. PCR products of the full transgene locus (primers MP1 and MP4) were amplified from genomic DNA samples collected at the indicated timepoints. The extent of segregation was assessed by loss of the wild-type loci band (SP3). Complete congregation was observed by day 72. Panel D. Segregation analysis of a transgenic strain (SP79) cultured continuously for more than 3 years. PCR amplification was performed with primers targeting the full transgene locus (primers MP1 and MP4).

FIG. 2A-B shows a schematic for markerless transgene integration. Panel A. Markerless integration of a GOI is achieved by first replacing a native antibiotic resistance gene (KanR) with a non-native marker conferring spectinomycin resistance (AadA). A fully segregated strain containing the AadA marker can then be transformed with a DNA cassette containing the native KanR gene flanked by a transgenic GOI. The final construct contains no non-native resistance genes. Panel B. Integration of the markerless transgene locus is confirmed by PCR analysis and sequencing using the indicated pairs of primers spanning different regions of the integrated locus.

FIG. 3A is a graphic showing multimeric scaffolds with sample expression data for VHHs in spirulina. Monomeric (maltose-binding protein (MBP) and thioredoxin(TRX)), dimeric (5HVZ), trimeric (cTRP), and heptameric (4B0F) scaffolding proteins have been used to multimerize VHHs expressed in spirulina. Inter-subunit disulfides confer additional stability to the dimeric and heptameric scaffolds; these forms were commonly expressed with an MBP tag to improve solubilization. Inset capillary electrophoresis immunoassay (CEIA) results for each scaffold demonstrate spirulina expression of a SARS-CoV2 RBD-binding VHH fused to the indicated scaffolding protein. All VHH fusions were observed at the appropriate size. FIG. 3B shows increase in binding activity by dimerization of VHHs as measured by ELISA with purified VHH. FIG. 3C shows an increase in binding activity by dimerization of VHHs as measured by ELISA with spirulina extract. E. coli-expressed and purified monomeric (PP622) and dimeric (PP661) forms of the RBD-binding VHH were assayed with RBD and compared with the binding activity of identical proteins present in spirulina extracts (SP1464 and SP1477 respectively). The concentration of VHH in the spirulina extracts was determined by CEIA. EC₅₀s of 18.3 nM and 52.3 nM were measured for the monomeric VHH in purified (PP622) and extract (SP1464) samples respectively, while EC₅₀s for the dimeric VHH were 0.32 nM and 1.29 nM for the purified (PP661) and extract forms (SP1477).

FIG. 4A-D show the characterization of spirulina-expressed anti-campylobacter VHH. FIG. 4A shows CEIA quantification of aa682 in SP1182. A standard curve of purified aa682 protein measured on a Jess instrument by anti-His-tag detection using a fluorescent secondary detection antibody in the IR channel is shown. Clarified lysate from spray-dried SP1182 was loaded at a concentration of 0.2 mg biomass/mL. A single peak was observed at the correct molecular weight of 54.8 kD and soluble aa682 was measured at −3% of total dried biomass based using the standard curve. FIG. 4B shows binding kinetics of spirulina-expressed aa682 with recombinant flaA measured by BLI. Streptavidin coated biosensors were loaded with biotinylated flaA and association and dissociation were measured with the indicated concentrations of aa682. Curve fitting was performed using a 1:1 binding model. FIG. 4C shows binding of VHH to intact C. jejuni. Soluble extracts from spray dried spirulina biomass containing an irrelevant VHH (SP257) or an analog of aa682 (SP526) were incubated with C. jejuni 81-176 and stained with a fluorescent anti-His-tag antibody. Fluorescence was measured by flow cytometry. FIG. 4D shows motility inhibition of C. jejuni motility by aa682. Two strains of C. jejuni (81-176 and CG8421) were grown on soft agar plates in the presence of aa682 or an irrelevant VHH control (PP496). Samples were prepared in triplicate and motility halos were measured either 40 h (81-176) or 66 h (CG8421) after plating. Motility halo area was calculated from the halo diameter. Data represented as mean±SD.

FIG. 5A-D shows prevention of C. jejuni infection in mice. FIG. 5A shows shedding of C. jejuni in a mouse model of infection. In a pilot experiment, mice received a daily 200 μL dose spirulina biomass resuspensions or vehicle between days−1 and +3 relative to challenge (5 total doses). Spirulina strain SP227 expressed no VHH and SP526 expressed an analog of aa682. Bacterial shedding in stool was measured 7 days after challenge. FIG. 5B shows biomarkers of inflammation (LCN-2 and MYO) were measured in stool 11 days after infection. Mice received a daily 200 μL dose of spirulina biomass resuspensions or vehicle on days −1, 0, +1 relative to challenge. Spirulina strain SP257 expressed an irrelevant VHH. FIG. 5C shows bacterial shedding after treatment with a single dose of SP526. Mice received a single 400 μL dose of spirulina resuspension or vehicle 1.5 h before challenge with C. jejuni. Bacterial shedding in stool was measured 24 and 72 h after challenge. FIG. 5D shows markers of inflammation (LCN2 and PMNs) were measured 72 h after challenge and treatment with a single dose of SP526. All data represented as mean±SEM.

FIG. 6A-B shows a cost optimization scheme. FIG. 6A shows cost components of cGMP biomass production. FIG. 6B shows spirulina productivity is a function of light intensity and is empirically determined in the described system with SP1182 as the production organism and with current operating parameters. Cost per unit biomass includes labor, capitalized cost of operating lighting system (varies by light intensity), and capitalized costs of other upstream components (independent of light intensity). Minimal cost per unit biomass was achieved at a light intensity of approximately 100 μmol/m₂/sec.

FIG. 7 depicts stability testing of SP1182 binding activity. Batches of spray-dried SP1182 were stored at room temperature for the indicated amount of time and binding activity was assessed by ELISA. Samples were tested in duplicate. Binding of purified aa682 to recombinant flaA was used to generate a standard curve by linear regression. The standard curve was used to calculate the concentration of active aa682 in SP1182 lysates based on ELISA binding activity. VHH activity was normalized to 100% assuming an expression level of 3% aa682 per unit of biomass. Each point represents a different batch of biomass, and data are presented as mean±SD. Red dotted lines indicate upper and lower 95% CI of linear regression analysis of samples.

FIG. 8A-E shows in vitro analyses of protease resistance of aa682 and SP1182 biomass. FIG. 8A shows an SDS-PAGE analysis of purified aa682 incubated with simulated gastric fluid supplemented with 2000 U/mL pepsin. Pepsin band is indicated by loading 20 μg of pepsin. Digestion of aa682 was quenched at intervals ranging from 2 to 120 min. This data is representative of 2 independent experiments. FIG. 8B depicts results of a CEIA of pepsin-digested spirulina biomass resuspension. Dried spirulina biomass of SP1182 was resuspended in simulated gastric buffer and incubated with pepsin for 0 to 120 min or overnight. Whole biomass samples were denatured and analyzed by CEIA. Recombinant aa682 was detected with an anti-His-tag antibody and the data are representative of 4 independent experiments. FIG. 8C shows an immunoassay analysis of spirulina biomass resuspended in low pH gastric simulating buffer conditions. Dried spirulina biomass of SP1182 was resuspended in simulated gastric buffer (pH 3.0, no pepsin) and the presence of aa682 was analyzed in soluble buffer (A). The soluble fraction of biomass subsequently pelleted and resuspended at a higher pH (B) was compared to aa682 extraction by direct resuspension in a high pH bicarbonate buffer (C). The data are representative of 2 independent experiments. D. ELISA-based antigen-binding analysis of spirulina extracts from biomass resuspended in gastric-simulated buffer condition. Soluble protein extracts of (A), (B), and (C) were prepared as in FIG. 8C were assayed for antigen binding to recombinant flaA. Samples (B) and (C) yielded approximate EC₅₀ binding values of 85.8 μg/mL and 29.2 μg/mL of biomass, respectively. Data presented are averages of 2 replicates. FIG. 8E shows ELISA binding activity of aa682 after in vitro protease digestion with intestinal proteases. Lysates from SP1182 were incubated with 0.1 or 0.01 mg/mL of trypsin or chymotrypsin (chymo.) for 1 hr. After protease activity was neutralized, aa682 binding activity to recombinant flaA was measured by ELISA. Bound VHH was detected with an anti-VHH antibody cocktail. Samples were assayed in duplicate.

FIG. 9 shows a table of competence genes. The presence of competence genes in sequenced arthrospira/limnospira genomes was determined using reciprocal best hits against the A. platensis NIES-39 competence genes previously identified in Taton et al., 2020. Genomes were retrieved on GenBank and BLASTp was used to identify reciprocal hits with e-values 50%. Cell labels indicate the percent identity relative to the respective NIES-39 gene; cells in grey indicate that no homolog was identified. Lyngbya aestuarii BL J was included as an outgroup.

FIG. 10A-D are model representations of heterologous proteins designed for expression in spirulina. FIG. 10A is a ribbon representation of a monomeric VHH (orange; PDB ID:6WAQ) with the solubility enhancer, MBP (green; PDB ID: 5M13). The mature, folded protein results in a monomeric VHH as a fusion to MBP and a C-termini 6×-His affinity tag. FIG. 10B is a ribbon representation of a VHH (orange) with a dimerization motif (blue; PDB ID: 5HVZ) and the solubility enhancer, MBP (green). The mature, folded protein results in a dimeric VHH where dimerization is facilitated by the disulfide-linked dimerization motif. The single polypeptide also contains the solubility enhancer MBP and C-terminal 6×-His affinity tag. FIG. 10C is a ribbon representation of a trimeric VHH (orange). The mature, folded protein results in trimeric VHH (orange) where trimerization is facilitated by the self-assembling homotrimer t-cTRP9X3 (blue; Hallinan J., et al. Structures and behavior of de novo designed circular tandem repeat proteins with novel repeat topologies and increased contact surfaces and thickness. In preparation.). The single polypeptide also contains a C-terminal 6×-his affinity tag. FIG. 10D is a ribbon representation of heptameric VHH (orange) with the heptamerization motif (blue; PDB ID: 4B0F). The mature, folded protein results in a heptameric VHH where heptamerization is due to intrachain disulfide bond between individual protomers. The polypeptide also contains an N-terminal solubility enhancer MBP fusion and C-terminal 6×-his affinity tag.

FIG. 11 depicts VHH stability during spray drying. FlaA binding activity of aa682 in biomass versus drying temperature. Biomass was dried across a range of temperatures, extracted at 10 mg/mL biomass, and the extracts were diluted to a constant 0.039 mg/mL assay concentration. Binding activity of the extracts to FlaA was measured by ELISA. Binding activity was unaffected by drying temperatures <73° C.

FIG. 12A is a graphic of a partial structure of spirulina strain SP1182, showing filaments, individual cell, and cytoplasm location. FIG. 12B is a sequence schematic and ribbon diagram of aa682 (LMN-101/SP1182/PP322/FlagV6F23-MBP-H6). FIG. 12C is an annotated image of the LMN-101 sequence. The anti-campylobacter VHH domain is highlighted in gray. CDRs are denoted by boxes, a single disulfide bond is formed between the two cysteines highlighted in yellow, the sequence of the maltose-binding protein domain is highlighted in teal, and a hexahistidine tag is highlighted in magenta. Two short flexible linkers, a G-G and a G-S-G, serve to bridge the VHH and MBP and the MBP and hexahistidine tag respectively.

### DETAILED DESCRIPTION

Though not a true plant, photosynthetic spirulina is the only microorganism that is farmed as a food at commercial scale, world-wide. It has many unique biological traits that differentiate it from existing expression platforms, which include simple, low-cost growth and downstream processing; a photosynthetic metabolism; and the built-in safety afforded by manufacturing and delivering edible protein therapeutics in a food. In addition, its high protein content (50-70% of biomass) exceeds all other staple food crops, making it a strong candidate for the expression of therapeutic proteins. Spirulina's asexual reproduction mitigates the risk of gene escape into the food chain, and the associated food security concerns and regulatory burdens. Spirulina therefore promises all the benefits of plant-based biopharmaceuticals, and—with the discoveries reported here—overcomes the challenges and limitations associated with commercial adoption of other food-based platforms.

The instant disclosure provides a versatile genetic engineering method for spirulina and development of indoor cultivation technology suitable for the large-scale manufacturing of biopharmaceuticals.

### Spirulina

Provided are compositions that comprise spirulina and methods of using the same to engineer the spirulina to express exogenous sequences, such as the transforming molecule of the disclosure. In some embodiments, the spirulina is utilized to generate an exogenous polypeptide or fragment thereof. Accordingly, also provided are compositions that comprise engineered spirulina. In some embodiments, the engineered spirulina expresses a transgene encoded by a transforming molecule.

The methods disclosed herein can be used to transform any spirulina to contain and/or express any extra endogenous molecule or polypeptide, exogenous molecule or polypeptide, transgene, small molecule, promoter, enhancer terminator, marker, antigen, and/or epitope. In some embodiments, compositions comprising spirulina are utilized to manufacture a biopharmaceutical product for therapeutic use.

As used herein “spirulina” is synonymous with “Arthrospira.” In some embodiments, any species of spirulina can be utilized. Spirulina can grow in fresh water or salt water. In some embodiments, spirulina can be acquired from a culture collection source.

Compositions of the present disclosure can comprise any one of the following species of spirulina: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jennerif. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In some embodiments, a species of spirulina comprises Spirulina platensis. In some embodiments, a species of spirulina comprises Arthrospira platensis.

In some embodiments, a composition that comprises spirulina is not axenic. In some embodiments, a composition that comprises spirulina is substantially not axenic. In some embodiments, a spirulina culture is axenic. The status of a culture, such as determining whether it is axenic or not, can be determined by way of microscopic examination and/or confirmed by negative results after cultivation. As described in the current state of the art, axenic cultures have been largely utilized as a medium in which to engineer spirulina. Use of axenic cultures, such as those in which only a single species, variety, or strain of organism is present and entirely free of all other contaminating organisms, are the preferred means of engineering cells as the presence of contaminants may affect growth and/or engineering of the target cell. However, provided herein are compositions and methods that provide surprising results via the use of non-axenic cultures that comprise spirulina. In some embodiments, the non-axenic cultures provided comprise spirulina and a co-culturing microorganism provided herein.

In some embodiments, a culture of spirulina that is not axenic has increased efficiency of transformation as compared to an otherwise comparable culture that is axenic. The increased efficiency of transformation can be at least about or at most about: 0-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold as compared to the otherwise comparable culture that is axenic.

In some embodiments, the present disclosure provides a recombinant spirulina cell created using the methods disclosed herein. In some embodiments, the present disclosure provides a population comprising recombinant spirulina cells created using the methods disclosed herein. In some embodiments, the present disclosure provides a culture of a population of recombinant spirulina cells created using the methods discloses herein and one or more populations of a co-culturing microorganism.

In some embodiments, a population of recombinant spirulina, or a culture of a population of recombinant spirulina cells and one or more populations of a co-culture microorganism, displays greater growth compared to a population of unmodified spirulina or a culture of unmodified spirulina and one or more populations of a co-culture microorganism.

In some embodiments, a population of recombinant spirulina, or a culture of a population of recombinant spirulina cells and one or more populations of a co-culture microorganism, displays increased biomass compared to a population of unmodified spirulina or a culture of unmodified spirulina and one or more populations of a co-culture microorganism.

In some embodiments, a population of recombinant spirulina, or a culture of a population of recombinant spirulina cells and one or more populations of a co-culture microorganism, displays increased photosynthesis compared to a population of unmodified spirulina or a culture of unmodified spirulina and one or more populations of a co-culture microorganism.

In some embodiments, a population of recombinant spirulina, or a culture of a population of recombinant spirulina cells and one or more populations of a co-culture microorganism, displays increased oxygen consumption compared to a population of unmodified spirulina or a culture of unmodified spirulina and one or more populations of a co-culture microorganism.

In some embodiments, a population of recombinant spirulina, or a culture of a population of recombinant spirulina cells and one or more populations of a co-culture microorganism displays increased expression of one or more polypeptides compared to a population of unmodified spirulina or a culture of unmodified spirulina and one or more populations of a co-culture microorganism. In some embodiments, the polypeptide is an exogenous polypeptide. In some embodiments, the polypeptide is an endogenous polypeptide. In some embodiments, the expression of the endogenous polypeptide is increased by adding additional copies of a nucleotide sequence encoding said endogenous polypeptide to the recombinant spirulina. In some embodiments, the expression of the endogenous polypeptide is increased by adding a promoter and/or enhancer to the recombinant spirulina that increases expression of the endogenous polypeptide. In some embodiments, the promoter is a strong promoter, a constitutive promoter, or an inducible promoter. In some embodiments, a promoter is a weak promoter. A weak promoter may be useful in cases where a protein encoded by a transforming molecule is toxic. In some embodiments, expression of an endogenous polypeptide is increased by deleting, disrupting, or downregulating an inhibitor of expression of the endogenous polypeptide.

In some embodiments, a population of recombinant spirulina, or a culture of a population of recombinant spirulina cells and one or more populations of a co-culture microorganism displays increased expression of one or more nucleotides compared to a population of unmodified spirulina or a culture of unmodified spirulina and one or more populations of a co-culture microorganism. In some embodiments, the one or more nucleotides refers to an RNA.

In some embodiments, provided is a composition that comprises spirulina. In some embodiments, the culture comprises at least about or at most about 3%, 5%, 10%, 15%, 20%, 25%, 30%, 35%, 40%, 45%, 50%, 55%, 60%, 65%, 70%, 75%, 80%, 85%, 90%, 95%, or 100% transformed spirulina. In some embodiments, a higher level of transformation is achieved when a non-axenic culture is utilized as compared to the use of an axenic culture. Accordingly, non-axenic cultures are provided for improved methods of engineering spirulina to express exogenous sequences, such as those encoded by subject transforming molecules. In some embodiments, the transformed spirulina comprise one or more antibody or fragments thereof in their genome. In some embodiments, the transformed spirulina comprise one or more disrupted genomic sequences. In some embodiments, the transformed spirulina comprise one or more sequences encoding an antibody or fragment thereof inserted adjacent to or within a genomic sequence. Exemplary antibodies or fragments thereof are described herein.

In some embodiments, a composition that comprises spirulina comprises at least about or at most about: 0 OD, 0.1 OD, 0.2 OD, 0.3 OD, 0.4 OD, 0.5 OD, 0.6 OD, 0.7 OD, 0.8 OD, 0.9 OD, 1 OD, 1.1 OD, 1.2 OD, 1.3 OD, 1.4 OD, 1.5 OD, 1.6 OD, 1.7 OD, 1.8 OD, 1.9 OD, or up to about 2 OD of spirulina as determined by spectrophotometry using a 750 nm wavelength. In some embodiments, a composition comprises from about 0.1 to 1 OD, 0.5 to 1 OD, 0.8 to 1.2 OD, 0.5 to 1.5 OD of spirulina as determined by spectrophotometry using a 750 nm wavelength. In some embodiments, a composition comprises from about 0.1 to 1 OD of spirulina as determined by spectrophotometry using a 750 nm wavelength.

### Co-Culturing Microorganisms or Portions Thereof

In some embodiments, the spirulina is grown in the presence of one or more co-culturing microorganisms or portions thereof. As described herein, use of non-axenic cultures of spirulina surprisingly confer increased efficiency of transformation as compared to otherwise comparable axenic cultures. As used herein, the term co-culturing microorganism can refer to an entire micro-organism or a functional fragment that is effective in inducing competence of spirulina by way of its presence in a culture of spirulina. In some embodiments, a portion of a co-culturing microorganism can be a functional fragment of a microorganism. In some embodiments, a co-culturing microorganism comprises a bacterium, archaea, fungi (yeasts and molds), algae, protozoa, and viruses.

In some embodiments, a co-culturing microorganism comprises a bacterium. In some embodiments, the co-culturing microorganism is gram-negative. In some embodiments, the co-culturing microorganism is gram-positive. In some embodiments, the co-culturing microorganism is aerobic. In some embodiments, the two or more co-culturing microorganisms are of the same genus. In some embodiments, the two or more co-culturing microorganisms are of different genera.

In some embodiments, the co-culturing microorganism is viable under conditions suitable for the growth of spirulina. For example, the co-culturing microorganism may be viable at light levels of between about 500 and about 2500 μmol/m2/sec. In some embodiments, the co-culturing microorganism may be viable at light levels of between about 550-2000 μmol/m2/sec, 600-1500 μmol/m2/sec, 700-1000 μmol/m2/sec, or 800-950 μmol/m2/sec. In some embodiments, a co-culturing microorganism is viable at light levels of about: 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000 μmol/m2/sec.

In some embodiments, the co-culturing microorganism is viable under conditions suitable for the growth of spirulina. For example, the co-culturing microorganism may be viable at about 35° C. In some embodiments, the co-culturing microorganism is viable at a temperature of at least about or at most about: 30° C., 31° C., 32° C., 33° C., 34° C., 35° C., 36° C., 37° C., 38° C., 39° C., or 40° C. In some embodiments, the co-culturing microorganism is viable at a temperature of at least about or at most about: 30° C.-35° C., 33° C.-36° C., 34° C.-36° C., 34° C.-37° C., 34.5° C.-36.5° C., or 33.5° C.-36.5° C.

In some embodiments, the co-culturing microorganism is viable under conditions suitable for the growth of spirulina. For example, the co-culturing microorganism may be viable at about 9.8-10 pH, 9.9-10.5 pH, 10-11 pH, or 9.7-10.5 pH. In some embodiments, the co-culturing microorganism is viable at a pH of at least about or at most about: 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11.

In some embodiments, the co-culturing microorganism is viable at: light levels of between about 500 and about 2500 μmol/m2/sec, about 35° C., 9.8-10 pH, and combinations thereof. In some embodiments, the co-culturing microorganism is viable when cultured in cyanobacterial SOT media. In some embodiments, the co-culturing microorganism is viable when cultured in cyanobacterial SOT media in multitron conditions.

In some embodiments, the co-culturing microorganism comprises a bacterium of an order selected from the group consisting of: Micrococcales, Xanthomonadales, Purple sulfur bacteria, Nevskiales, Hyphomicrobiales, Mycobacteriales, Bacillales, Nitrosomonadales, Oceanospirillales, Oscillatoriales, and combinations thereof. In some embodiments, the co-culturing microorganism comprises a bacterium of an order selected from the group comprising: Micrococcales, Xanthomonadales, Purple sulfur bacteria, Nevskiales, Hyphomicrobiales, Mycobacteriales, Bacillales, Nitrosomonadales, Oceanospirillales, Oscillatoriales, or combinations thereof.

In some embodiments, the co-culturing microorganism belongs to the genus Sphingomonas. In some embodiments, the co-culturing microorganism is selected from: Sphingomonas abaci, S. adhaesiva, S. aerolata, S. aquatilis, S. asaccharolytica, S. aurantiaca, S. azotifigens, S. cloacae, S. dokdonensis, S. echinoides, S. elodea, S. faeni, S. genosp. 1, S. genosp. 2, S. koreensis, S. mali, S. melonis, S. mucosissima, S. oligophenolica, S. panni, S. parapaucimobilis, S. paucimobilis, S. phyllosphaerae, S. pituitosa, S. pruni, S. rhizogenes, S. roseiflava, S. sanguinis, S. suberifaciens, S. taejonensis, S. trueperi, S. ursincola, S. wittichii, S. xenophaga, S. yabuuchiae, S. yunnanensis, and Sphingomonas sp. In some embodiments, the co-culturing microorganism is selected from the group comprising: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus, Oscillatoria, Koinonema, Oxynema, Planktothrix, Microcystis, or combinations thereof.

In some embodiments, the co-culturing microorganism belongs to the genus Microcella. In some embodiments, the co-culturing microorganism is selected from M alkaliphile, and M putealis. In some embodiments, the co-culturing microorganism is of a genus selected from the group consisting of: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus and Oscillatoria, Koinonema, Oxynema, Planktothrix, Microcystis, and combinations thereof. In some embodiments, the co-culturing microorganism is of a genus selected from the group comprising: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus and Oscillatoria, Koinonema, Oxynema, Planktothrix, Microcystis, or combinations thereof. In some embodiments, a co-culturing microorganism belongs to the genus Microcella.

In some embodiments, a co-culturing microorganism comprises a small molecule mimetic of a functional fragment of a microorganism provided herein. In some embodiments, a co-culturing microorganism comprises a nucleic acid for example a DNA or RNA. In some embodiments, a co-culturing micro-organism comprises mRNA. In some embodiments, a co-culturing microorganism is a factor implicated in cellular stress. In some embodiments, a co-culturing microorganism promotes upregulation of a pilus pathway in the spirulina. In some embodiments, a co-culturing microorganism promotes opening of pores in the spirulina cell wall and/or membrane.

In some embodiments, a co-culturing microorganism is in direct contact with spirulina. In some embodiments, a co-culturing microorganism is not in direct contact with spirulina, for example under conditions where both the spirula and microorganism share a culture medium but do not contact each other.

Any number of co-culturing microorganisms may be utilized in compositions and methods of the disclosure. In some embodiments from about 1, 2, 3, 4, 5, 6, 7, 8, 9, or up to about 10 microorganisms are utilized. In some embodiments, 2 co-culturing microorganisms are utilized. Co-culturing microorganisms may belong to the same or different phylum, class, order, family, genus, or species. In some embodiments, a co-culturing microorganism belongs to the genus Microcella. In some embodiments, a co-culturing microorganism belongs to the genus sphingomonas.

In some embodiments, a composition comprises at least a portion of a co-culturing microorganism in an amount effective to induce competence of spirulina. One of skill in the art can measure for competence, in various ways, to develop the effective amount needed. Any amount of a co-culturing microorganism may be utilized. In some embodiments, about 10, 100, 1000, 1×10⁴, 1×10⁵, 1×10⁶, 1×10⁷, 1×10⁹, 1×10¹⁰, 1×10¹¹, 1×10¹², 1×10¹³, 1×10¹⁴, 1×10¹⁵, 10×10⁵, 10×10¹⁰, 10×10⁵⁰, 30×10¹⁰, 40×10¹⁰, 50×10¹⁰, or up to about 100×10¹⁰ per mL. 10, 100, 1000, 1×10⁴, 1×10⁵, 1×10⁶, 1×10⁷, 1×10⁹, 1×10¹⁰, 1×10¹¹, 1×10¹², 1×10¹³, 1×10¹⁴, 1×10¹⁵, 10×10⁵, 10×10¹⁰, 10×10⁵⁰, 30×10¹⁰, 40×10¹⁰, 50×10¹⁰, or up to about 100×10¹⁰ per L. In some embodiments, at least about or at most about 10, 100, 1000, 1×10⁴, 1×10⁵, 1×10⁶, 1×10⁷, 1×10⁹, 1×10¹⁰, 1×10¹¹, 1×10¹², 1×10¹³, 1×10¹⁴, 1×10¹⁵, 10×10⁵, 10×10¹⁰, 10×10⁵⁰, 30×10¹⁰, 40×10¹⁰, 50×10¹⁰, or up to about 100×10¹⁰ total co-culturing microorganisms are utilized in a composition.

In some embodiments, the identity of a co-culturing microorganism is identified via sequencing of the 16S and/or 23 S rRNA. In some embodiments, the species, genera and even phyla present in a composition may be unknown at the time of sequencing, and the goal of sequencing is to determine this microbial composition. In some embodiments, an analysis to determine identity of a co-culturing microorganism is selected from the group consisting of: BLAST, MEGAN, Naïve Bayes Classifier (NBC), Kraken, MetaPhlAn, and combinations thereof. In some embodiments, an analysis to determine identity of a co-culturing microorganism is selected from the group comprising: BLAST, MEGAN, Naïve Bayes Classifier (NBC), Kraken, MetaPhlAn, or combinations thereof. In some embodiments, a Kraken analysis is utilized to identify and/or select a co-culturing microorganism.

### Transforming Molecule

For natural transformation, the spirulina is grown in the presence of one or more co-culturing microorganisms and one or more transforming molecules. As used herein, the term “transforming molecule” describes any molecule that may be transformed or transfected into a host cell, including, but not limited to, polynucleotide sequences, double stranded polynucleotide sequences, single-stranded polynucleotide sequences, DNA, cDNA, plasmids, linearized vectors or plasmids, linear sequences of a polynucleotide, and barcoded polynucleotide sequences. In the methods described herein, the spirulina cell and/or spirulina cell population may be transformed with one or more different transforming molecules.

In some embodiments, the transforming molecule comprises one or more sequences for insertion into the spirulina genome. In some embodiments, the transforming molecule comprises one or more sequences for insertion into the spirulina genome that encodes a polypeptide or fragment thereof. In some embodiments, the polypeptide or fragment thereof is an antibody. In some embodiments, the antibody is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof. In some embodiments, an antibody or fragment thereof binds a coronavirus. In embodiments, an antibody, such as a VHH of the disclosure, targets any one of the four structural proteins of SARS-CoV-2 including but not limited to: spike protein (S), envelope protein (E), membrane protein (M) and/or nucleocapsid protein (INI). The spike protein (S protein) is responsible for receptor-recognition, attachment to the cell, infection via the endosomal pathway, and the genomic release driven by fusion of viral and endosomal membranes. Though sequences between the different family members vary, there are conserved regions and motifs within the S protein making it possible to divide the S protein into two subdomains: S1 and S2. While the S2, with its transmembrane domain, is responsible for membrane fusion, the S1 domain recognizes the virus-specific receptor and binds to the target host cell. In embodiments, the VHHs of the disclosure recognize the receptor binding domain (RBD) or spike protein of SARS-CoV-2. In some embodiments, a strain of SARS-CoV-2 is one or more of: alpha, beta, gamma, delta, epsilon, zeta, eta, theta, iota, kappa, lambda, mu, nu, xi, omicron, pi, rho, sigma, tau, upsilon, phi, chi, psi, or omega. In some embodiments, a pharmaceutical composition generated using methods of the disclosure is effective at reducing or eliminating COVID when a SARS-CoV-2 infection evades vaccine therapy.

Various antibodies and portions thereof that can be utilized in the present compositions and methods are described in WO2021003456A1 herein all of which is incorporated by reference.

In some embodiments, the polypeptide or fragment thereof is a native spirulina polypeptide. In some embodiments, the native spirulina polypeptide is a component of the phycobilisome. In some embodiments, the native spirulina polypeptide or fragment thereof is a phycocyanin or a phycoerythrin.

In some embodiments, the polypeptide or fragment thereof is a prophylactic or therapeutic polypeptide or a fragment thereof. In some embodiments, the polypeptide is an endogenous spirulina polypeptide. In some embodiments, the transforming molecule comprises multiple copies of sequence encoding an endogenous spirulina polypeptide. In some embodiments, the transforming molecule comprises multiple copies of sequence encoding an endogenous spirulina polypeptide in tandem. In some embodiments, the transforming molecule comprises multiple copies of an endogenous spirulina polypeptide separated by a linker or spacer.

In some embodiments, the transforming molecule comprises one or more sequences encoding an exogenous spirulina polypeptide. In some embodiments, the transforming molecule comprises multiple copies of sequence encoding an exogenous spirulina polypeptide. In some embodiments, the transforming molecule comprises multiple copies of sequence encoding an exogenous spirulina polypeptide in tandem. In some embodiments, the transforming molecule comprises multiple copies of an exogenous spirulina polypeptide separated by a linker or spacer.

In some embodiments, the exogenous polypeptide or fragment thereof is derived from a different bacteria or plant. In some embodiments, the exogenous polypeptide is selected from the group consisting of: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin. In some embodiments, the exogenous polypeptide is selected from the group comprising: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, or chlorotoxin. In some embodiments, the exogenous polypeptide is an antigen or epitope. In some embodiments, the antigen or epitope is derived from an infectious microorganism, a tumor antigen or a self-antigen associated with an autoimmune disease.

In some embodiments, the polypeptide or a fragment thereof is in a fusion protein. In some embodiments, the fusion protein comprises a carrier protein. In some embodiments, the carrier protein is selected from the group consisting of: maltose binding protein, hedgehog hepatitis virus-like particle, thioredoxin, and phycocyanin. thereof is in a fusion protein. In some embodiments, the fusion protein comprises a carrier protein. In some embodiments, the carrier protein is selected from the group comprising: maltose binding protein, hedgehog hepatitis virus-like particle, thioredoxin, or phycocyanin. In some embodiments, the fusion protein comprises a scaffold protein. In some embodiments, the polypeptide or fragment thereof is linked to a scaffold protein at the N-terminus or the C-terminus, or in the body of the scaffold protein. In some embodiments, the scaffold protein is selected from the oligomerization domain of C4b-binding protein (C4BP), cholera toxin b subunit, or oligomerization domains of extracellular matrix proteins.

In some embodiments, the prophylactic or therapeutic polypeptide and the scaffold protein are separated by about 1 to about 50 amino acids. In some embodiments, the fusion protein comprises multiple copies of the at least one polypeptide or fragment thereof, wherein the at least one exogenous polypeptide or fragment thereof and the scaffold protein are arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the at least one exogenous polypeptide or fragment thereof, SP is the scaffold protein, n, n1, and n2 represent the number of copies of the at least one exogenous polypeptide or fragment thereof.

In some embodiments, the polypeptide or fragment thereof is monomeric. In some embodiments, the polypeptide or fragment thereof is multimeric. In some embodiments, the polypeptide or fragment thereof is trimeric. In some embodiments, the polypeptide or fragment thereof is heteromeric. In some embodiments, the polypeptide or fragment thereof is homomeric. In some embodiments, the polypeptide or fragment thereof is arranged in a nanoparticle.

In some embodiments, the transforming molecule comprises one or more of a promoter, an enhancer, a transcription factor, a terminator, and a selectable marker. In some embodiments, the promoter is a strong promoter, an inducible promoter, a week promoter or a constitutive promoter. In some embodiments, a weak promoter is utilized in cases of toxicity associated with a transgene encoded by a transforming molecule.

In some embodiments, the vector or linear polynucleotide comprises one or more homology arms. In some embodiments, the vector or linear polynucleotide comprises one homology arm. In some embodiments, the vector or linear polynucleotide comprises two homology arms. In some embodiments, the single homology arm facilitates a single crossover event to insert the sequence adjacent to the homology arm into the spirulina genome. In some embodiments, the vector or linear polynucleotide comprises two homology arms that are adjacent to the sequence to be inserted into the spirulina genome. In some embodiments, the vector or linear polynucleotide comprises two homology arms that flank the sequence to be inserted into the spirulina genome. In some embodiments, the vector or linear polynucleotide comprises one or more homology arms that are flush with a sequence to be inserted into the spirulina genome. In some embodiments, the vector or linear polynucleotide comprises one or more homology arms that are at least about 1, 2, 3, 4, 5, 6, 7, 8, 9, 10, 20, 30, 40, or up to about 50 bases upstream and/or downstream of a sequence to be inserted into the spirulina genome. In some embodiments, the two homology arms that flank the sequence to be inserted into the spirulina genome facilitate a double crossover event for homologous recombination.

In some embodiments, the homology arms may be the same length. In some embodiments, the homology arms may be different lengths. In some embodiments, one or both of the homology arms may be at least about 50 bp to about 4000 bp. In some embodiments, one or both of the homology arms may be at least about 500 bp, at least about 1000 bp, at least about 1500 bp, or at least about 2000 bp. In some embodiments, the homology arm is about 500 bp, about 600 bp, about 700 bp, about 800 bp, about 900 bp, about 1000 bp, about 1100 bp, about 1200 bp, about 1300 bp, about 1400 bp, about 1500 bp, about 1600 bp, about 1700 bp, about 1800 bp, about 1900 bp, or about 2000 bp. In some embodiments, the homology arm is between 1000 bp and 1500 bp.

In some embodiments, the vector or linear polynucleotide sequence inserts a sequence into the spirulina genome. In some embodiments, the vector or linear polynucleotide sequence inserts a sequence into a targeted (e.g., predetermined) site in the spirulina genome. In some embodiments, the vector or linear polynucleotide sequence inserts a sequence into a targeted locus in the spirulina genome. In some embodiments, the vector or linear polynucleotide sequence inserts a sequence into a targeted coding region in the spirulina genome. In some embodiments, the vector or linear polynucleotide sequence inserts a sequence into a targeted non-coding region. In some embodiments, the vector or linear polynucleotide sequence inserts a sequence into a targeted upstream or promoter region. In some embodiments, the vector or linear polynucleotide sequence inserts a sequence into a targeted downstream or terminator region. In some embodiments, insertion of a sequence from a vector or polynucleotide sequence into the spirulina genome does not include insertion of vector backbone sequences into the spirulina genome. Any sequence of the spirulina genome may be targeted. In some embodiments, a sequence that is inconsequential for the growth and/or well-being of the spirulina is targeted, such as a safe-harbor gene.

In some embodiments, one or more of a clustered regularly interspaced short palindromic repeats (CRISPR), TALEN, transposon-based, ZEN, meganuclease, Mega-TAL molecules and/or transgenes are used to target a spirulina cell genome.

In some embodiments, a CRISPR system is utilized to target a spirulina genome. In some embodiments, a CRISPR system comprises an endonuclease and one or more guide nucleic acids. In some embodiments, a guide nucleic acid comprises a guide RNA. In some embodiments, a guide nucleic acid comprises a guide DNA. In some embodiments, a guide nucleic acid is a guide RNA (gRNA) that is capable of binding a protospacer adjacent motif (PAM) sequence in a spirulina genomic sequence. In some embodiments, a gRNA targets any of the spirulina sequences described herein. A guide RNA can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30 or more nucleotides. A target nucleic acid can be at most or at most about 5, 10, 15, 16, 17, 18, 19, 20, 21, 22, 23, 24, 25, 30 or more nucleotides. In some embodiments, a guide nucleic acid can comprise a nucleotide sequence (e.g., a spacer), for example, at or near the 5′ end or 3′ end, that can hybridize to a sequence in a target nucleic acid (e.g., a protospacer). A spacer of a guide nucleic acid can interact with a target nucleic acid in a sequence-specific manner via hybridization (i.e., base pairing). A spacer sequence can hybridize to a target nucleic acid that is located 5′ or 3′ of a protospacer adjacent motif (PAM). The length of a spacer sequence can be at least or at least about 5, 10, 15, 16, 17, 18, 19, 20, 21, 22, 23, 24, 25, 30 or more nucleotides. The length of a spacer sequence can be at most or at most about 5, 10, 15, 16, 17, 18, 19, 20, 21, 22, 23, 24, 25, 30 or more nucleotides. In some embodiments, a gRNA can be introduced at any functional concentration. For example, a gRNA can be introduced to a spirulina cell at 10 micrograms. In other cases, a gRNA can be introduced from 0.5 micrograms to 100 micrograms. A gRNA can be introduced from 0.5, 5, 10, 15, 20, 25, 30, 35, 40, 45, 50, 55, 60, 65, 70, 75, 80, 85, 90, 95, or 100 micrograms.

In some embodiments, a CRISPR system comprises an endonuclease. An endonuclease can be a Cas protein. A Cas protein can be selected from a list comprising Cas1, Cas1B, Cas2, Cas3, Cas4, Cas5, Cash, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof. In some cases, a CRISPR endonuclease can be Cas9. A Cas9 of the present disclosure can bind a protospacer adjacent motif (PAM) sequence. In some embodiments, a Cas is a dead Cas. In some embodiments, a guide RNA is used in conjunction with a catalytically dead Cas enzyme (dCas9) to repress expression of a host protein. This methodology may be useful to decrease expression in the host (e.g., spirulina) to increase growth rate, increase expression of a recombinant protein expression by decreasing expression of host proteins to allow more resources for recombinant proteins, or by attenuating the expression of proteases to prevent degradation of a recombinant protein. In some embodiments, an antibody or fragment thereof is introduced into a genome of spirulina adjacent to a PAM sequence of spirulina.

In some embodiments, the vector or linear polynucleotide sequence at least partially deletes a sequence from the spirulina genome. In some embodiments, the vector or linear polynucleotide sequence at least partially deletes a sequence from the spirulina genome. In some embodiments, the vector or linear polynucleotide sequence deletes a sequence from a targeted (e.g., predetermined) site in the spirulina genome. In some embodiments, the vector or linear polynucleotide sequence deletes a sequence from a targeted locus in the spirulina genome. In some embodiments, the vector or linear polynucleotide sequence deletes a sequence from a targeted coding region in the spirulina genome. In some embodiments, the vector or linear polynucleotide sequence deletes a sequence from a targeted non-coding region. In some embodiments, the vector or linear polynucleotide sequence deletes a sequence from a targeted upstream or promoter region. In some embodiments, the vector or linear polynucleotide sequence deletes a sequence from a targeted downstream or terminator region. In some embodiments, deletion of a sequence from the spirulina genome does not include insertion of vector backbone sequences into the spirulina genome.

In some embodiments, the spirulina and/or spirulina cell population is transformed with two or more different transforming molecules. In some embodiments, the spirulina and/or spirulina cell population is transformed with two or more different transforming molecules in the same round of transformation. In some embodiments, the spirulina and/or spirulina cell population is transformed with two or more different transforming molecules in the same round of transformation. In some embodiments, the spirulina and/or spirulina cell population is transformed with two or more different transforming molecules in multiple rounds of transformation. In some embodiments, a method comprises transforming one or more transforming molecules simultaneously and selecting for spirulina strains that have incorporated the one or more transforming molecules.

In some embodiments, the spirulina and/or spirulina cell population is transformed with at least 2 different transforming molecules in 2 rounds of transformation. In some embodiments, the first transformation inserts one transforming molecule into the spirulina genome and the second transformation replaces the first transforming molecule with a different transforming molecule inserted into the spirulina genome. In some embodiments, this method produces a markerless recombinant spirulina cell and/or spirulina cell population.

In some embodiments, a composition comprises at least about or at most about 50 ng, 150 ng, 200 ng, 250 ng, 300 ng, 350 ng, 400 ng, 450 ng, 500 ng, 550 ng, 600 ng, 650 ng, 700 ng, 750 ng, 800 ng, 850 ng, 900 ng, 950 ng, 1000 ng of a transforming molecule in a 30-404, reaction. This amount can be scaled up or down proportionally according to the desired scale of transformation. In some embodiments, a composition comprises about 300 ng of a transforming molecule per a 30-404, reaction.

### Transformation of Spirulina

In some embodiments, the present disclosure provides natural (e.g., non-chemical or non-electroporative) methods of transforming and modifying a spirulina cell. In some embodiments, the natural transformation is achieved by co-culturing the spirulina cell with one or more co-culturing microorganisms, and a transforming molecule. Also provided are non-natural (e.g., chemical or electroporative) methods of transforming and modifying a spirulina cell.

Any appropriate spirulina culture media may be used. In some embodiments, the culture media is SOT media. In some embodiments, the culture media includes, but is not limited to, SOT media, SAG media, BG-11 media, Georgia's media, Zarrouk's media, Hiri's media, and Jourdan's media or a combination thereof.

In some embodiments, the culture comprises a pH balancer or buffer. The pH balancer may be any suitable buffer that maintains viability of spirulina while keeping pH of the media between 6 and 10 pH, between 6.5 and 8.5 pH, or between 7 and 8 pH. Suitable pH balancers include, but are not limited to, HEPES, sodium or potassium phosphate buffer, and TES. In an embodiment the pH balancer may be HEPES-NaOH adjusted to a pH of 7.5.

In some embodiments, the culture comprising the spirulina, co-culture microorganism, and transforming molecule is grown with shaking. In some embodiments, the culture comprising the spirulina, co-culture microorganism, and transforming molecule is grown with shaking at about 20 rpm to about 500 rpm. In some embodiments, the culture comprising the spirulina, co-culture microorganism, and transforming molecule is grown with shaking at about 20 rpm, about 30 rpm, about 40 rpm, about 50 rpm, about 60 rpm, about 70 rpm, about 80 rpm, about 90 rpm, about 100 rpm, about 110 rpm, about 115 rpm, about 120 rpm, about 125 rpm, about 130 rpm, about 135 rpm, about 140 rpm, about 145 rpm, about 150 rpm, about 155 rpm, about 160 rpm, about 165 rpm, about 170 rpm, about 175 rpm, about 180 rpm, about 185 rpm, about 190 rpm, about 195 rpm, about 200 rpm, about 205 rpm, about 210 rpm, about 215 rpm, about 220 rpm, about 225 rpm, about 230 rpm, about 235 rpm, about 240 rpm, about 245 rpm, about 250 rpm, about 255 rpm, about 260 rpm, about 265 rpm, about 270 rpm, about 275 rpm, about 280 rpm, about 285 rpm, about 290 rpm, about 295 rpm, about 300 rpm, about 305 rpm, about 310 rpm, about 315 rpm, about 320 rpm, about 325 rpm, about 330 rpm, about 335 rpm, about 340 rpm, about 345 rpm, about 350 rpm, about 355 rpm, about 360 rpm, about 365 rpm, about 370 rpm, about 375 rpm, about 380 rpm, about 385 rpm, about 390 rpm, about 400 rpm, about 405 rpm, about 410 rpm, about 415 rpm, about 420 rpm, about 425 rpm, about 430 rpm, about 435 rpm, about 440 rpm, about 445 rpm, about 450 rpm, about 455 rpm, about 460 rpm, about 465 rpm, about 470 rpm, about 475 rpm, about 480 rpm, about 485 rpm, about 490 rpm, about 495, or about 500 rpm or more.

In some embodiments, the culture comprising the spirulina, co-culture microorganism, and transforming molecule is grown under illumination. In some embodiments, the culture comprising the spirulina, co-culture microorganism, and transforming molecule is grown under continuous illumination. In some embodiments, the culture comprising the spirulina, co-culture microorganism, and transforming molecule is grown under periodic or intermittent illumination. In some embodiments, the culture comprising the spirulina, co-culture microorganism, and transforming molecule is grown under illumination of about 10 μEi to about 3000 μEi. In some embodiments, the culture comprising the spirulina, co-culture microorganism, and transforming molecule is grown under illumination of about 50 μEi to about 110 μEi, or about 60 μEi to about 70 μEi, or about 110 μEi to about 150 μEi. In some embodiments, the culture comprising the spirulina, co-culture microorganism, and transforming molecule is grown under illumination of about 10 μEi, about 20 μEi, about 30 μEi, about 40 μEi, about 50 μEi, about 60 μEi, about 70 μEi, about 80 μEi, about 90 μEi, about 100 μEi, about 110 μEi, about 120 μEi, about 130 μEi, about 140 μEi, about 150 μEi, about 160 μEi, about 170 μEi, about 180 μEi, about 190 μEi, about 200 μEi, about 210 μEi, about 220 μEi, about 230 μEi, about 240 μEi, about 250 μEi, about 260 μEi, about 270 μEi, about 280 μEi, about 290 μEi, about 300 μEi, about 310 μEi, about 320 μEi, about 330 μEi, about 340 μEi, about 350 μEi, about 360 μEi, about 370 μEi, about 380 μEi, about 390 μEi, about 400 μEi, about 410 μEi, about 420 μEi, about 430 μEi, about 440 μEi, about 450 μEi, about 460 μEi, about 470 μEi, about 480 μEi, about 490 μEi, about 500 μEi, about 510 μEi, about 520 μEi, about 530 μEi, about 540 μEi, about 550 μEi, about 560 μEi, about 570 μEi, about 580 μEi, about 590 μEi, about 600 μEi, about 610 μEi, about 620 μEi, about 630 μEi, about 640 μEi, about 650 μEi, about 660 μEi, about 670 μEi, about 680 μEi, about 690 μEi, about 700 μEi, about 710 μEi, about 720 μEi, about 730 μEi, about 740 μEi, about 750 μEi, about 760 μEi, about 770 μEi, about 780 μEi, about 790 μEi, or about 800 μEi, about 810 μEi, about 820 μEi, about 830 μEi, about 840 μEi, about 850 μEi, about 860 μEi, about 870 μEi, about 880 μEi, about 890 μEi, about 900 μEi, about 910 μEi, about 920 μEi, about 930 μEi, about 940 μEi, about 950 μEi, about 960 μEi, about 970 μEi, about 980 μEi, about 990 μEi, or about 1000 μEi.

In some embodiments, the culture comprising the spirulina, co-culture microorganism, and transforming molecule is grown at a temperature of about 20° C. to about 40° C. In some embodiments, the culture comprising the spirulina, co-culture microorganism, and transforming molecule is grown at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In some embodiments, the culture comprising the spirulina, co-culture microorganism, and transforming molecule is grown at room temperature.

In some embodiments, the culture comprising the spirulina, co-culture microorganism, and transforming molecule is grown with about 0.01% to about 1.5% CO₂. In some embodiments, the culture comprising the spirulina, co-culture microorganism, and transforming molecule is grown with about 0.01%, about 0.02%, about 0.03%, about 0.04%, about 0.05%, about 0.06%, about 0.07%, about 0.08%, about 0.09%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 1.0%, about 1.1%, about 1.2%, about 1.3%, about 1.4%, about 1.5% CO₂. In some embodiments, the culture comprising the spirulina, co-culture microorganism, and transforming molecule is grown with atmospheric CO₂. In some embodiments, the culture comprising the spirulina, co-culture microorganism, and transforming molecule is grown in liquid culture.

In some embodiments, the culture comprising the spirulina, co-culture microorganism, and transforming molecule is grown at a pH of about 8.0 to about 11.0. In some embodiments, the culture comprising the spirulina, co-culture microorganism, and transforming molecule is grown at a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, or about 11.

In some embodiments, the culture comprising the spirulina, co-culture microorganism, and transforming molecule is grown for at least about 1 week to about 3 months. In some embodiments, the culture comprising the spirulina, co-culture microorganism, and transforming molecule is grown for at least about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, or about 3 months. In some embodiments, the culture comprising the spirulina, co-culture microorganism, and transforming molecule is grown for at least about 7 days to about 60 days. In some embodiments, the culture comprising the spirulina, co-culture microorganism, and transforming molecule is grown for about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, or about 60 days. In some embodiments, the culture comprising the spirulina, co-culture microorganism, and transforming molecule is grown in liquid culture.

In some embodiments, following transformation a greater amount of an RNA transcript is generated from spirulina utilizing a co-culturing methodology comprising a co-culturing microorganism as compared to an otherwise comparable method lacking the co-culturing microorganism. In some embodiments, following transformation a greater amount of soluble recombinant protein is generated from spirulina utilizing a co-culturing methodology comprising a co-culturing microorganism as compared to an otherwise comparable method lacking the co-culturing microorganism. In some embodiments, a greater amount of soluble protein is generated from spirulina utilizing a methodology lacking electroporation or another non-natural methodology as compared to an otherwise comparable method utilizing electroporation or engineered methodologies. In some embodiments, following transformation at least about 0.1% to about 100% of soluble protein is generated from spirulina utilizing a natural, co-culturing methodology provided herein. In some embodiments, following transformation at least about 0.5% to about 20% of soluble protein is generated from spirulina utilizing a co-culturing methodology provided herein. In some embodiments, from about: 0.1% to 0.3%, 0.1% to 3%, 0.1% to 5%, 0.5% to 3%, 0.5% to 10%, 1% to 3%, 1% to 5%, 3% to 10%, 5% to 15%, 10% to 20%, 3% to 50%, or 30% to 80% is generated. In some embodiments, from about: 0.1%, 0.3%, 0.5%, 0.8%, 1%, 3%, 4%, 5%, 10%, 15%, 20%, 25%, 30%, 35%, 40%, 45%, 50%, 55%, 60%, 65%, 70%, 75%, 80%, 85%, 90%, 95%, or up to about 100% of soluble protein is generated.

In some embodiments, a culture of spirulina is healthier following transformation when the culture comprises a co-culturing microorganism as compared to an otherwise comparable method lacking the co-culturing microorganism. Health of spirulina can be determined using any means. In some embodiments, a culture of spirulina, pre- or post-transformation, has reduced cell clumping as compared to an otherwise comparable culture lacking a co-culturing microorganism. In some embodiments, a culture of spirulina, pre- or post-transformation, has increased growth as compared to an otherwise comparable culture lacking a co-culturing microorganism.

To facilitate delivery to the airway, or other targets, an antibody of the disclosure (e.g., a VHH) may be extracted from the spirulina by lysing the spirulina, partially purifying the soluble proteins including the VHH, and drying the partially purified protein by an appropriate drying method including any drying method provided herein. For example, the harvested spirulina slurry may be lysed using high pressure homogenization, and the soluble protein separated from insoluble materials by filtration, then diafiltered and concentrated by tangential flow ultrafiltration. The partially purified extract may be formulated with appropriate stabilizers and mucoadhesives, including carbohydrates such as trehalose, hyaluronic acid, or chitosan; amino acids such as lysine or glycine; and polymers such as polyvinyl alcohol or polyvinyl pyrrolidone. In embodiments, the VHH will comprise a purity from about: 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, or up to about 70% of solids.

### Culturing Conditions—Industrial Scale; Large Scale

In some embodiments, the culture comprising the spirulina, co-culture microorganism, and transforming molecule is grown in culture volumes of about 10 μL to about 2500 L. In some embodiments, the culture comprising the spirulina, co-culture microorganism, and transforming molecule is grown in culture volumes of about 10 μL, 100 μL, about 200 μL, about 300 μL, about 400 μL, about 500 μL, about 600 μL, about 700 μL, about 800 μL, about 900 μL, about 1000 μL, about 1 mL, about 10 mL, about 20 mL, about 30 mL, about 40 mL, about 50 mL, about 60 mL, about 70 mL, about 80 mL, about 90 mL, about 100 mL, about 110 mL, about 120 mL, about 130 mL, about 140 mL, about 150 mL, about 160 mL, about 170 mL, about 180 mL, about 190 mL, about 200 mL, about 300 mL, about 400 mL, about 500 mL, about 600 mL, about 700 mL, about 800 mL, about 900 mL, about 1000 mL, about 1 L, about 2 L, about 3 L, about 4 L, about 5 L, about 6 L, about 7 L, about 8 L, about 9 L, about 10 L, about 15 L, about 20 L, about 25 L, about 30 L, about 40 L, about 45 L, about 50 L, about 55 L, about 60 L, about 65 L, about 70 L, about L, about 80 L, about 85 L, about 90 L, about 95 L, about 100 L, about 110 L, about 120 L, about 130 L, about 140 L, about 150 L, about 160 L, about 170 L, about 180 L, about 190 L, about 200 L, about 210 L, about 220 L, about 230 L, about 240 L, about 250 L, about 260 L, about 270 L, about 280 L, about 290 L, about 300 L, about 310 L, about 320 L, about 330 L, about 340 L, about 350 L, about 360 L, about 370 L, about 380 L, about 390 L, about 400 L, about 410 L, about 420 L, about 430 L, about 440 L, about 450 L, about 460 L, about 470 L, about 480 L, about 490 L, about 500 L, about 600 L, about 700 L, about 800 L, about 900 L, or about 1000 L, or more.

In some embodiments, the spirulina are grown in large-scale continuous growth. Open pond systems are typically used to cultivate spirulina at commercial scale for production of food, feed, and pigments, but uncontrolled exposure to environmental contaminants make these challenging for the manufacture of biopharmaceuticals under FDA cGMP. However, the developments of an indoor, pH controlled, air-mixed photobioreactor platform based on a modular 160 L-2,000 L vertical flat panel reactor that is scalable to commercial size suitable for the manufacturing of biopharmaceuticals may solve this problem. The major advantage of this type of platform includes exceptionally low cost of large-scale growth and downstream processing. Additionally, spirulina may be grown in unsealed reactors under sanitary, but not aseptic, conditions.

In some embodiments, cultures of spirulina are continuously maintained for at least 1 week. In some embodiments, cultures of spirulina are continuously maintained for sequential growth cycles. In some embodiments, cultures of spirulina are continuously maintained for sequential one-week, two-week, three-week, four-week, five-week, six-week, seven-week, eight-week, or longer growth cycles.

### Pharmaceutical Compositions and Dosing

The spirulina disclosed herein may be used in a pharmaceutical composition for administration to a subject in need thereof. In some embodiments, the pharmaceutical composition is administered by any appropriate route. In some embodiments, the pharmaceutical composition is administered orally, parenterally, nasally, or via inhalation.

As used herein, the terms “oral composition” or “orally delivered composition” comprise compositions administered or delivered to the gastrointestinal tract (e.g. orally, compositions administered to the stomach via a feeding tube, etc.). Any appropriate area of the gastrointestinal tract may be targeted by the compositions of the present disclosure.

In some embodiments, the compositions of the present disclosure are administered topically. In some embodiments, the compositions of the present disclosure are administered via the airway. In some embodiments, the compositions of the present disclosure are administered by inhalation. In some embodiments, the compositions of the present disclosure are administered intranasally. In some embodiments, the compositions of the present disclosure are administered by a nebulizer, an inhaler, or a mist. In some embodiments, the compositions of the present disclosure are lyophilized and delivered as a powder or a powder resuspended in a liquid.

In some embodiments, the compositions of the present disclosure are formulated for topical administration. In some embodiments, the compositions of the present disclosure are formulated for administration via the airway. In some embodiments, the compositions of the present disclosure are formulated for administration by inhalation. In some embodiments, the compositions of the present disclosure are formulated for intranasal administration. In some embodiments, the compositions of the present disclosure are formulated for administration by a nebulizer, an inhaler, or a mist.

In some embodiments, compositions of the present disclosure can comprise one or more pharmaceutically acceptable excipients. Pharmaceutically acceptable carriers include but are not limited to saline, buffered saline, dextrose, water, glycerol, sterile isotonic aqueous buffer, and combinations thereof. In some embodiments, a pharmaceutically acceptable excipient is sodium bicarbonate.

In some embodiments, compositions of the present disclosure may comprise an adjuvant. As known in the art, the immunogenicity of a particular composition can be enhanced by the use of non-specific stimulators of the immune response, known as adjuvants. Exemplary adjuvants include a water-in-oil (W/O) emulsion composed of a mineral oil and a surfactant from the mannide monooleate family (e.g., MONTANIDE™ class of adjuvants) and flagellin adjuvants.

In some embodiments, compositions of the present disclosure comprise about 0.1% to about 5% of the total spirulina biomass. In some embodiments, compositions of the present disclosure comprise about 1 mg to about 50 mg of the exogenous antigenic epitope per gram of dried spirulina biomass. In some embodiments, compositions of the present disclosure comprise at least about 1 mg, 5 mg, 10 mg, 25 mg, 50 mg, 100 mg, 200 mg, 300 mg, 500 mg, 750 mg, 1 mg, 5 mg, 10 mg, or 50 of the exogenous antigenic epitope per gram of dried spirulina biomass.

### Uses of Compositions

In some embodiments, compositions of the present disclosure can be used to reduce the severity of a disease or disorder in a subject in need thereof. In some embodiments, compositions can be used to prevent a disease or disorder in a subject. In some embodiments, compositions can be used to prevent initiation of a disease or disorder in a subject. In some embodiments, compositions can be used to reduce the severity of a disease or disorder in a subject. In some embodiments, compositions can be used to prevent or delay recurrence of a disease in a subject. In some embodiments, compositions can be used to treat, prevent, or delay recurrence of a cancer in a subject.

In some embodiments, compositions and methods of the disclosure can be used to treat or prevent an infection. An infection can be of any microorganism including but not limited to: bacterial, fungal, algal, protozoal, and viral. In some embodiments, the infection is bacterial. In some embodiments, the infection, disease, or disorder is of a mucosal tract including but not limited to: mouth, nose, eyelids, trachea (windpipe) and lungs, stomach and intestines, and the ureters, urethra, vaginal, and urinary bladder. In some embodiments, the disease or disorder is of the gastrointestinal tract.

Compositions of the present disclosure can be used as a vaccine. In some embodiments, compositions can be used to induce an immune response in a subject. For example, compositions can be used to induce an immune response directed to an infectious microorganism, a tumor antigen, or a self-antigen.

The compositions of the present disclosure may be administered daily, weekly, biweekly, every other week, monthly, etc. In some embodiments, the compositions of the present disclosure are administered to a subject for about 1 day to about 1 year. In some embodiments, the compositions of the present disclosure are administered to a subject for about 1 day, 2 days, 3 days, 4 days, 5 days, 6 days, 7 days, 8 days, 9 days, 10 days, one week, two weeks, three weeks, four weeks, five weeks, six weeks, one month, two months, three months, four months, five months or more. In some embodiments, the compositions of the present disclosure are administered on consecutive days. In some embodiments, the compositions of the present disclosure are administered on non-consecutive days. In some embodiments, the compositions of the present disclosure are administered once a day. In some embodiments, the compositions of the present disclosure are administered multiple times a day. In some embodiments, the compositions of the present disclosure are administered twice a day, three times a day, four times a day, or more. In some embodiments, the compositions of the present disclosure are administered continuously (e.g., via a feeding tube). In some embodiments, the compositions of the present disclosure are administered with meals. In some embodiments, the compositions of the present disclosure are administered when the subject is in a fasting state

### Containers

Provided herein are also containers comprising any of the compositions provided herein. In some embodiments, a container comprises a bioreactor. In some embodiments, a container comprises a kit. In some embodiments, a container comprises a vial.

In some embodiments, a container comprises a kit. Kits can include packaging, instructions, and any of the compositions provided herein. In some embodiments, kits can also contain additional compositions used to generate the compositions described. In some embodiments, a kit comprises one or more of: (a) spirulina; (b) at least a portion of a co-culturing microorganism; (c) a growth or storage medium; and/or (d) instructions for using the same.

### Definitions

By “about” is meant a quantity, level, value, number, frequency, percentage, dimension, size, amount, weight, or length that varies by as much as 30, 25, 20, 15, 10, 9, 8, 7, 6, 5, 4, 3, 2, or 1% to a reference quantity, level, value, number, frequency, percentage, dimension, size, amount, weight, or length.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds) and provided literal support for and includes the end points of 10 and 100.

The present description uses specific numerical values to quantify certain parameters relating to the invention, where the specific numerical values are not expressly part of a numerical range. It should be understood that each specific numerical value provided herein is to be construed as providing literal support for a broad, intermediate, and narrow range. The broad range associated with each specific numerical value is the numerical value plus and minus 60 percent of the numerical value, rounded to two significant digits. The intermediate range associated with each specific numerical value is the numerical value plus and minus 30 percent of the numerical value, rounded to two significant digits. The narrow range associated with each specific numerical value is the numerical value plus and minus 15 percent of the numerical value, rounded to two significant digits. These broad, intermediate, and narrow numerical ranges should be applied not only to the specific values, but should also be applied to differences between these specific values.

The recitation “polynucleotide” or “nucleic acid” as used herein designates mRNA, RNA, cRNA, rRNA, cDNA, or DNA. The term typically refers to polymeric form of nucleotides of at least 10 bases in length, either ribonucleotides or deoxynucleotides or a modified form of either type of nucleotide. The term includes single and double stranded forms of DNA and RNA.

As used herein, the term “DNA” includes a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

With regard to polynucleotides, the term “exogenous” refers to a polynucleotide sequence that does not naturally occur in a wild-type cell or organism but is typically introduced into the cell by molecular biological techniques. Examples of exogenous polynucleotides include vectors, plasmids, and/or man-made nucleic acid constructs encoding a desired protein. With regard to polynucleotides, the term “endogenous” or “native” refers to naturally occurring polynucleotide sequences that may be found in a given wild-type cell or organism. A vector, plasmid, or other man-made construct that includes an endogenous polynucleotide sequence combined with polynucleotide sequences of the unmodified vector etc. is, as a whole, an exogenous polynucleotide and may also be referred to as an exogenous polynucleotide including an endogenous polynucleotide sequence. Also, a particular polynucleotide sequence that is isolated from a first organism and transferred to second organism by molecular biological techniques is typically considered an “exogenous” polynucleotide with respect to the second organism.

Polynucleotides may comprise a native sequence (e.g., an endogenous sequence that encodes protein described herein) or may comprise a variant or fragment, or a biological functional equivalent of such a sequence. Polynucleotide variants may contain one or more substitutions, additions, deletions and/or insertions, as further described herein, preferably such that the enzymatic activity of the encoded polypeptide is not substantially diminished relative to the unmodified or reference polypeptide. The effect on the enzymatic activity of the encoded polypeptide may generally be assessed as described herein and known in the art.

As will be understood by those skilled in the art, the polynucleotide sequences of this disclosure can include genomic sequences, extra-genomic, and plasmid-encoded sequences and smaller engineered gene segments that express, or may be adapted to express, proteins, polypeptides, peptides and the like. Such segments may be naturally isolated, or modified synthetically by the hand of man.

Polynucleotides may be single-stranded (coding or antisense) or double-stranded, and may be DNA (genomic, cDNA, or synthetic) or RNA molecules. Additional coding or non-coding sequences may, but need not, be present within a polynucleotide of the present invention, and a polynucleotide may, but need not, be linked to other molecules and/or support materials.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The percent identity of two sequences, whether nucleic acid or amino acid sequences, is the number of exact matches between two aligned sequences divided by the length of the shorter sequences and multiplied by 100. An approximate alignment for nucleic acid sequences is provided by the Smith-Waterman algorithm. The Smith-Waterman algorithm can be applied to amino acid sequences by using a known scoring matrix (e.g., the scoring matrix developed by Dayhoff) and normalized by any well-known technique such as the Gribskov method. One implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Other suitable programs for calculating the percent identity or similarity between sequences are generally known in the art, for example, the MPSRCH package of programs copyrighted by the University of Edinburgh, developed by John F. Collins and Shane S. Sturrok, and distributed by IntelliGenetics, Inc. (Mountain View, Calif), and BLAST, used with default parameters. Details of these programs can be found at the following internet address: http://blast.ncbi.nlm.nih.gov/Blast.cgi

“Polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain embodiments, polypeptides may include enzymatic polypeptides, or “enzymes,” which typically catalyze (i.e., increase the rate of) various chemical reactions. Exemplary nucleotide sequences that encode the proteins and enzymes of the application encompass full-length reference polynucleotides, as well as portions of the full-length or substantially full-length nucleotide sequences of these genes or their transcripts or DNA copies of these transcripts. Portions of a nucleotide sequence may encode polypeptide portions or segments that retain the biological activity of the reference polypeptide.

“Transformation” refers to the stable, heritable alteration in a cell resulting from the uptake and incorporation of exogenous nucleotides into the host-cell genome; also, the transfer of an exogenous gene from one organism into the genome of another organism. Exogenous nucleotides may include gene foreign to the target organism or addition of a nucleotide sequence present in the wild-type organism.

“Targeted mutation” means a change in the DNA sequence of the genome at a pre-determined (specified) genome location. In some cases, a targeted mutation will involve the introduction of a pre-determined (specified) DNA sequence alteration at the pre-determined genome location. In other cases, a targeted mutation will involve the introduction of a random DNA sequence alteration at the pre-determined genome location.

“Stable” when describing the results of a genetic modification caused by transformation refers to a genetic modification that is maintained in at least a portion of a population of cells for ten or more generations or for a length of time equal or greater to ten times the average generation time for the modified organism.

### Examples of Non-Limiting Embodiments of the Disclosure

Embodiments of the present subject matter disclosed herein may be beneficial alone or in combination with one or more other embodiments. Without limiting the foregoing description, certain non-limiting embodiments of the disclosure are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

### Exemplary Embodiments:

Embodiment 1. A method of transforming a population of spirulina cells comprising growing the spirulina cells with: (a) a co-culturing microorganism to induce competence; and (b) a transforming molecule.
Embodiment 2. The method of embodiment 1, wherein the co-culturing microorganism is gram-negative.
Embodiment 3. The method of embodiment 1, wherein the co-culturing microorganism is gram-positive.
Embodiment 4. The method of any of embodiments 1-3, wherein the co-culturing microorganism is aerobic.
Embodiment 5. The method of any of embodiments 1-4, wherein the co-culturing microorganism belongs to the genus Sphingomonas.
Embodiment 6. The method of embodiment 5, wherein the co-culturing microorganism is selected from: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus and Oscillatoria, Koinonema, Oxynema, Planktothrix, Microcystis, and combinations thereof.
Embodiment 7. The method of any of embodiments 1-5, wherein the co-culturing microorganism belongs to the genus Microcella.
Embodiment 8. The method of embodiment 7, wherein the co-culturing microorganism is selected from M alkahphile, and M putealis.
Embodiment 9. The method of any one of embodiments 1-8, wherein transformation of the spirulina with the transforming molecule deletes one or more genes, loci, or sequences in the spirulina genome.
Embodiment 10. The method of any one of embodiments 1-9, wherein transformation of the spirulina with the transforming molecule adds one or more genes, loci, or sequences to the spirulina genome.
Embodiment 11. The method of any one of embodiments 1-9, wherein transformation of the spirulina with the transforming molecule replaces one or more genes, loci, or sequences in the spirulina genome with the transforming molecule.
Embodiment 12. The method of any one of embodiments 1-11, wherein the spirulina cell is transformed with multiple transforming molecules.
Embodiment 13. The method of any one of embodiments 1-12, wherein the spirulina is transformed with different transforming molecules in multiple rounds of transformation.
Embodiment 14. The method of embodiment 13, wherein the spirulina is transformed with at least 2 different transforming molecules in 2 rounds of transformation.
Embodiment 15. The method of embodiment 14, wherein the first transformation inserts one transforming molecule into the spirulina genome and the second transformation replaces the first transforming molecule with a different transforming molecule inserted into the spirulina genome.
Embodiment 16. The method of any of embodiments 1-15, wherein the transforming molecule is a polynucleotide.
Embodiment 17. The method of embodiment 16, wherein the polynucleotide is DNA.
Embodiment 18. The method of embodiment 17, wherein the DNA is cDNA.
Embodiment 19. The method of any of embodiments 16-18, wherein the polynucleotide is comprised in a vector.
Embodiment 20. The method of embodiment 19, wherein the vector is a circular vector.
Embodiment 21. The method of embodiment 19, wherein the vector is linearized.
Embodiment 22. The method of any of embodiments 16-18, wherein the polynucleotide is a liner polynucleotide.
Embodiment 23. The method of any of embodiments 1-22, wherein the transforming molecule contains one or more homology arms.
Embodiment 24. The method of embodiment 23, wherein the one or more homology arms flank a sequence to be inserted into the spirulina genome.
Embodiment 25. The method of embodiment 23 or 24, wherein the homology arm is between about 1000 and about 1500 nucleotides long.
Embodiment 26. The method of any of embodiments 16-25, wherein the polynucleotide comprises one or more promoters, terminators, or enhancer sequences.
Embodiment 27. The method of embodiment 26, wherein the promoter is selected from an inducible promoter, a constitutive promoter, and a strong promoter.
Embodiment 28. The method of any one of embodiments 1-27, wherein the recombinant spirulina express one or more polypeptides or fragments thereof.
Embodiment 29. The method of embodiment 28, wherein the polypeptide is an antibody or fragment thereof.
Embodiment 30. The method of embodiment 29, wherein the antibody or fragment thereof is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof.
Embodiment 31. The method of embodiment 30, wherein the antibody is a VHH antibody.
Embodiment 32. The method of embodiment 28, wherein the polypeptide or fragment thereof is a therapeutic or prophylactic polypeptide.
Embodiment 33. The method of embodiment 32, wherein the therapeutic or prophylactic polypeptide is intended for delivery to the gastrointestinal tract of a subject.
Embodiment 34. The method of embodiment 32, wherein the therapeutic or prophylactic molecule is intended for systemic delivery in a subject.
Embodiment 35. The method of any one of embodiments 32-34, wherein the therapeutic or prophylactic polypeptide is an endogenous spirulina polypeptide.
Embodiment 36. The method of embodiment 35, wherein the endogenous spirulina polypeptide is found in higher concentrations than found in naturally-occurring spirulina.
Embodiment 37. The method of any of embodiments 32-34, wherein the therapeutic or prophylactic polypeptide is exogenous to spirulina.
Embodiment 38. The method of embodiment 37, wherein the exogenous polypeptide is naturally produced by a different bacteria or plant.
Embodiment 39. The method of embodiment 37 or 38, wherein the exogenous polypeptide is selected from the group consisting of: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin.
Embodiment 40. The method of embodiment 37 or 38, wherein the exogenous polypeptide is an antigen or epitope.
Embodiment 41. The method of embodiment 40, wherein the antigen or epitope is derived from an infectious microorganism, a tumor antigen or a self-antigen associated with an autoimmune disease
Embodiment 42. The method of any one of embodiments 37-41, wherein the exogenous polypeptide or a fragment thereof is in a fusion protein.
Embodiment 43. The method of any one of embodiments 1-42, wherein the spirulina is transformed with a nucleic acid, and wherein at least 2, at least 3, at least 4, or at least 5 copies of a nucleic acid sequence encoding the at least one exogenous polypeptide or fragment thereof are present in the recombinant spirulina.
Embodiment 44. The method of any one of embodiments 1-43, wherein the spirulina is transformed with a nucleic acid, and wherein 2, 3, 4, 5, 6, 8, 10, 15, 20, 25, 30, 40, or 50 copies of a nucleic acid sequence encoding the at least one exogenous polypeptide or fragment thereof are present in the recombinant spirulina.
Embodiment 45. The method of embodiment 44, wherein the recombinant spirulina comprises at least 2, at least 3, at least 4, or at least 5 different exogenous polypeptides or fragments thereof.
Embodiment 46. The method of any of embodiments 42-45, wherein the fusion protein comprises a carrier protein.
Embodiment 47. The method of embodiment 46, wherein the carrier protein is selected from the group consisting of: maltose binding protein, hedgehog hepatitis virus-like particle, thioredoxin, and phycocyanin.
Embodiment 48. The method of embodiment 47, wherein the fusion protein comprises a scaffold protein.
Embodiment 49. The method of embodiment 48, wherein the at least one exogenous polypeptide is linked to a scaffold protein at the N-terminus or the C-terminus, or in the body of the scaffold protein.
Embodiment 50. The method of embodiment 48 or 49, wherein the scaffold protein is selected from the oligomerization domain of C4b-binding protein (C4BP), cholera toxin b subunit, or oligomerization domains of extracellular matrix proteins.
Embodiment 51. The method of any of embodiments 48 to 50, wherein the at least one exogenous polypeptide and the scaffold protein are separated by about 1 to about 50 amino acids.
Embodiment 52. The method of any of embodiments to 42-51, wherein the fusion protein comprises multiple copies of the at least one exogenous polypeptide or fragment thereof, wherein the at least one exogenous polypeptide or fragment thereof and the scaffold protein are arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the at least one exogenous polypeptide or fragment thereof, SP is the scaffold protein, n, n1, and n2 represent the number of copies of the at least one exogenous polypeptide or fragment thereof.
Embodiment 53. The method of any of embodiments 1-52, wherein the therapeutic or prophylactic molecule is monomeric.
Embodiment 54. The method of any one of embodiments 1-52, wherein the therapeutic or prophylactic molecule is multimeric.
Embodiment 55. The method of any one of embodiments 1-52, wherein the therapeutic or prophylactic molecule is trimeric.
Embodiment 56. The method of any one of embodiments 1-55, wherein the multimer is heteromeric.
Embodiment 57. The method of any one of embodiments 1-55, wherein the multimer is homomeric.
Embodiment 58. The method of any one of embodiments 1-57, wherein the multimer is arranged in a nanoparticle.
Embodiment 59. The method of any one of embodiments 1-58, wherein the spirulina is selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f acutissima, A. neapolitana, A. nordstedtii, A. oceanica, A. okensis, A. pellucida, A. platensis, A. platensis var. non-constricta, A. platensis f granulate, A. platensis f minor, A. platensis var. tenuis, A. santannae, A. setchellii, A. skujae, A. spirulinoides f tenuis, A. spirulinoides, A. subsalsa, A. subtilissima, A. tenuis, A. tenuissima, and A. versicolor.
Embodiment 60. The method of any one of embodiments 1-59, wherein transformation is achieved by growing the spirulina, co-culturing microorganism, and transforming molecule in liquid culture for between 1 and 4 weeks.
Embodiment 61. The method of embodiment 60, wherein the co-culture is grown in liquid culture for at least 2 weeks.
Embodiment 62. The method of embodiment 61, wherein the co-culture is grown in liquid culture for at least 3 weeks.
Embodiment 63. A culture of a population of recombinant spirulina cells created by the method of any one of embodiments 1-62.
Embodiment 64. A recombinant spirulina cell created by the method of any one of embodiments 1-62.
Embodiment 65. A method of transforming a population of spirulina cells comprising growing the spirulina cells (a) under conditions that induce competence, and (b) with a transforming molecule.
Embodiment 66. A composition that comprises: a. a population of spirulina cells;
b. at least a portion of a co-culturing microorganism in an amount effective to induce competence; and c. a transforming molecule.
Embodiment 67. The composition of embodiment 66, wherein the transforming molecule comprises a polynucleotide.
Embodiment 68. The composition of embodiment 67, wherein the polynucleotide comprises DNA.
Embodiment 69. The composition of embodiment 68, wherein the DNA is cDNA.
Embodiment 70. The composition of embodiment 69, wherein the cDNA comprises at least two sequences encoding a first and a second homology arm, and wherein the first and the second homology arm are between about 1000 and about 1500 nucleotides long.
Embodiment 71. The composition of embodiment 70, wherein the first and the second homology arm bind to a Spirulina sequence comprising at least a portion of a GNAT family N-acetyltransferase sequence.
Embodiment 72. The composition of embodiment 66, wherein the at least a portion of the co-culturing microorganism comprises the entire microorganism.
Embodiment 73. The composition of embodiment 66, wherein the at least a portion of the co-culturing microorganism comprises a portion of a microorganism.
Embodiment 74. The composition of embodiment 66, wherein at least about 5% of the spirulina cells in the population are transformed as determined by sequencing.
Embodiment 75. The composition of embodiment 70, wherein the first and the second homology arms flank a sequence encoding an antibody or fragment thereof
Embodiment 76. The composition of embodiment 75, wherein the antibody or fragment thereof is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof
Embodiment 77. The composition of embodiment 76, comprising the VHH antibody.
Embodiment 78. The composition of embodiment 77, wherein the VHH antibody binds a target in a gastrointestinal tract.
Embodiment 79. The composition of embodiment 78, wherein the target comprises a pathogen or cancer cell.
Embodiment 80. The composition of embodiment 79, comprising the pathogen, wherein the pathogen is a bacterium.
Embodiment 81. The composition of embodiment 80, wherein the bacterium comprises campylobacter.
Embodiment 82. The composition of embodiment 66, wherein the co-culturing microorganism is a bacterium.
Embodiment 83. The composition of embodiment 82, wherein the bacteria are gram positive.
Embodiment 84. The composition of embodiment 82, wherein the bacteria are gram negative.
Embodiment 85. The composition of embodiment 82, wherein the bacteria are of an order selected from the group consisting of: Micrococcales, Xanthomonadales, Purple sulfur bacteria, Nevskiales, Hyphomicrobiales, Mycobacteriales, Bacillales, Nitrosomonadales, Oceanospirillales, Oscillatoriales, and combinations thereof.
Embodiment 86. The composition of embodiment 82, wherein the bacteria are of a genus selected from the group consisting of: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus and Oscillatoria, Koinonema, Oxynema, Planktothrix, Microcystis, and combinations thereof.
Embodiment 87. The composition of embodiment 86, comprising Sphingomonas or Microcella.
Embodiment 88. The composition of embodiment 66, wherein when the composition comprises a volume from about 30 to about 40 μL, the composition comprises: a. about 0.1 to 1 OD of the spirulina cells when measured at 750 nm wavelength as determined by spectrophotometry; and b. about 275 ng to 325 ng of the transforming molecule.
Embodiment 89. A pharmaceutical generated using the method of Embodiment 1.
Embodiment 90. The pharmaceutical of embodiment 89, wherein the pharmaceutical is in unit dose form.
Embodiment 91. A method of treating a disease or disorder in a subject in need thereof, comprising administering the pharmaceutical of embodiment 85, thereby treating the disease or disorder.
Embodiment 92. The method of embodiment 91, wherein the disease or disorder is of a tract comprising a mucosal membrane.
Embodiment 93. The method of embodiment 92, wherein the tract comprises the gastrointestinal tract.
Embodiment 94. The method of embodiment 92, wherein the disease or disorder comprises an infection of Campylobacter jejuni.
Embodiment 95. The method of embodiment 91, wherein the administering is an oral administration.
Embodiment 96. A container that comprises the composition of embodiment 66 or embodiment 89.
Embodiment 97. The container of embodiment 96, wherein the container comprises a bioreactor.
Embodiment 98. A kit that comprises: (a) a composition that comprises spirulina; (b) at least a portion of a co-culturing microorganism; (c) a growth or storage medium; and (d) instructions for use thereof.

### EXAMPLES

### Example 1—Spirulina are Naturally Competent for Transformation

Spirulina are naturally competent for transformation despite being widely viewed as refractory to genetic manipulation.

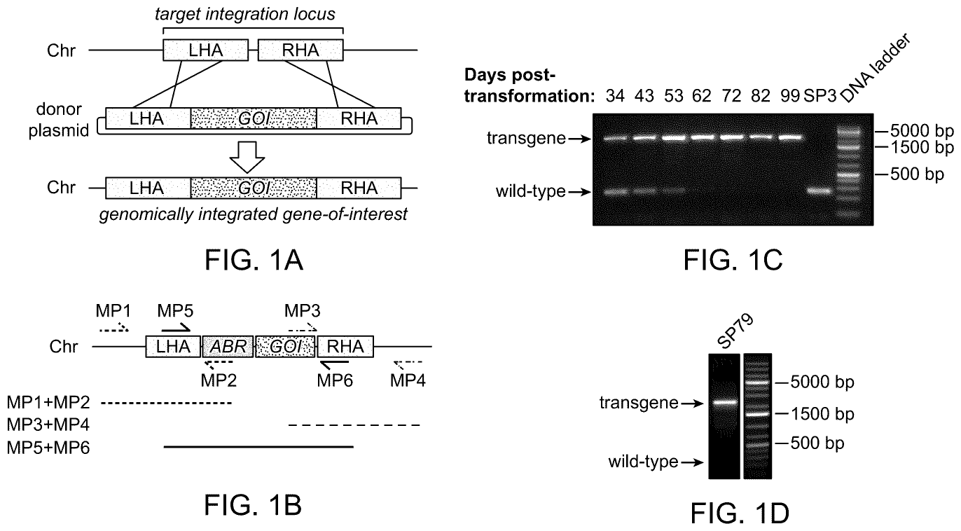

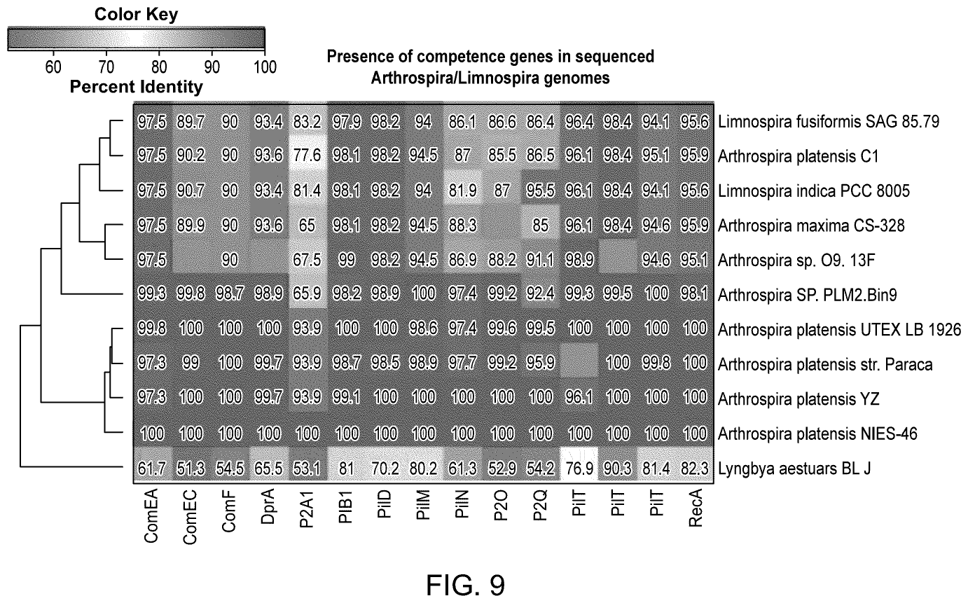

Efficient transformation was achieved by co-cultivation of spirulina with co-culturing microorganisms that induced competence (see below). Competent spirulina (UTEX LB1926 and NIES-39) were exposed to an integrating circular DNA vector containing an antibiotic resistance marker and a gene of interest flanked on both sides by sequences homologous to the spirulina chromosome. Spirulina were then maintained in liquid culture under antibiotic selection. Microscopic clusters of green cells were detected after two weeks of cultivation, and after three weeks fully green cultures were apparent. Precise integration of the transforming DNA into the spirulina genome by homologous recombination was demonstrated by PCR amplification and DNA sequencing of chromosomal DNA using primers flanking the insertion site (FIG. 1A and FIG. 1B). This natural transformation method yielded a pool of approximately 100 independent transformants, as determined by next-generation sequencing of a culture that had been exposed to a library of bar-coded, but otherwise identical, integrating DNA vectors. Consistent with these observations, the 15 genes associated with the pathway for natural competence in cyanobacteria are present as complete open reading frames in the genomes of all available Arthrospira genomes, including UTEX LB1926 and NIES-39, see FIG. 9.

Spirulina are polyploid. Segregation of the transgene to homozygosity typically occurred at about 6 weeks after transformation under continuous antibiotic selection (FIG. 1C). Clonal derivatives were then isolated by picking individual spirulina filaments under a microscope and verified as containing a single precisely integrated insertion per spirulina chromosome (see Methods). Long-term stability was assessed for 9 strains continuously propagated for at least 1 year (>800 cell generations). PCR and DNA sequencing showed all strains to be genetically indistinguishable from the original engineered strain. One strain, expressing an exogenous vaccine antigen, has been genetically stable during 3 years of continuous propagation (FIG. 1D).

### Methods:

### Small-Scale Spirulina Culturing

Spirulina strains were grown in liquid culture using SOT media. For antibiotic selection, media was supplemented with 70-100 μg/mL kanamycin or 2.5-5 μg/mL spectinomycin. Culture volumes ranged from 3-100 mL. In preparing strains for transformation or downstream processing, cultures were grown in Multitron incubators at 35° C., 0.5% CO₂, 110-150 μEi of light, and shaking at 120-270 rpm depending on culture volume. Long-term cultures were maintained by incubation in Innova incubators at 30° C., atmospheric CO₂, 50-110 μEi of light, and shaking at 120 rpm.

### Design of Integrating Vectors for Spirulina Transformation

For each genomic loci targeted for integration, PCR primers with 18-20 bp overlapping sequence with a vector backbone were designed to amplify 1-1.5 kb DNA fragments from the 5′- and 3′-regions flanking the locus. These regions represented the left homology arms (LHA) and right homology arms (RHA) respectively. Gel purified fragments were assembled with the linearized backbone vector, which contained a p15 origin and an E. coli ampicillin resistance marker, by Gibson assembly. Markers for antibiotic resistance in spirulina were cloned in between the two homology arms of the plasmid.

Homology arms of the disclosure can target any region of spirulina. The Spirulina genome comprises 6630 potential protein coding genes, 49 RNA genes which consist of two sets of rRNA genes, 40 tRNA genes which code for tRNA, tmRNA, β subunit of RNAse P and signal-recognition particle RNA. In some embodiments, homology arms target a gene involved in metabolism, photosynthesis, and combinations thereof. In some embodiments, homology arms bind a protein-encoding gene. In some embodiments, homology arms bind a non-protein-encoding gene.

### Transformation of Spirulina (Natural Method)

Spirulina cultures were grown for 3 days in Innova to reach an OD₇₀₀ of 0.5-1. A volume of 50 mL of cells were harvested by centrifugation for 10 min at 1,600×rcf. Cells were washed once with SOT media at room temperature. Cells were resuspended in 2 mL of SOT. A 30 μL aliquot of cells were mixed with 300 ng of plasmid DNA and incubated at room temperature for 3 h. Samples were transferred to 0.6 mL of SOT media in 13 mL round bottom tubes and incubated overnight at 25-30° C. in 60-70 μEi of fluorescent light on a light rack. Each tube received 2.4 mL of SOT with appropriate antibiotics and was incubated in Multitron to start the selection. For the first 20-30 days, culture medium was changed every 3 to 5 days. After 30 days, when the transformants were robustly growing, cells were diluted every 3-5 days to facilitate segregation.

### Genotyping of Transformed Spirulina

Genomic DNA was prepared from spirulina cells by digestion with proteinase K. Briefly, OD₇₀₀ of cells was washed once with sterilized water. A 30 μL sample of cell pellet was mixed with 120 μL of buffer EB (10 mM Tris-Cl, pH 8.5). Proteinase K was added to the samples at a final concentration of 0.2 mg/mL. Samples were incubated at 56° C. for 1 h followed by 95° C. for 10 min to deactivate proteinase K. Samples were centrifuged briefly to pellet cell debris. A 1 μL sample of the supernatant was used per genotyping PCR reaction. Specific integration of the transgenic cassette was determined by separate PCRs for each homology arm. For each PCR, one primer annealed to a genomic sequence outside the homology arm and the other annealed to a region within the transgene. Segregation of the chromosome was assessed using a primer pair annealing to regions within the RHA and LHA. The segregation PCR yielded fragments of two different size: one from the wild-type allele and the other from the transgenic allele. Strains were considered fully segregated when no wild-type allele amplicon was observed.

To verify the sequence of the transgene, PCR was performed with the genomic DNA to amplify the fragments which includes the transgene, the homology arms, and 500 bp flanking each homology arm. PCR products were separated by electrophoresis on an agarose gel and the amplified bands were gel extracted using the Qiagen Gel Extraction kit. The purified PCR products were sequenced to verify the integrated gene and the surrounding sequences.

To exclude the possibility of cross-contamination with other strains, PCR was performed to check other loci that have been used for integration of exogenous genes. PCR of genomic DNA using locus specific primers was performed and fragment size was analyzed by agarose gel electrophoresis. DNA fragments were gel extracted and characterized by Sanger sequencing. A strain was considered free of other spirulina strains if only wild-type loci were observed.

### Transformation of Barcoded Integrating Plasmids

To evaluate the number of individual successful integration events per transformation, a library of DNA barcodes was transformed into spirulina and quantified by next-generation sequencing (NGS). Briefly, a 19 N barcode was cloned adjacent to an antibiotic marker (aadA) in a plasmid containing homology arms for integration at the NS1 locus. The barcode library was estimated to contain>8×10⁷ transformants. The DNA barcode library was transformed into strain SP003 in triplicate, following the transformation method described above, and cultured with streptomycin. Spirulina samples were collected 22 and 28 days after transformation. Genomic DNA was extracted from spirulina and used in a PCR reaction to prepare ˜320 bp amplicons of the barcoded regions for NGS analysis on a MiSeq (Illumina). Sequencing reads were filtered for quality and analyzed to minimize the false positives. Counting only barcodes that were 1) present at both timepoints within a replicate, 2) unique to each replicate, and 3) observed more than 30 times within a sample yielded an estimated minimal number of integration events of ˜100-300.

### Isolation of Single Spirulina Filaments

From an actively growing spirulina culture, 200-500 filaments were spread on a SOT plate. The cells were allowed to settle on the plate for 1-2 h and examined under a dissection microscope. Well separated single filaments were picked with a 1 mL pipette tip and transferred in 3 mL SOT with appropriate antibiotics in around bottom tube. Single filaments were cultured in Innova for 15 days to propagate.

### Determination of Transgene Copy Number

To assess the copy number of an integrated transgene, three sets of primer/TaqMan probe pairs were designed to target 3 regions: an endogenous spirulina gene present at single locus (cpcB), a promoter region present at both an endogenous and transgenic locus (i.e. two chromosomal copies), and an exogenous region unique to the transgene. A synthetic g-block containing the 3 target loci plus flanking sequences was purchased from IDT as a calibrator. Real-time PCR was performed with the above primer/probes using genomic DNA from the transgenic spirulina strain and the g-block as templates. As controls, the parental spirulina strain and a second transgenic strain lacking template for the transgene-specific probes were tested. The relative copy number of the integrated transgene was calculated as the fold difference between the transgene and endogenous gene with ΔΔCt method. The experiment was repeated 5 times with three separate preparations of genomic DNA. The expected abundance ratio for the endogenous gene, promoter, and exogenous gene was 1:2:1.

### Axenic Strain Isolation

To establish axenic spirulina strains, cells were grown to a density of 0.5-1 OD₇₅₀ in Innova with proper antibiotics. Cells were pelleted from 5 mL cultures by centrifugation for 10 minutes at 1600×rcf. To maintain sterility, the following steps were performed in a laminar hood. The cell pellet was resuspended in 1 mL SOT and transferred to a 10 μm filter pre-wetted with SOT media. Media was removed by gravity filtration. Cells were washed with successive 1 mL aliquots of SOT media until at least 200 mL of total media had pass through the filter. The remaining cells were resuspended with 0.5 mL of SOT and transferred to a sterile Eppendorf tube. Filaments were counted under a microscope as above, and 200-500 filaments were spread on a SOT plate. Single filaments were isolated as above. After 10-14 days, 10 μL of culture was spread on LB plates without antibiotics. Plates were incubated for 3-5 days in a 37° C. incubator. Filament cultures free of contaminants on the LB agar plates were then seeded in 10 mL SOT with 2.5 g/L dextrose at a density of 0.1 OD₇₅₀. Cultures were grown in an incubator for 3 days. A 100 μl sample of the culture was plated on LB agar plates without antibiotics and incubated at 37° C. for 5 days. Cultures with no contaminants observed on either set of LB plates were considered as axenic.

### Culturing of Non-Spirulina Microbes

To culture non-spirulina microbes, a flask of spirulina culture was placed on bench for 3-h to allow the spirulina cells to settle down at the bottom of the flask. A 100 μL sample of supernatant was carefully pipetted and transferred to LB or mixed LB/SOT agar plates. Plates were incubated at 25-30° C. on a light rack (60-70 μEi of light) for 5-7 days. Single colonies were streaked on the new plates for 5-10 rounds. Cells from single colonies were spread on fresh plates to propagate for further experiments.

### Example 2—Induction of Spirulina Competence

We determined that transformation competence could be induced in spirulina by co-cultivation with specific co-culturing microorganisms. Spirulina UTEX LB1926 obtained from the UTEX algae culture collection was not axenic. Both direct microscopic examination and seeding of the culture medium onto solid LB+SOT plates revealed the presence of contaminating microorganisms. Axenic strains of spirulina UTEX LB126 were generated using micromanipulation to pick single filaments. 8 of 11 single filaments produced axenic cultures as verified by microscopic examination and confirmed by negative results after cultivation on LB+SOT plates. 3 of 11 single filament-derived strains remained non-axenic. When exposed to 2 different integrating DNA vectors, all 3 xenic filament-derived strains remained naturally transformable, whereas none of the 8 axenic cultures were transformable, suggesting that competence for transformation was associated with the presence of other microorganisms.

Microorganisms found in the original xenic strain of UTEX LB1926 were streaked to single colonies. 12 isolated colonies were individually co-cultured in liquid medium with an axenic UTEX LB1926 strain. The axenic spirulina strain remained non-transformable, but the spirulina in all 12 co-cultures became competent for natural transformation. Microorganisms that induced competence in these cultures were identified by sequencing 23S and 16S chromosomal DNA as belonging to the genera Sphingomonas and Microcella. Similar results were obtained for NIES-39. Therefore, we used xenic strains for genetic transformation and derived axenic variants for subsequent protein production.

### Kraken Taxonomic Sequence Classification System Analysis

To identity potential co-culturing microorganisms, a Kraken analysis was completed on spirulina strain SP205. Exemplary co-culturing microorganisms comprise: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus and Oscillatoria, Koinonema, Oxynema, Planktothrix, and Microcystis.

Identification of Non-Spirulina Bacteria from Spirulina Cultures

To culture non-spirulina microbes, a flask of spirulina culture was placed on bench for 3-h to allow the spirulina cells to settle down at the bottom of the flask. A 100 μL sample of spirulina-conditioned media was transferred to either LB agar, or mixed LB/SOT agar plates. Plates were incubated at 25-30° C. on a light rack (60-70 μEi of light) for 5-7 days. Genomic DNA was extracted from bacterial samples following the genomic DNA extraction method described above. Highly conserved and degenerate 16S and 23S rRNA PCR primers and methods (Hunt (2006); Klindworth (2012)) were used to amplify DNA from samples derived from both LB and LB/SOT plates. PCR product libraries were subcloned and sequenced, revealing that Sphingomonas was the dominant LB-derived isolate and Microcella was the dominant LB/SOT isolate.

### Example 3—a Markerless Method for Engineering of Spirulina

The site of chromosomal integration was dictated by the homology arms flanking the transforming genes. A construct containing a single homology arm was ineffective at transformation; this indicated that integration occurred primarily by double crossover events, as is typical for natural transformation. The homology arms were 1-1.5 kb in size, though efficient integration was observed with a 118 bp homology arm. To date we have evaluated 11 sites of integration, of which 6 were shown to accommodate an insertion with no apparent deficit in a standard growth regimen as they are considered to encode non-essential proteins.

TABLE 1
Exemplary insertion sites. Bolded sequences denote the insertion site with 200 bp
flanking sequence. The bolded sequence was replaced with integrated sequence encoding a
transgene.

| | Location | |
|---|---|---|
| | (specified | |
| | within | |
| | arthrospira | |
| | platensis | |
| SEQ | NIES39 | |
| ID | reference | |
| NO | genome) | Sequence |
| 1 | NIES39_ | acatgatcaagtagttgtttaacctcgctctgtaggctttgtgtagtatcttctggcttaggtgctgcgcca |
| | Q01220 | ttagtgcggcgaacagcttgaccattagtgcgggaagatttgagacttttagctgtggaagtaggttga |
| | | ggttgagaagaaccgccgccccaattttgaacagccgtcgccacatcaatacccgtagtttggcgca |
| | | actcttccaaaaacgccgccattttagaagctgtattccctttattagcatcaacaatagtcacat |
| | | tatcaacagtaatttcaggaacagtagaagccatagtttttagtaagacttccaacttctgataa |
| | | aggaaaatttccctagcattaggaccggctgctttccaagactcagccagtcgtcgtgtcccttcag |
| | | cttgtgctttcccatcttcaataatttgggcagcatccccccgcgccctagcgatcgcctttttacattccg |
| | | cctctgctggtgcgaccacatcagcttgtagttgctgttctacctgcttaatcctttctttttgaacagagac |
| | | ctcagcctgag |
| 2 | NIES39_ | ccttaaccccctaagccctatgtctgacacccagtcaccccaaccgccttagtcggtattccccactgg |
| | C03030 | gtgggcgatacacgatttcccggaaaaattgaaaaaaaattttgtggcaggtgatatgatttgcgatcg |
| | | aggtgggtaagttagaactacagaaaacaaataacctcatttcaccccgaggagaacgaattatgaa |
| | | aactgaacttaaagccaaattcctgcaacatatctctcagaaaaaaagagacggtggtttcacc |
| | | ttaatcgaactgttagttgtaattatcattatcggtattttgtcggcgatcgctctgccttccttctta |
| | | aaccaagctaacaaagccagggagtccgaagctaaaacatacgttggttcaatcaaccgggc |
| | | tcaacaagcgcgtttgatggaaactggcgagtttactacctcaataactcaactgggtctgggtg |
| | | tcagcagcgcaacagcaaactacacttataatctactcgctaatgcttctggtgcggtggttaat |
| | | gccaacggtatagctaatctccgcgactatggtggtctggtctgggtaggtactacagcaggag |
| | | gggagcgtaccactttggctaaactgtgcgaagcgcctgatgcagaaaccgaggcaaagtttg |
| | | cagcgggcgctaatagttgtggtacttttagagaaatttaacagataatctagcagtttaggtttcag |
| | | atggagtcagctagggtctttaaccccatcgtcaactataagatggagagatggcatacggtgacact |
| | | gttaaagccataactatattgctcttgttgataaaatcagaaactgaggccacttgagagtattgtaaata |
| | | ggtagtacagtagtgctacctattttgttttaaa |
| 3 | NIES39_ | tctggcccttaattgtagtcgatcgcccagaatattataccatccctttaggcgtggcaaatctagccgg |
| | D01040 | aactttttctctcgattggcggatagtcgccgccggttccgtcatttctatcgctcctgtgttactactatttt |
| | | taatcgtacaacggtatattgtgcctacagatacagctacgggggttaaaggttgaggattattaatct |
| | | cagtcctaataatcaaaatcatatccaccaggcggcgactttattagtcgcagaatttcgggaa |
| | | aattggccgaatgcttggccgacttatgaacgtggtttagccgaggttatggagtcttttggcga |
| | | cgatagagttaatctagtcgccgtcgatgaaaatgataatcttctaggctggattggtggtatta |
| | | gccaatatcaaggccatgtttgggaattacatcctttggtagtcaaaagcgattatcaagggtta |
| | | ggaatagggcgaaaattggtcgccaatttagaggattatgtccgctcacaaggagggttaacct |
| | | tatggttgggtaccgatgacgaaaataatttaacctccctatctggtgtcgaattatacccccatt |
| | | ttttggaaaatattgctaacataaaaaaccatggtcgtcatccctatgaattttatcagaaatgcg |
| | | gttttgtgattatgggagtagtccccgatgctaacggtatcgggaaacctgatattttgatggcta |
| | | aatctctgagaattgacaatcatcctaaatccaactgaggaatcatgaatcatcccacaactaagc |
| | | atcaaaaaattacatcctgttactggggaatttctgaactacctgggttagatacaggtaatattaaactc |
| | | ctgaatcaatgtggtattgaaaacacccagcaactgctcacgcgcgggagtaataatgctaataaaat |
| | | cgccttatctaaccaactaggtatcaacatcag |
| 4 | NIES39_ | gtatcagcgttaactatggtgcaatttatcccttgctgaaacgtttggaagaacggggatatattcaagtt |
| | A04200- | atcctagaagaggcaggggacgcgggttcgagtcggaaggtttatggtattaccgccgccggacgt |
| | A04210 | gagcgatggttatacaaaatgctagaacagccgcaagagagttgggttaaaagccgttctaggtttct |
| | | aattaagtatttcttttttagtgacttaaatatacacaatcgcctgaaattgttagaatatcgcctc |
| | | agagcttgtcagcaaaggatggcatatctgaaccagaaagaggttgaatatgtgccgattgatt |
| | | cctatcaatcagatatctggcaaagggcgaagtttttgttagattcagagtctaaatggctcgaa |
| | | aaaacctacgataatttgcaacaagaactagggggtggtgattctctatcttcagacagatcac |
| | | aattgtgaatagccgagatctacaatggcagtagactagggaattgacagtaaaattttgccgc |
| | | caacaattataatcatactacaatttattatgtcttatccatcatcaacagaatcatcaaactcga |
| | | attatcatcagtcggtcgtggaagttgaggggagtggtgaacattcgccaatagaaaaccaaa |
| | | atcagatagaaagcgatcgccattcggaatcagaacctagtaataaatccaacaattgggtat |
| | | cagcgaccgtaggaatattactagccctaggagtaggctttgctgggggtcagtggtggtactc |
| | | cagtaaagcgcaagcggacactaccccagcggcggcgagtaattccccccaaagaccgagg |
| | | ggagttccggtgagagtcaccagcatagaaactaggaactttcaggatgctggggaatttgtgggga |
| | | ctctggaagcgcgggagtcggtggaagtaagatcggaaatagaagggagaatcacgcaagtatatg |
| | | ttaggtcaggggagatggtaaatcaagggcaggcgatcgcccgtttaagaagtgatgacctagaagc |
| | | gcaactac |
| 5 | NIES39_ | ggctttttgcgatcgcatcttttgccgtcggtgctttcgccattttaccctatttggtgtggcgaaaacctaa |
| | D00590 - | ccccgaattttctggggttcccaatctcttcatccgcatttgggatagcaaattcacagctttagtcttaat |
| | D00600 | gttaggggcgatcgtcctagttttttatggtatcactcagggagattggcgtgactttatctatcaatgg |
| | | caaaatagccgttttattcacgttatgagcctcgacttctgtttattatcagtattatttcccgcctt |
| | | attaggggatgatatcgcgcggcgcggcaaccaggctgcgacttggttatggttggtgacactc |
| | | attcccctattcggacctttactatatctctgtgtgcgctctcccctgtgcgatcgtctttgagtcac |
| | | catccgtagttttcatcaaagcctcattaattcagttagaaaaccgcaagaatccgcaggcagt |
| | | agtgtaaaatctcaataataggtatcagggtcaacccaagagttaggctaaaattcacctccaa |
| | | acctgatacaatctcctcatcaaattgcgggatcgtcctatgaacaaaacactatggaaaacta |
| | | gcgcatctctaccgctgctattattatttgtactcatcaatgcttgttcagcagaacctcaagccattggtc |
| | | gcccacccgtccccgtcaagatagaaaacgtagatactaccgatgttcaatccagcagcgaatataat |
| | | gccagggttgaaggtatcgaaaactctatcatcagaccccgagttagtggattagtaaa |
| 6 | NIES39_ | gtttgccgcattttaattaatcagcaaacccatgaacaagtcagctatcaatgtctaactgaatatatcgg |
| | E03960 - | tagcattcacctcaaaggtcgcactcaacctattaatgtttatcaaattaacaatccttagtctgtattaaat |
| | E03970 | ctttccatctattctgtcaactgtcctaggtagttatgaatagccacagcttttgatgaatctcctcgcccc |
| | | cagatgtaattcacggcggagacagctccgcacccccacccacattatccccccctcccagag |
| | | ggggacaagataaggagtttcatgattcaccgcgacaccacattaactttatcgccatctttaag |
| | | ataacttgcaccagcgatcgcaatgcgatcgcctacatttaaaccctgtataatttctatactcgc |
| | | ctccgataaatcgcctgttaaacggccttgaagtcttcccacttccacagttcgcgcttccaccgt |
| | | atcatcaggtaatagacgataaacaatagaactaccgtcagcctggggaacgatggccctagc |
| | | gggaaccttcattccttgtacagtagtagtagtaatcgccgcccgcagaaacattcccggacgc |
| | | aagaaagaatcacgaatagcccccaagggaggcaggtcaattttcacagtagcttgtcggcttt |
| | | gaggatcaatcagaggcgcaatttctcgcaccacccctcgcacctgcaaattttgatcagcatc |
| | | agaagtcacagtaacagcagcaccaattctcacctgtgataactgagtttctggaaccttcgcgtgt |
| | | agttctaactgattttccgcaataatcgaaaacagttgttgattaccagtaacatcccccactcgcgctgt |
| | | ccgttgggcgacaatgccactcataggcgcacggactacagtttgttcccgttgagtttccaactggcg |
| | | aacccgcgcctgggcgctacgcacctcagc |
| 7 | NIES39_ | atgaaatgtgaaggttctccggtcgtagaaacacacaattgggatattcctgctgtagcgattgcatgc |
| | K04040- | aaaacctatttagataagacgatgctgcaagatatttctacggcggctgaacaactcaagtataaaaat |
| | K04050 | cccaatgctatgtatattgtagtggcagaatggctcaaacttactgactcggtgaatctgagaaaatat |
| | | aaaattgatcaaatttatattttgcgaaaacaaaaaaatactgatagagaatttagatatcttga |
| | | gagttatcagaaaaatcctatatactctgatgtagtacaacatctatttctgaaagtacgagagt |
| | | ttctgatttccgactgggagggaggaatatcagatggtttaaatcgtggttttttactctaaaatct |
| | | tggctatgacaattcctctggcttccccgagtccctgccctaattatcccatatcattattaaactc |
| | | gcaaggaggtgaatctttccgcgtttgtatgctatgatatgctacaattcagactatacttttctcc |
| | | cttgacaacccaaaccgattagattagatgactgtaaattaatcaataaattatggctaaaaat |
| | | agttttaatctttccgaatggtcgattcgacaacctgttcccactttggtgctgtttttgatcctgac |
| | | gctggtggggttgatgtctttttttcaactgggaattgatgatactcctaatatagatattccggcg |
| | | gttacggtgacagttacacaaccgggtgcgggtcctaatgaattggaaaatcaggtgacaaaa |
| | | caggtcgaagatgcgatcgccggattaggaaatattgaccaacttacctctacagtcagagac |
| | | ggagtatctaccactaccgtgagttttgttttgggaaccgatagcgatcgcgctactaatgatgtcc |
| | | gtaatgcgatcgctcaaattcgccaaagcctaccccaagacattaacgatcctattgtacagcgtctgc |
| | | gatttgcggggggtcgattatgacctatgcggtaacatcacccacccgctcagtagaagaacttagc |
| | | gatcttgtcgatcgcactattagccgcgccttac |
| 8 | NIES39_ | ctttgagccctggcgcattccccaggatctatgctttgaccacgatcgcattttacaagactatctgcaat |
| | A01980 | ttcgccattttggactccgacccaggctaatgtgagtcagtgaacacccgactacccccaactcgcttc |
| | | ttctcagaaaaagagagtaagagacagaggtggcccactaagtatgggggtaatatccacttactca |
| | | gcaccggaattgggttctaagccccactgatacttgagaaagtgctgatacatattctccctaac |
| | | caacatttgttcgtaaagatcaaccaaaaactcctgggcttgttcccggctcatttggtttacctg |
| | | cattctaaaggaggacaggttaaattgttgttccaaagataagctactagattccatgatgacgt |
| | | gaactccttaaaagtgaatttgggttaattttctaggatggccacccttgtcccggaaattattacga |
| | | attataacaattatttgacaaagtgcctaccttaatatacccaaatctttggatttaccataacagatctagc |
| | | cctaaaatttgtcatccacgatacgagatgactggatagttgggaaaaaactatggctgtggggggat |
| | | aagtggcaataattaaccgaaactgagaggatca |
| 9 | NIES39_ | gtagcaagcgttttggctacctggtcgagtttagcgatactcatagtcttttccaaaatccccaacaaga |
| | A02690 | agccacacaacaatatgttagtggtcgctttggttaagcataacctattcccaagaggtcagaggctac |
| | | ttaacttaatcagccctttctgacctccctctggttgtggtgattggttgatggtgttttgaggctatgcgc |
| | | ctttcatccagtttttcagtaaattatccttgaccatcccttggcgaaatacctcaattaaatactc |
| | | ctgagcttcttctaaacttaggtgtttgacttgttcctggagaaccttgagcttgaattgttgctcta |
| | | tgcttagatttcctggggtttgcattgttcgctcctcctctgaaattctaggggtgagagtacggttttg |
| | | ggtcaaaccgtaacttttgtttacctggaatgtaaattatcgtaacatacacttgacaatctgtccatgatg |
| | | gacatttaggcactaatctatttaacaaaactttggattttccgatctgatagccacagactcagccctaa |
| | | ctgccataaaataaacggatagcc |
| 10 | NIES39_ | gatgctaatatgttagcaaaatttcggctccctgtcaacccctgcagccgggcggatttgcgtgttgac |
| | J05970 | gaaaaaatcaaaaaagtgatcgggtgcgtcatagcaattaataatgatttcccgatggggaaatgagc |
| | | ggactgacgcaccaaaattgtggttgagaaattcccggcttaattaaccaggaagttgctcaactaag |
| | | cgccaccgcgcagcttttcgagaacggtgcgatcttggagagtggaagtatccccagaaatctc |
| | | ctgacccgaagcaagcgatcgcaataaacgccgcataattttacccgaacgagtcttaggcaa |
| | | atcatcagaaaagcgaatttccccgggacgcgccaaggctccaatttcattaacaacgtgctgc |
| | | ttcaactctttttccaaggcttcatcaggttggcgatcgccttcaagagtaacaaaagccacaatt |
| | | tcctcacccttgacttcatcaggacggccaaccaccgccgcctcagccacagccggatgagaa |
| | | accaaagccgactcaacctccatagtccccaagcgatgaccagaaacattaatcacatcatca |
| | | acccggcccatcacccagaaatagccatcctcatctcgatgagcgccatcaccagcgaagtag |
| | | acataatcgccatttttaggtcgtagatactcccaataagtacggcggaagcgctccgggtcgc |
| | | cataaacagtccgcatcatacccggccacgggtgacgcaccaccaaatatcccccagaattatt |
| | | agtaacaggttccccttccgtatcaaccacatcagcaataattccagggaaaggcaaagtagc |
| | | cgaacccggcttagtaggagtcgcaccaggaagaggagtaatcataaaaccccccgtttccgt |
| | | ttgccaccaagtatcaacaatgggacattttccttgaccaatgacgcggttataccaaatccagg |
| | | cttctgggttaatcggttctcccaccgtacccaaaatccgcaacgaagacaaatcacgagcatt |
| | | agggtgtctttcacccattttcatcaaagctcgaatagcagtaggagcagtatagaaaatagtc |
| | | acgccatacttttctaccacatcccacagacaaccgggattagaagcacggggagccccttcat |
| | | acatcaaagttgtcgccccattggacaaaggcccatagacaatataactatggccagtaatcca |
| | | acccacatcagcggtacaccagtaaacatcagtatcctgtaaatcaaaagcccactgattagta |
| | | atgtgactgtaaagattatagccaccagtagtgtgtacaactcccttgggtttgccagtagtccc |
| | | gctagtgtaaaggataaacagcatatcctcgctatccatttcttcggcgggacattgcccggaa |
| | | gcattttgttgcaagtcatgccaccagtggtcccgtccgggttccatctggattttttgctcagtgc |
| | | gctgtacgaccaaaacgttatcaacactaggcgcaccagcttgtaaagccttatctacctgttct |
| | | tttaggggtacgatcgcatctttgcgccaaccaccatcagcagtaatcaccaatttagcttcggc |
| | | atcttccaggcggtcctttagagcctcggcactgaaaccgccaaaaatgacggtatggggtgcg |
| | | ccaatcctcgcacaagccaacatggcgatcgccgcctctggtatcatgggcatataaatcccaa |
| | | cgcgatcgccttttttgactcccaactgtttaatcacatttgccatttggcaaacttcccgatgtag |
| | | ttgggaataggtcagagtccgagagtctcccggttccccttcccaaattaaagccgccttattttt |
| | | gcgccaggtagtcaaatggcgatcgaggcaattataggaaatattaattttgccccccacaaac |
| | | cacttcgcaaaaggtggctgccagtccagcacctgatcccattttttaaaccagtgcaactcattt |
| | | tccgctaattctgcccaaaacgcttccgggttagctttggctttctcgtagagttcccggtactctt |
| | | ccatgctcttaatccgggcctgtttggagaagtcctcggacggattaaataaccgtttctcgtgta |
| | | aaattgattcgatcgtcggttgtgacatagtgtgatggtaactttaactacaaagtgaagccattataa |
| | | ccattttaggcctagattaaactctaagaatctttacttttttttgagcagactgctaactagctaactcctcc |
| | | ccatgtcaaaccgattaaaatcagttgtcaacttctaagtaactatgcctcctcaactcattatccgtcctg |
| | | ctactgttgatga |

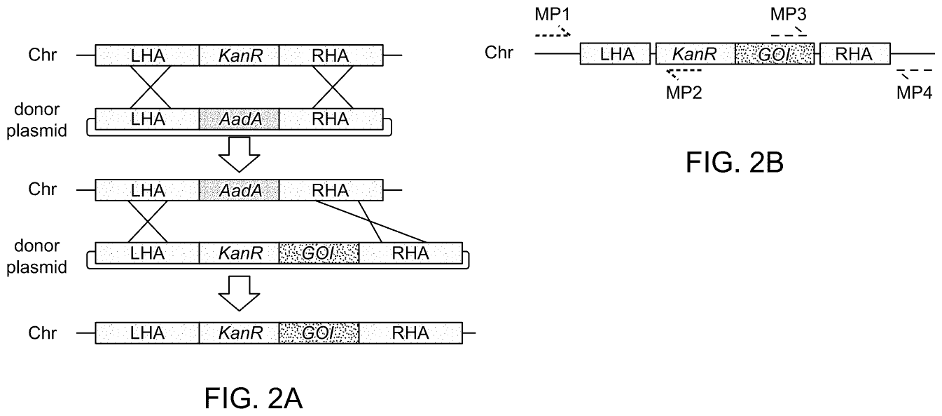

One selected insertion site corresponded to NCBI reference sequence: WP 006618409.1, that encodes a putative acetyl transferase. We fortuitously observed that deletion of this locus rendered spirulina kanamycin sensitive. Kanamycin resistance encoded by this natural spirulina gene presented an opportunity for development of a markerless genetic engineering strategy. The first step used homologous recombination to precisely replace this gene with an exogenous gene (aadA) encoding resistance to spectinomycin. The resulting strain, SP205, was kanamycin sensitive and spectinomycin resistant, but otherwise identical to the parental strain. In the second step the natural spirulina kanamycin-resistance gene was joined to a gene of interest and precisely re-introduced into its original location in the genome of SP205 by replacement of the exogenous aadA gene. The resulting strain had kanamycin resistance restored, expressed the gene of interest, and again was spectinomycin sensitive. This strain contained the gene of interest integrated into the spirulina chromosome at a pre-selected, defined location and contained no other exogenous genetic information (FIG. 2).

To date we have demonstrated introduction of a variety of exogenous DNA vectors into the spirulina genome, including single genes, genes in tandem, and operons, as well as sequential engineering of different insertion sites has been demonstrated. The largest transforming DNA cassette introduced was 4.5 Kb and contained a 5-gene C-phycocyanin operon from a thermophilic cyanobacterium. We demonstrated intracellular expression of a diverse group of exogenous proteins, including bioactive peptides, antibodies, protein pigments, and enzymes. Self-assembling nanoparticles derived from bacteriophage or viral capsid proteins for multivalent display of vaccine antigens have also proven readily expressible. The amount of an exogenous protein expressed using a strong, constitutive promoter derived from the C-phycocyanin locus (P_{cpc600}) was as much as 20% of total soluble protein.

Methods

### Markerless Strain Engineering

To create a platform for markerless integration, a parental strain containing a recombinant, non-native antibiotic marker was first generated. An integrating plasmid bearing homology arms for the D01030 (kanR) locus flanking an aadA gene for streptomycin resistance was transformed into wild-type spirulina. The integrating vector was designed to precisely replace the ORF of D01030 with the sequence for aadA. This vector was transformed into both UTEX (SP3) and NIES (SP7) spirulina strains, generating strains SP205 and SP207 respectively. After transformation, strains were propagated for 2 months and confirmed to be fully segregated by genotyping. The strains were also challenged with kanamycin to demonstrate the loss of native kanamycin resistance.

### Verification of Markerless Spirulina Strains

Clonal isolates of fully segregated strains were verified as follows: 1) qPCR to demonstrate a single transgene per genome (see above); 2) sequencing of chromosomal DNA to verify the absence of mutations in the homology arms and inserted gene(s) (see above); 3) PCR to demonstrate loss of parental integration locus allele and complete segregation to homozygosity of the transgene (see above); 4) chromosome DNA sequence of the 16S rDNA locus to verify strain identity as Arthrospira platensis; 5) sequencing of alternative insertion sites in chromosomal DNA to verify lack of strain contamination with other engineered spirulina strains (see above); 6) PCR to demonstrate absence of the integrating DNA vector backbone, which should be eliminated during integration by homologous recombination (see below); and 7) verification of spectinomycin sensitivity and kanamycin resistance.

The vector backbone sequences outside of the homology arms should not integrate into the genome and thus be absent from genomic spirulina DNA. To exclude the possibility of non-specific integration of the vector backbone DNA, PCR was performed with primer pairs targeting the ampicillin resistance gene and E. coli origin of replication. At no point have these fragments been observed in spirulina, suggesting that there is no integration of the vector outside of the homology arms.

### Construction of Markerless Transgenes for Spirulina Integration

To ease cloning of transgenes into spirulina, a standardized vector was built for markerless integration. This “destination” vector included integrating homology arms for the KanR locus flanking an ORF for the native KanR gene and a terminator. The antibiotic marker was followed by a recombinant promoter/terminator pair for transgene expression. The promoter/terminator pair consisted of a constitutively active, native A. platensis promoter (600 bp upstream of the cpcB gene; named P_{cpc600}) and the terminator of the E. coli ribosomal RNA gene rrnB (named TrrnB). A pair of BsaI restriction endonuclease sites between the promoter terminator pair was used for Golden Gate cloning of protein coding sequences for transgenic expression. Protein coding sequences with compatible BsaI sites were purchased from IDT and cloned into the “destination” vector using a Golden Gate Assembly Kit (NEB). Plasmid DNA was purified from E. coli by QIAprep Spin Miniprep Kit (Qiagen) and transformed into the spirulina strain SP205. The product of integration of this construct is genetically identical to the wild-type KanR locus, excepting the transgene (i.e., no non-native antibiotic markers are present).

Purification of Recombinant Protein from Spirulina

Recombinant aa682 was purified from spirulina by immobilized metal affinity chromatography (IMAC). Briefly, a 10 mL pellet of spirulina cells from strain SP1182 was collected from 2 liters of culture by centrifugation. Pellet was resuspended to a total volume of 35 mL with lysis buffer (50 mM sodium phosphate buffer pH 8.0, 500 mM NaCl, 20 mM imidazole) supplemented with Pierce Protease Inhibitor Minitablets (Thermo Scientific) and 1 mM phenylmethylsulfonyl fluoride (PMSF). The resuspension was passed through a French press cell twice to lyse the cells. Samples were kept on ice throughout. The insoluble fraction was pelleted by centrifugation at 5,000×rcf for 30 min. The partially clarified lysate was mixed with 2 mL of washed HisPur Ni-NTA Resin (Thermo Scientific) and incubated at 4° C. with gently rocking for 2 h. The resin was gently pelleted by centrifugation at 500×rcf for 1 min., supernatant discarded, and resin resuspended in fresh lysis buffer. This process was repeated until the supernatant was clear. The resin was collected in a small column by gravity filtration, washed with 20 mL of lysis buffer, and spirulina-expressed aa682 was eluted with lysis buffer containing 200 mM imidazole. Purified aa682 was further polished by separation on a Superdex 75 Prep Grade column on an ÄKTA Pure, yielding a single band by SDS-PAGE electrophoresis.

### Preparation of Spirulina Extracts for Analysis of Soluble Protein

Soluble extracts from spray dried spirulina samples were prepared by a flash-freeze protocol. Dried spirulina biomass was resuspended in PBS containing Pierce Protease Inhibitor minitablets and 1 mM PMSF at a biomass concentration of 10-40 mg/mL in 1.7 mL Eppendorf tubes. Samples were mixed by inversion to resuspend biomass powder and flash frozen in liquid nitrogen for 2 min. Resuspensions were transferred to a 37° C. water bath for 2-3 min. Samples were well mixed by inversion once thawed. The flash-freeze procedure was repeated 2 additional times. Biomass samples were then centrifuged at 15,000×rcf at 4C for 30 min, and the soluble fraction was transferred to a separate tube for downstream applications.

### Expression Analysis of Recombinant Proteins in Spirulina

Recombinant protein expression in spirulina was measured by capillary electrophoresis immunoassay (CEIA) using a Jess instrument (ProteinSimple). The Jess system was run as recommended by the manufacturer. Briefly, dried biomass samples were diluted to a concentration of 0.2 mg/mL using water and a 5× master mix prepared from an EZ Standard Pack 1 (Bio-Techne). Purified protein controls used to generate standard curves were typically loaded at a range of concentrations from 0.5-20 μg/mL. A 12-230 kDa Jess/Wes Separation Module (ProteinSimple) was used and 3 μL of each sample was loaded for 9 s. A mouse anti-His-tag antibody (GenScript) was diluted 1:100 and used as the primary detection antibody. An anti-mouse HRP-conjugated secondary antibody (ProteinSimple) was primarily used for chemiluminescent detection; fluorescently labelled anti-mouse antibodies (ProteinSimple) for IR or NIR fluorescence detection were used for some experiments. Data analysis was performed using the Protein Simple Compass software.

Expression, Purification, and Biotinylation of E. coli Expressed Proteins

Recombinant C. jejuni flagellin was expressed and purified from E. coli. A region of flaA (Sequence ID: WP 178888959.1) predicted to be soluble and exposed on the surface of flagella (amino acids 177-482) was cloned onto the C-terminus of MBP in a pET28b E. coli expression vector. The vector was transformed into BL-21(DE3) cells were grown overnight at 37° C. on agar plates with kanamycin, and a single colony was used to inoculate a culture of LB media containing kanamycin. Cells were grown overnight with shaking at 225 rpm at 37° C., back-diluted to OD₆₀₀ of 0.05, and grown at 37° C. until the cells reached mid-log phase (OD₆₀₀=0.4-0.6). Cells were induced with IPTG and incubated with shaking at 16° C. overnight. The following day, the cells were pelleted by centrifugation at 3,500 for 20 min at 4° C., resuspended in 30 mL of lysis buffer containing protease inhibitors, and lysed with a Q700 Sonicator (Qsonica). The MBP-flaA fusion was purified from the clarified lysate using Amylose Resin (NEB) per the manufacturer's recommendations, and purified protein was aliquoted and stored at −80° C. Biotinylated MBP-flaA protein was prepared using an EZ-Link NGS-PEG4-Biotin kit (Thermo Scientific) following the manufacturer's guidelines. VHHs expressed in E. coli used similar expression vectors and bacterial cells lines. Culturing, induction, and lysis of E. coli expressing VHHs followed the same protocol as with flaA expression. Purification of the VHHs from lysates was performed by IMAC, following the purification protocol described for aa682. The RBD antigen used with VHH-72 was a kindly provided by the Roland Strong (Fred Hutchinson Cancer Institute).

### Binding Assays for Spirulina-Expressed VHHs

The EC₅₀ binding activity of VHHs as purified protein and in spirulina lysate was measured by ELISA. High-binding 96-well plates (Greiner Bio-one or NUNC MaxiSorp) were coated with antigen by adding 100 μL of 1-5 μg/mL recombinant flaA antigen in carbonate-bicarbonate buffer (Sigma) to each well and incubating overnight at 4° C. Plates were washed 3 times with 300 μL PBS supplemented with 0.05% Tween-20 (PBS-T). Washed plates were blocked with 250 μL PBS-TM supplemented with 5% non-fat dry milk (PBS-TM) for 2 hours at room temperature. Blocking solution was discarded and 100 μL of sample containing VHH was added to each well. VHH samples were prepared by diluting purified protein or spirulina extracts with PBS-TM, and samples in a dilution series were serially diluted with PBS-TM. Samples were incubated at room temperature for 1 h to allow VHH binding to antigen. After incubation, plates were washed with 300 μL PBS-T 3 times. Wash was discarded, 100 μL of primary antibody diluted with PBS-TM was added to each well, and plates were incubated at room temperature for 1 h. A 1:10,000 dilution of either a mouse anti-His-tag antibody (GenScript) or rabbit anti-camelid VHH antibody cocktail (GenScript) was used as the primary antibody. After incubation, plates were washed 3 times with 300 μL PBS-T, and 100 μL of a secondary antibody was added to each well. An HRP-conjugated goat anti-mouse antibody or HRP-conjugated donkey anti-rabbit antibody was used as the secondary antibody. Plates were incubated at room temperature for 30-45 min at room temperature. Plates were washed twice with PBS-T and once with PBS. Plates were developed using either a SeraCare KPL TMB Microwell Peroxidase Substrate System (Sera Care Life Sciences) or 1-Step Ultra TMB-ELISA Substrate Solution (Thermo Scientific) following the manufacturer's recommendation. Peroxidase activity was quenched after 5-10 min with 50 μL of 1 M HCl or 2 M sulfuric acid. Absorbance at 450 nm was measured on an M2 plate reader (Molecular Devices). Data analysis was performed using Prism (GraphPad Software).

### Kinetics Binding Analysis of VHHs

Kinetics binding measurements for were performed by biolayer interferometry (BLI) using an Octet Red96e (Forte Bio). Biotinylated MBP-flaA was loaded onto streptavidin biosensors with a loading concentration of 100 nM and loading time of 4 min. After loading, probes were allowed to reach a baseline equilibrium in kinetics buffer (PBS with 1% bovine serum albumin and 0.05% Tween-20) for 2 min. Association and dissociation were monitored for 20 s and 140 s respectively. Purified aa682 diluted with kinetics buffer was assayed at concentrations from 1 μM to 10 nM. The 10 nM sample was excluded from analysis for weak signal. Two biosensors were used as references: a 0 nM aa682 control, as well as a no ligand control. Kinetics binding values were determined using Octet Data Analysis HT software (ForteBio). Curve fits were performed using a global fit across all concentrations of aa682 and assuming a 1:1 binding model.

### Epitope Mapping of VHH/Antigen Interaction

The epitope mapping of the interaction between FlagV6 and flagellin was performed using phage displayed peptide fragments derived from a ˜300 amino acid soluble fragment of C. jejuni flaA. A sliding window of 30 amino acid fragments, with a 2 amino acid interval along the length of flaA, was designed as oligos for cloning into the phagemid pADL-23c (Antibody Design Labs). The peptide library was cloned into the BglI site of the phagemid by Gibson Assembly and transformed into DH5a E. coli, yielding>6×10⁴ transformants. The phagemid library was cleaned up by QiaPrep Spin Minikit columns and transformed into electrocompetent TG1 cells (Lucigen). Phage production was induced with the pIII deficient helper phage CM13d3 (Antibody Design Labs) to ensure polyvalent display of the peptide epitopes. Phage from an overnight culture in 2xYT media was precipitated and washed following the manufacturer's protocol. Wells of an ELISA plate were coated overnight with 100 μL of 1 μg/mL FlagV6 VHH in carbonate-bicarbonate buffer, washed with PBS-T, and blocked with PBS-TM. The phage library was diluted with PBS-TM to a concentration of 1012 phage/mL and incubated at room temperature for 30 min. The phage were then panned for VHH binders by adding 100 μL of blocked phage to wells of the ELISA plate and incubating on a vibrating platform for 2 h at room temperature. Unbound phage were washed from wells with 6, 300 μL washed with PBS-T. Bound phage were eluted with low pH by adding 100 μL of 100 mM glycine, pH 2.0, incubating for 5 min with shaking. The elution buffer was neutralized with 40 μL of 2 M Tris, pH 7.5 and used to reinfect phage competent TG1 cells (Antibody Design Labs). The library amplification and panning were performed for 2 additional rounds. After the third round of panning, all phagemid-containing colonies were observed to contain the same peptide fragment by Sanger sequencing. Two independent replicates of the experiment yielded overlapping fragments that mapped to the D3 domain of flaA.

Flow Cytometry of VHH Binding to C. jejuni

The binding of spirulina expressed VHHs to a pure culture of C. jejuni was measured by flow cytometry. An aliquot of lysate prepared from spray dried spirulina biomass was incubated with an equivalent volume of 107 cfu/mL of C. jejuni 81-176 for 1 h at 4° C. After washing with PBS containing 2% FBSs, bacteria were incubated for 30 min with the anti-His-tag antibody [iFluor647] (GenScript). Samples were washed with PBS containing 2% FBS, resuspended in 2% paraformaldehyde and acquired on LSR Fortessa flow cytometer (BD Biosciences) using FCS and SSC parameters in logarithmic mode. Data were analyzed using the FlowJo software (TreeStar) or FACS Diva software (BD Biosciences).

### Motility Inhibition Assay

The motility-inhibiting activity spirulina-expressed aa682 was measured by the motility of C. jejuni through soft agar. All C. jejuni culturing was performed in a tri-gas incubator at 40° C. under microaerobic conditions (5% 02, 10% CO₂) unless otherwise stated. Glycerol stocks of C. jejuni were first streaked on Campy Blood Agar Blaser plates (Thermo Scientific) and grown for 48 h. Bacteria were then used to inoculate 0.4% soft agar Mueller-Hinton plates by stab and incubated for 48 h. A slice of agar from the leading edge of the motility halos was used to inoculate 10 mL of Mueller-Hinton broth. Liquid cultures were incubated under standard conditions for 72 h. A spot of 20 μL of 5 mg/mL of purified aa682 in PBS was added to the center of soft agar Mueller-Hinton plates and allowed to fully adsorb into the agar. VHH spots were inoculated with 1 μL of OD₆₀₀=0.03 of C. jejuni from the liquid culture. A samples and controls were set up in triplicate. Plates were incubated under standard conditions. The diameter of the motility halos was periodically measured and used to calculate area.

### Mid-Scale Production of Spirulina Biomass for Pre-Clinical Trials

To prepare biomass for pre-clinical mouse trials, the scale of spirulina culturing was increased, and harvested biomass was spray dried. Spirulina cultures were initially propagated in shake flasks in media based on the standard cyanobacterial SOT media in Multitron conditions. Shake flask cultures were used to inoculate airlift reactors, with media modified by partial replacement of sodium bicarbonate with sodium carbonate, such that initial culture pH was 9.8. Cells were grown at light levels between 500 and 2500 μmol/m2/sec, with temperature maintained at 35° C. As the culture utilizes CO₂ and grows the pH rises, and CO₂ is added to the airlift stream to maintain pH between 9.8-10. Cultures were inoculated at a concentration of 0.1-0.5 g biomass per liter by ash-free dry weight, and harvested by filtration at 2-4 g/L. To prepare for spray drying, the harvested biomass was rinsed with a dilute 0.1% trehalose solution (to remove excess media salts), concentrated again by filtration, and then spray dried in a centrifugal nozzle spray dryer. Feed rate, airflow, and inlet air temperature were controlled to maintain an outlet air temperature of 68-72° C. at the powder separation hydrocyclone. Once collected from the hydrocyclone, the powder was sealed and stored in airtight, opaque mylar bags to prevent exposure to moisture or light. The powder is stored at room temperature. Prior to use in animal trials, spirulina biomass was analyzed to confirm strain identity. Dried biomass was genotyped to confirm the presence of the correct transgene and the absence of contaminating sequences (see above). CEIA and ELISA binding assays (see above) were also performed to confirm expression and binding activity of the spirulina-expressed VHH.

### Example 4—Expression of Camelid Single-Chain Antibody Fragments in Spirulina

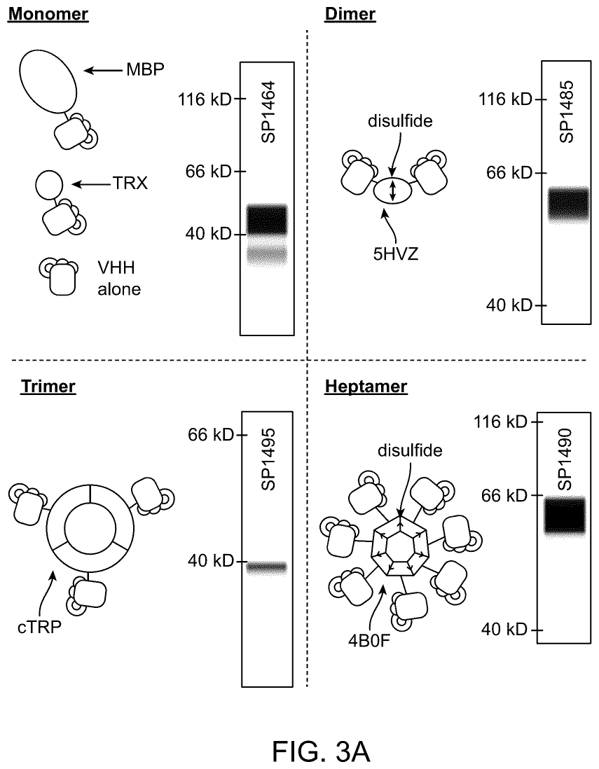

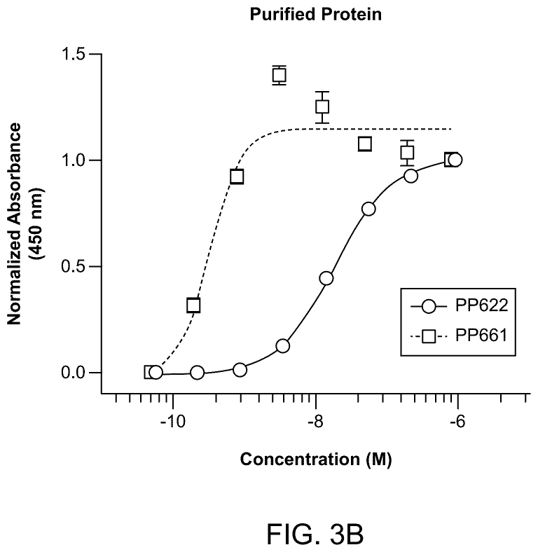

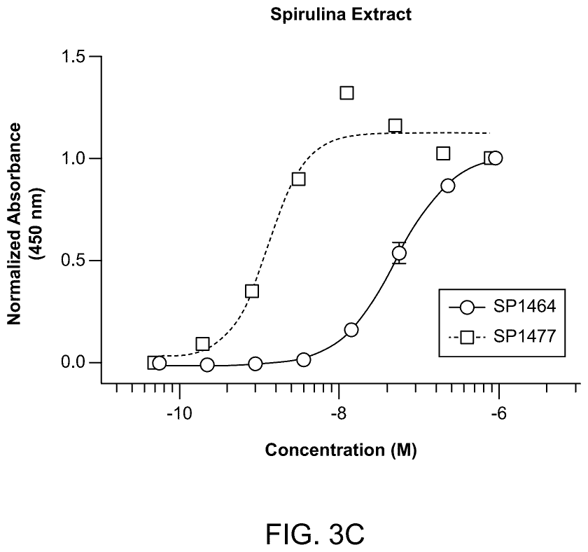

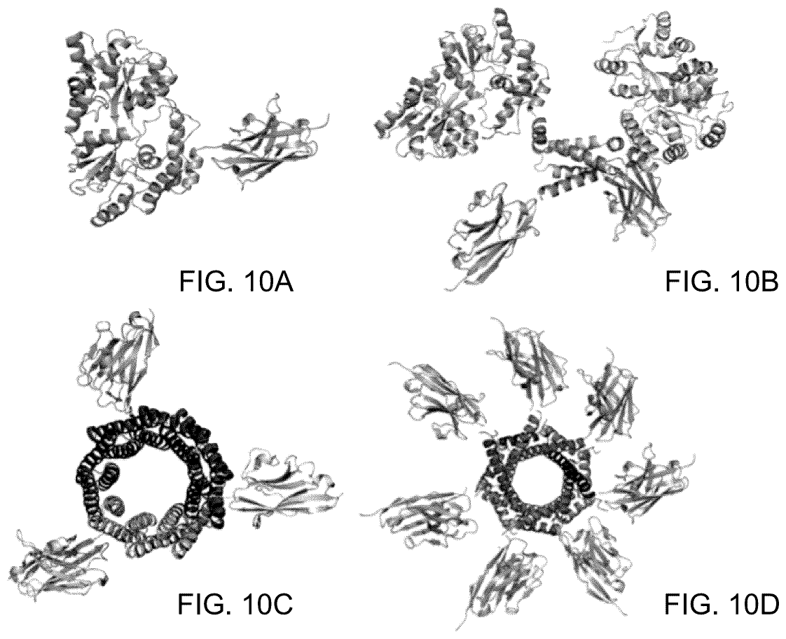

Unlike human antibodies, antigen-binding domains (VHHs) derived from camelid single-chain antibodies are ideal for expression in prokaryotes, like spirulina, because neither intracellular formation of disulfide bonds nor specific glycosylation is required for synthesis of the bioactive protein. VHHs were constitutively expressed in spirulina from the strong promoter P_{cpc600} in various formats, including monomers, dimers, trimers and heptamers, see FIG. 10A-FIG. 10D. Monomeric VHHs were typically expressed as a fusion protein with a solubility enhancing chaperone, such as the E. coli maltose-binding protein (MBP). Multimers were constructed using scaffolding strategies, and routinely demonstrated sub-nanomolar K_{d} values, see FIGS. 3A-3C. Expression levels varied among VHHs, ranging from 0.5% to 20% of soluble protein. Because they are easily expressed in prokaryotes, VHHs can be rapidly isolated from high diversity, naive phage-display libraries. These typically have mid-nM affinities for their antigen targets, and therefore further mutagenesis may be required to achieve the higher affinities required for therapeutic efficacy. Expression of VHHs as high-avidity multimers, as described here, bypasses this requirement and therefore can accelerate product development.

### Example 5—Spirulina Strains Expressing an Anti-Campylobacter VHH

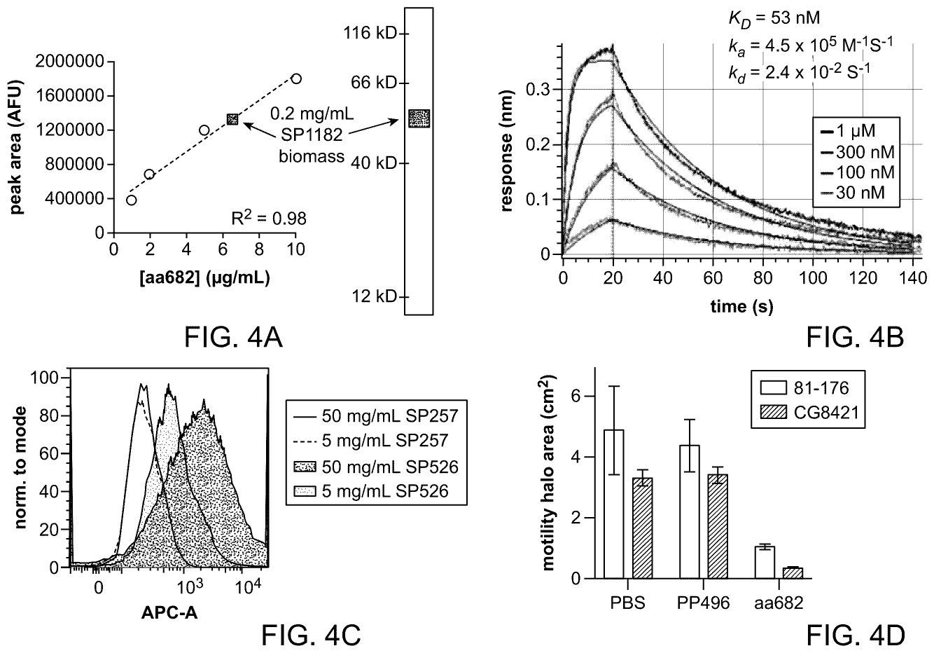

The VHH FlagV6 binds the flagellin (FlaA), a subunit of C. jejuni flagella with a KD of 25 nM34. The binding site was mapped to the D3 domain of FlaA by phage display of peptides tiled across the entire FlaA protein (FIG. 4A). Spirulina strain SP526 used P_{cpc600} to drive expression of a monovalent fusion polypeptide of FlagV6 VHH and MBP. Two alterations were introduced into the N-terminal portion of the framework region of FlagV6 to confer increased resistance to chymotrypsin (K3Q and E5V). This anti-campylobacter protein was designated aa682. (SP1182 a markerless version of SP526 was subsequently constructed for clinical testing.) Following segregation to homozygosity, single filaments were isolated, and an axenic strain was derived. Constitutively expressed aa682 protein amounts were determined by capillary electrophoresis immunoassay (CEIA) to be approximately 3% of biomass (FIG. 4A). Similarly, to the reported affinity of FlagV6, aa682 was found to bind to a recombinant FlaA construct with a KD of 53 nM (FIG. 4B). The VHH-binding site was mapped to the D3 domain of FlaA by phage display of peptides tiled across the surface-accessible region of FlaA. The D3 domain of FlaA protrudes from the axis of the flagellum, and should be surface accessible for VHH binding. Specific binding of aa682 to intact C. jejuni flagella was demonstrated by flow cytometry. Aqueous extracts of a spirulina strain (SP526) expressing an analog of aa682 were incubated with a pure culture of C. jejuni 81-176 (107 colony forming units (CFU)/mL) and then stained with anti-His-tag antibody to detect the binding of the VHHs to the pathogen. Binding to C. jejuni was compared to an extract from a spirulina strain (SP257) that expressed an irrelevant VHH (FIG. 4C). The major flagellin protein FlaA is required for motility, and motility is required for virulence in vivo. Binding of VHHs to FlaA has previously been shown to prevent campylobacter motility in vitro. Purified aa682 blocked campylobacter motility on agar plates of 2 different strains of C. jejuni (FIG. 4D) and was therefore predicted to inhibit campylobacter pathogenesis in vivo.

### Example 6—Preventing Campylobacter Disease In Vivo

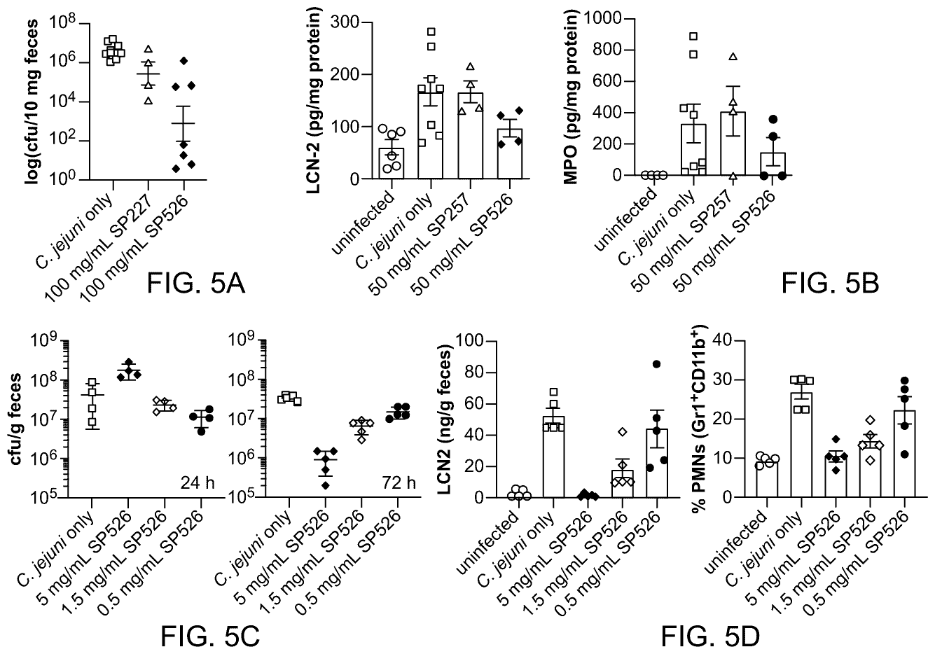

Two independent mouse models were used to test whether orally delivered spirulina biomass containing an anti-campylobacter VHH could prevent enteric campylobacter infection. In the first, mice were rendered susceptible to campylobacter infection by an antibiotic pretreatment regimen, and then challenged on day 0 with 10⁶ CFU of C. jejuni 81-176 by oral gavage. Spirulina biomass used for treatment and control groups was cultured in mid-scale bioreactors, washed, and spray dried. Untreated controls, or controls treated with either spirulina biomass containing no recombinant protein (SP227; engineered for phycocyanin production), or spirulina biomass containing an irrelevant VHH, were compared to mice given SP526 by oral gavage prior to campylobacter challenge, and again on days 1 and 2. Treatment with SP526 reduced campylobacter fecal shedding by 3 to 4 orders of magnitude, and significantly reduced two biomarkers of intestinal inflammation, lipocalin-2 (LCN-2) and myeloperoxidase (MPO) (FIG. 5A, 5B). It was observed that all of the mice in the control groups, but none of the mice treated with SP526, suffered from diarrhea following campylobacter infection. The severity of this overt clinical disease was not quantified. A second model also used an antibiotic preconditioning regimen but a challenge dose of 10⁸ CFU of C. jejuni 81-176. Dose-ranging experiments showed that a single prophylactic dose of 2 mg of dry SP526 biomass administered by oral gavage was sufficient to prevent campylobacteria disease, as measured by stool lipocalin-2 and myeloid cell infiltration (PMNs) of the cecum. Furthermore, this single dose accelerated campylobacter expulsion from the gut in the first 24 hours after challenge, which was accompanied by reduced campylobacter shedding at 72 hours after challenge (FIG. 5C, 5D).

### Example 7—Large-Scale Continuous Growth

Open pond systems are used to cultivate spirulina at commercial scale for production of food, feed, and pigments, but uncontrolled exposure to environmental contaminants make these challenging for the manufacture of biopharmaceuticals under FDA cGMP. Therefore, an indoor, pH-controlled, air-mixed photobioreactor platform was built around a modular 160 L-2,000 L vertical flat-panel reactor that is scalable to commercial size suitable for the manufacturing of biopharmaceuticals. Advantages of this platform are the exceptionally low cost of large-scale growth and downstream processing. A factor contributing to the low upstream production cost was that spirulina thrive under extreme conditions (pH>10 and high total salinity) that severely limit the growth of adventitious organisms. In addition, spirulina, being photoautotrophic, have simple nutritional requirements; no carbon-based source of energy (i.e., sugar feedstock) is required. Together these features allowed the use of unsealed reactors under sanitary, but not aseptic, conditions. Total microbial counts in the final product were within specified limits, and absence of pathogenic bacterial confirmed regularly. Utilizing sodium nitrate allowed for high nitrogen levels in the formulated media without the toxicity that could result from the use of ammonia, and a high initial nitrogen level obviated the need for monitoring or re-feeding of nitrogen during growth. Single-use polyethylene bags contained the spirulina culture, further reducing what is typically one of the biggest cost components in any biopharmaceutical process: sterilization downtime.

In brief, spirulina cultures were grown at large scale (250 L) in airlift reactors following protocols similar to the mid-scale reactors described above. Cultures were inoculated into the same media described above for mid-scale cultures at a concentration of 0.1-0.5 g biomass per liter by ash-free dry weight, grown under identical temperature and pH controls, and harvested by filtration over stainless steel screens at 2-4 g/L. A portion of the harvested culture was used to inoculate serial cultures, and the remaining harvested biomass was used for spray drying as above. The dried powder was sealed and stored at room temperature in airtight, opaque mylar bags to prevent exposure to moisture or light. Post collection, quality control of powder lots include determination of concentration of the 6×-his tagged protein using CEIA conducted on a Jesssystem (ProteinSimple). Specific ligand binding activity is determined on an Octet Red96e biolayer interferometry instrument (Forte Bio) using recombinant, biotinylated C. jejuni flaA protein attached to streptavidin coated biosensors. In addition, microbial characterization by USP and, and elemental impurities determined by USP.

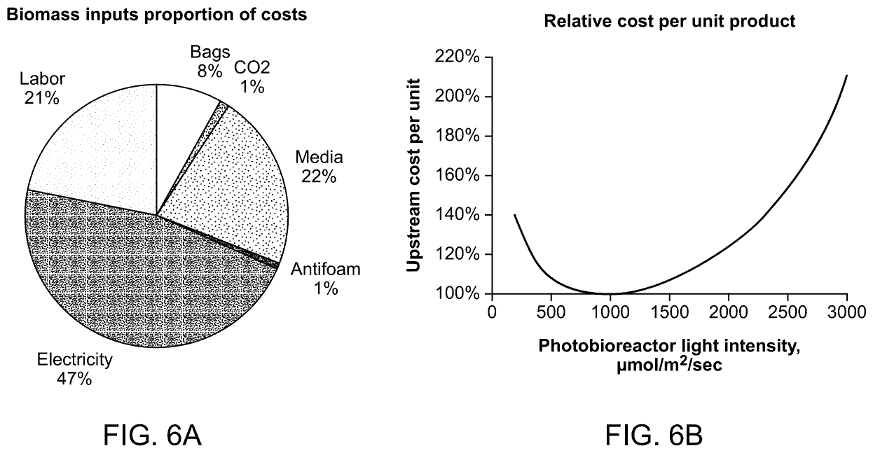

The energy cost for LED illumination was the major component of production cost (FIG. 6A). The reactors were illuminated from both sides of the culture using off-the shelf commercial full spectrum LEDs with adjustable intensity. The complex relationship between the capital and operational costs of biomass growth were evaluated as a function of light intensity, and an optimum that achieved the greatest productivity per unit energy cost was identified (FIG. 6B). Cultures were continuously maintained for sequential 1-week growth cycles. On a weekly basis, cell densities reached approximately 4 g/L, at which point the biomass was harvested by pumping over a series of stainless-steel screens to concentrate and rinse the slurry. A portion of the slurry was used to re-inoculate the reactors, and the remainder was processed into drug product.

### Downstream Processing

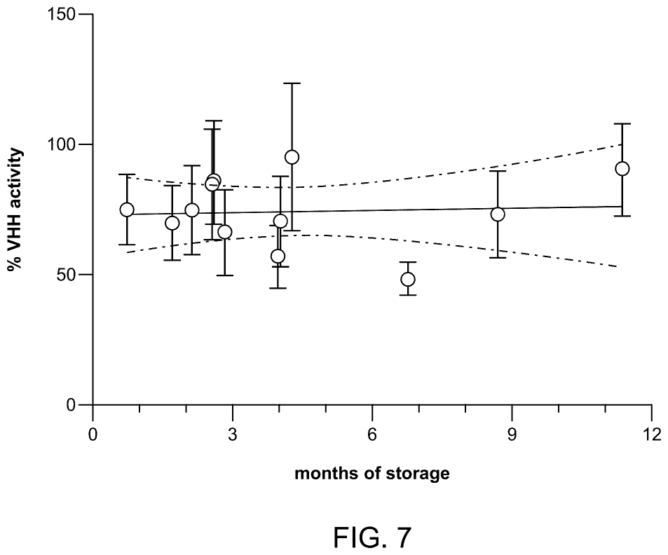

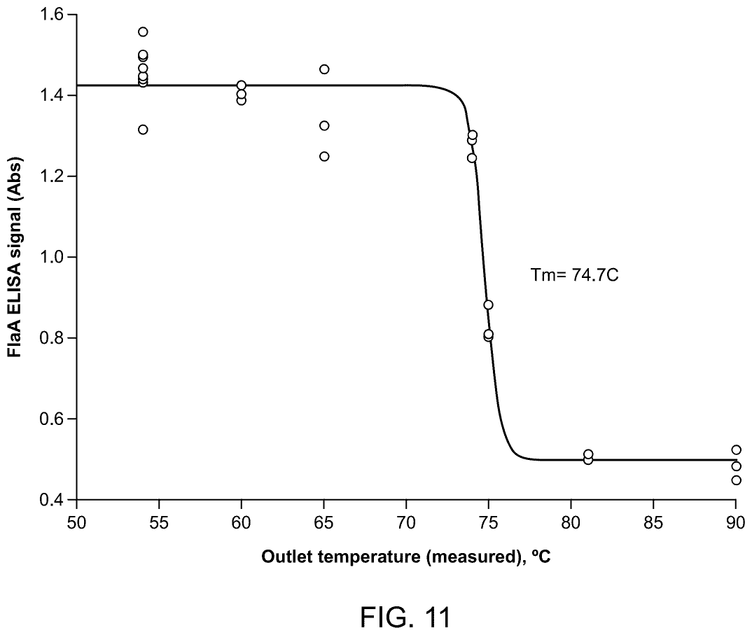

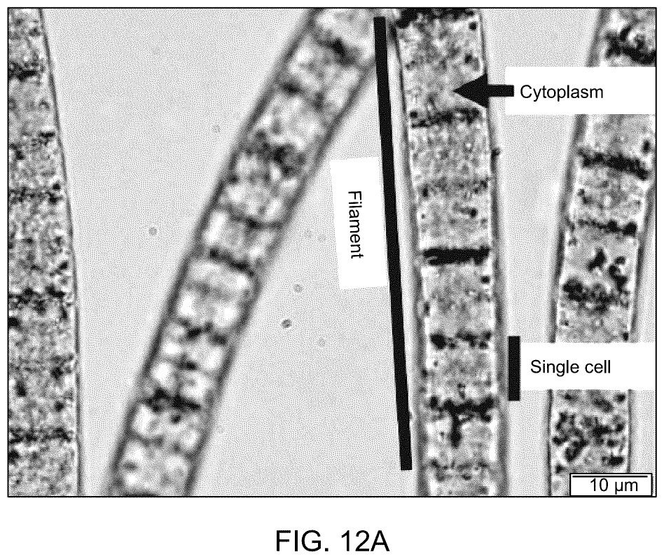

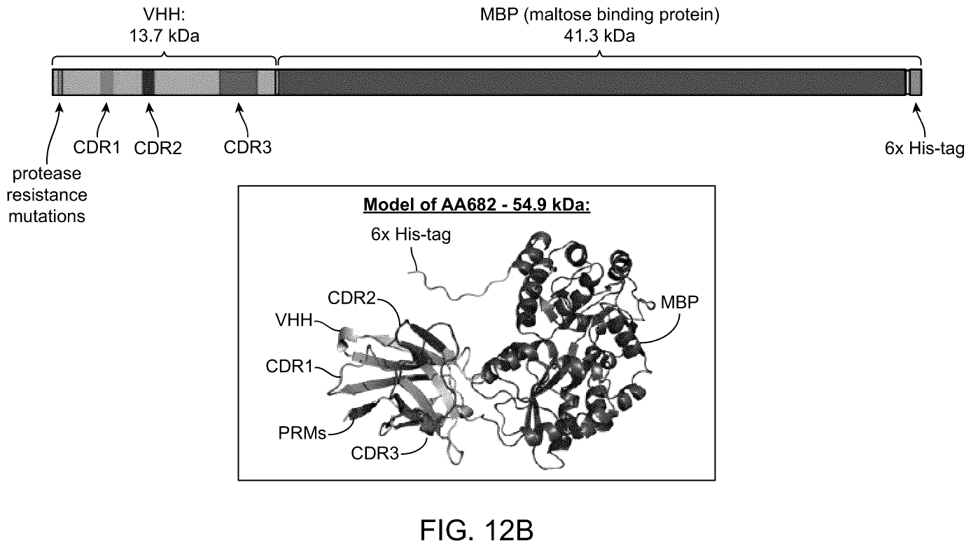

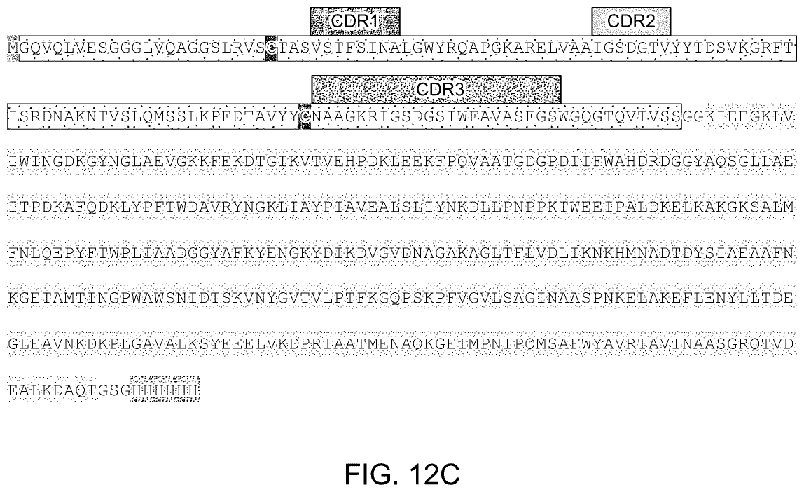

The concentrated spirulina slurry was rinsed with a dilute trehalose solution, and then spray dried. A large parameter space was evaluated at laboratory scale for efficiency of drying, moisture content, and retention of antibody activity. Conditions were identified, see FIG. 11, in which suitable system efficiency was achieved while maintaining greater than 90% of antibody activity. The process was translated to a larger scale (5 kg/hr) spray dryer equipped with a centrifugal atomization system. With only minor optimization of system throughput, inlet and outlet temperatures and air flow rates, equivalent dryer performance at a about 20× single step scale-up was achieved. Once dried, the powder was collected and sealed in light- and moisture-proof packaging; antibody activity was retained at room temperature for at least 1 year (FIG. 7). Packaging the powder into standard vegetarian capsules was the final downstream process. Because of product stability, distribution can be cold-chain independent. The system was third party audited and found compliant with ISO 22000 Food Safety Management System standards and subsequently required minimal upgrades to satisfy pharmaceutical cGMP requirements

### Long-Term Stability of Dried Spirulina Biomass

Batches of SP1182 spray dried biomass were stored at room temperature and collectively assessed for binding activity by ELISA. Duplicate biomass samples from each batch were resuspended in PBS and lysed by freeze-thaw extraction and clarified by centrifugation. The binding activity of aa682 present in the lysates was determined by ELISA with a recombinant flaA antigen as described above. Purified aa682 was used to generate a standard curve for binding activity by linear regression. The standard curve was used to calculate the concentration of aa682 in the SP1182 lysates. The percentage of expected VHH activity was determined by normalizing the aa682 concentration in each lysate to an assumed concentration of 3% aa682 per unit biomass.

### In Vitro Gastric Digests of Dried Spirulina Biomass

Spray dried SP1182 biomass was exposed to simulated gastric fluid (SGF) to determine the stability of aa682 present in spray-dried spirulina. A sample of spray-dried SP1182 biomass was resuspended in PBS at 30 mg/mL. This resuspension was diluted 1:30 with pre-chilled SGF (50 mM citrate-phosphate buffer pH 3.0, 94 mM NaCl, 13 mM KCl, pH 3.5 with 2000 U/mL pepsin (MP Biomedicals)) and incubated in a 37° C. water bath. Protease activity was neutralized by adding 50 mM NaOH and 1 mM phenylmethylsufonyl fluoride (PMSF). Samples were pelleted by centrifugation at 14,000 RPM for 5 min. Biomass pellets were solubilized using 1× NuPAGE LDS sample buffer to a final biomass concentration of 1 mg/mL and heated at 90° C. on heat block for 10 min. A similar process was used to assess the stability of purified aa682, absent the centrifugation step. The stability and activity of biomass-encapsulated aa682 after exposure to low pH, simulated gastric buffer was assessed by CEIA and ELISA binding assay. Spray-dried SP1182 biomass was resuspended in either 50 mM bicarbonate buffer or citrate-phosphate buffer, pH 3.0 with 1 mM PMSF. Samples were incubated in a 37° C. water bath for 60 min. After incubation, biomass resuspensions were pelleted at 10,000 RPM for 5 min. The supernatant was transferred into fresh tubes and stored at 4° C. Pellets were resuspended in 1 mL of 50 mM bicarbonate buffer to a final biomass concentration of 30 mg/mL and incubated in 37° C. water bath for 30 min. Resuspensions were treated to 3 cycles of flash freezing in liquid nitrogen followed by thawing at 37° C. to extract soluble protein. After the last thawing, samples were pelleted using a refrigerated tabletop centrifuge for 30 min at maximum speed to separate soluble protein from insoluble cellular debris. The supernatant was used to measure the expression level and binding activity of aa682 by CEIA and ELISA respectively.

In Vitro Protease Digests with Intestinal Proteases

To measure intestinal protease resistance, SP1182 lysates were digested with trypsin and chymotrypsin and VHH binding activity was assessed by ELISA. Total soluble extract was prepared from a resuspension containing 40 mg of dried SP1182 biomass per mL of Bis-Tris buffer (20 mM Bis-Tris, pH 6.0) by the freeze-thaw protocol described above. Two volumes of soluble extract were mixed with 1 volume of protease in bis-tris buffer and 1 volume of PBS to yield a final digest concentration of 0.1 or 0.01 mg/mL of trypsin or chymotrypsin with a reaction pH of ˜6.5. Digests were performed at 37° C. for 1 h with shaking at 900 rpm on an Eppendorf Thermomixer. Protease activity was neutralized by adding an equivalent volume of 2 mM PMSF and 2× Pierce Protease Inhibitor Mini tablets (Thermo Scientific) in PBS. Binding activity of VHH to recombinant flaA was measured by ELISA as described above.

### Example 8—Evaluation of Targeted Delivery to the Gastrointestinal Tract

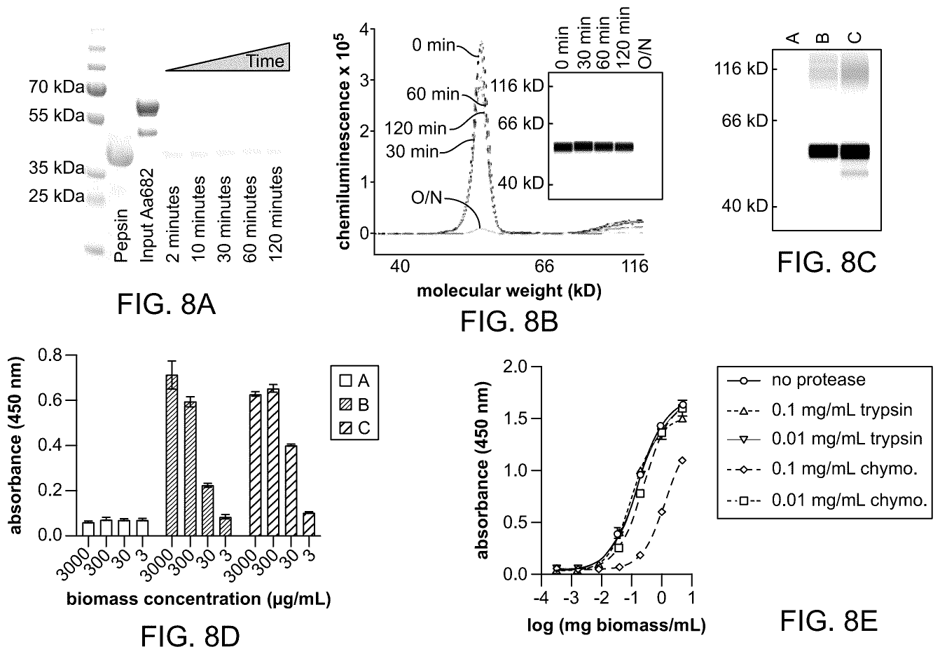

A challenge associated with direct delivery of protein therapeutics to the gastrointestinal tract is protease digestion. Upon ingestion, biologics are initially subjected to the low pH and high pepsin gastric environment, so it was evaluated whether bioencapsulation of therapeutic proteins within dry spirulina biomass would provide protection. Purified aa682 was fully degraded within 2 minutes of incubation in simulated gastric conditions (FIG. 8A). However, when delivered within dry spirulina biomass more than 70% of aa682 remained intact after 2 hours of incubation in the same gastric conditions (FIG. 8B). Transition of biomass to the higher pH of a simulated duodenal environment was then sufficient to extract more than 90% of the encapsulated aa682 from spirulina within 60 minutes (FIG. 8C). When assayed by ELISA, the binding activity of the extracted aa682 was unaffected by the initial biomass incubation in the simulated gastric environment (FIG. 8D). In vivo efficacy is also dependent upon the sensitivities of therapeutic proteins to proteases of the small intestine, especially trypsin and chymotrypsin. Empirical analysis is necessary to identify VHHs with appropriate metabolism-resistant properties, which range from highly sensitive to almost complete resistance to intestinal proteases. When soluble extract from SP1182 was treated with proteases, aa682 was resistant to the constitutive intestinal levels of both trypsin and chymotrypsin. Some sensitivity of aa682 to chymotrypsin, but not trypsin, was evident when the SP1182 extract was exposed to the high induced protease levels that are present immediately after ingestion of a meal (FIG. 8E).

### Example 9—Prophylactic Treatment of C. jejuni Infection in 2 Mouse Models

Two independent mouse models were used to test the efficacy of spirulina-expressed VHHs in treating C. jejuni infection. In a pilot experiment with the first model of C. jejuni infection, mice were fed a zinc-deficient diet (dZD)38 prior to challenge. Animals were maintained per institutional protocols and fed a regular diet with ad libitum water for 3 days. Animals were then started on the study diet for 10 days, after which water was replaced with water containing an antibiotic cocktail for 3 days to condition gut flora for C. jejuni colonization. Water was replaced with untreated, antibiotic-free water for 1 day prior to C. jejuni challenge. On day 0, mice were given an inoculum of 106 live C. jejuni cells (resuspended in PBS), strain 81-176, by oral gavage. Food and water were provided ad libitum throughout. Mice were given 5 doses of a spirulina resuspension before and after challenge. Spray dried spirulina biomass was resuspended in PBS at a concentration of 10% (w/v). A 200 μL resuspension was delivered by oral gavage on days −1, 0, 1, 2, and 3, relative to challenge. Day-of-challenge dosing was administered 60 min. prior to challenge. Food and water were withdrawn 30 min. prior to treatment, then provided ad libitum. To assess efficacy, mice were monitored for symptoms of diarrhea, changes in weight, and bacterial shedding in stool. Weight measurements were made daily for 7 days. Stool samples were collected on days 1, 3, and 7 post-challenge. A second experiment using the first model of infection involved a change of study diet and a reduced spirulina dose. Animals were fed a Regional Basic Diet (RBD) for 10 days, followed by 3 days of antibiotic treatment. Untreated water was provided for 1 day prior to C. jejuni challenge. On day 0, mice were given an inoculum of 106 live C. jejuni cells (resuspended in PBS), strain 81-176, by oral gavage. Food and water were provided ad libitum throughout. Mice were given 3 doses of spirulina before and after challenge. On days −1, 0, and 1 relative to challenge, mice were orally gavaged with 200 μL of spirulina resuspension or control. Day-of-challenge dosing was administered 60 min prior to challenge. Food and water were withdrawn 30 min prior to treatment, then provided ad libitum. Spirulina resuspension was prepared at a concentration of 2% (w/v) in PBS. To assess efficacy, mice were monitored for changes in weight, bacterial shedding in stool, and levels of biomarkers in cecum. Weight measurements were made daily for 7 days. Stool samples were collected on days 2, 4, 6, 8, and 10 post-challenge. On day 11, the levels of lipocalin-2 (LCN-2) and myeloperoxidase (MPO) were measured in stool and cecal contents by ELISA (DuoSet ELISA Mouse Lipocalin-2/NGAL, R&D Systems).

In the second model of C. jejuni infection, mice were orally treated with a range of spirulina concentrations to identify the minimally effective prophylactic dose of therapeutic. Mice were housed, 5 per cage, under standardized conditions (20±2° C., 55±8% relative humidity, 12 h light/dark cycle). Food and water were available ad libitum, and mice were monitored daily. Mice were pre-treated orally with 10 mg of vancomycin in 200 μL PBS at 48, 24 and 12 h prior to spirulina administration. A single 400 μL dose of spray dried spirulina resuspended in PBS was administered by oral gavage to mice 1.5 h before infection with C. jejuni 81-176 (108 cfu/200 μL PBS). To monitor the efficacy, mice were observed daily, and stools were collected at 24, 48 and 72 h post infection. To monitor the pathogen load, stools were resuspended and plated on Mueller Hinton agar plates containing 10 μg/mL of vancomycin and trimethoprim. The cecal polymorphonuclear neutrophils (PMNs) were measured by flow cytometry 72 h post infection. Mice were sacrificed, the caecum was removed, opened longitudinally, delicately separated by caecal content and washed twice with ice cold PBS. The caecum was digested twice with RPMI and EDTA 5 mM for 30 min at 37° C. The filtrated fragments were then digested in RPMI 5% FBS (fetal bovine serum), 1 mg/mL collagenase type II, 40 μg/mL DNase-I for 40 min. The filtered suspension, containing the caecum lamina propria cells, was centrifuged for 5 min at 1,500 rpm and resuspended in RPMI complete medium. Single-cell suspensions from caecal lamina propria were stained with labelled antibodies diluted in PBS with 2% FBS for 20 min on ice. The following mouse antibodies (mAbs) were used: APC conjugated anti-CD11b (1:200), PE conjugated anti-GR1 (1:200). Samples were acquired on an LSR Fortessa (BD Biosciences) flow cytometer. Data were analysed using the FlowJo software (TreeStar, Ashland, OR, USA) or FACS Diva software (BD Biosciences, Franklin Lakes NJ, USA). The inflammation status of mice was evaluated by measuring faecal lipocalin-2 (LCN-2) levelsin fecalsupernatants by ELISA (DuoSet ELISA Mouse Lipocalin-2/NGAL, R&D Systems). Briefly, feces collected at sacrifice were resuspended at 0.01 g per 100 μL PBS, centrifuged for 10 min at maximum speed, and diluted before performing the ELISA per the manufacturer's instructions.

### Example 10—Phase I First-In-Human Clinical Trial

Spirulina strain SP1182 was cultured in large-scale bioreactors under cGMP conditions and used to formulate the drug product LMN-101, a 55 kDa fusion protein (C₂₄₆₇H₃₈₀₆N₆₅₀O₇₄₅S₉) consisting of a camelid antibody VHH domain (13.7 kDa) that binds the flagellin protein flaA from C. jejuni, a short linker, maltose binding protein (41.3 kDa) and a c-terminal 6× histidine tag.

To assess the safety and tolerability of LMN-101, a Phase 1 clinical trial was designed and conducted. Eligible, healthy volunteers aged 18-50 were enrolled following informed consent. The study was performed in accordance with ICH guidelines and in compliance with all applicable laws and regulations. A total of 20 subjects were randomized to active or placebo treatment as described in the following sequence

Clinical Trial Randomization Scheme
3000 mg of LMN-101, administered orally as 6 500-mg capsules as a single dose (2 subjects).
300 mg of LMN-101, administered orally as a single 300-mg capsule 3 times daily for 28 days (4 subjects) or identical-appearing placebo capsule (2 subjects).
1000 mg of LMN-101, administered orally as 2 500-mg capsules 3 times daily for 28 days (4 subjects) or identical-appearing placebo capsules (2 subjects).
3000 mg of LMN-101, administered orally as 6 500-mg capsules 3 times daily for 28 days
   (4 subjects) or identical-appearing placebo capsules (2 subjects). 1 additional subject in this cohort who dropped off study was replaced.

This Phase 1 trial demonstrated LMN-101 was safe and well tolerated at the doses tested. There were no statistically significant differences between the active and placebo groups. There were no significant adverse events or significant laboratory abnormalities reported during or following the trial. Rates of adverse events, deemed possibly related, were similar between the two groups. Reported adverse events were mild and consisted of nausea, abdominal pain, diarrhea, gastroesophageal reflux, constipation, pharyngitis, and delayed menstruation. Laboratory evaluation demonstrated there was no significant VHH absorption.

### INCORPORATION BY REFERENCE

This patent application incorporates by reference in their entireties for all purposes the following patent publications and applications: U.S. 63/140,577, U.S. Pat. No. 10,131,870, WO 2019/222711 filed May 17, 2019, and PCT/U S2020/040794 filed Jul. 2, 2020.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### REFERENCES

1. Vonshak, A. Spirulina Platensis Arthrospira. (CRC Press, 2002).
2. Gershwin, M. E. & Belay, A. Spirulina in Human Nutrition and Health. (CRC Press, 2007).
3. Cohen, S. N., Chang, A. C., Boyer, H. W. & Helling, R. B. Construction of biologically functional bacterial plasmids in vitro. Proc. Natl. Acad. Sci. U.S.A. 70, 3240-3244 (1973).
4. Regist, F. A. D. A. F. 2002. Guidance for industry drugs, biologics and medical devices derived from bioengineered plants for use in humans and animals.
Jeamton, W., Dulsawat, S., Tanticharoen, M., Vonshak, A. & Cheevadhanarak, S. Overcoming Intrinsic Restriction Enzyme Barriers Enhances Transformation Efficiency in Arthrospira platensis C1. Plant and Cell Physiology 58, 822-830 (2017).
6. Klanchui, A. et al. SYSTEMS BIOLOGY AND METABOLIC ENGINEERING OF ARTHROSPIRA CELL FACTORIES. Computational and Structural Biotechnology Journal 3, e201210015-8 (2012).
7 Fang, M. et al. Rapid mutation of Spirulina platensis by a new mutagenesis system of atmospheric and room temperature plasmas (ARTP) and generation of a mutant library with diverse phenotypes. PLoS ONE 8, e77046 (2013).
8. Taton, A. et al. The circadian clock and darkness control natural competence in cyanobacteria. Nature 35, 43-37 (2020).
9. Mell, J. C. & Redfield, R. J. Natural Competence and the Evolution of DNA Uptake Specificity. Journal of Bacteriology 196, 1471-1483 (2014).
10. Kawata et al. Transformation of Spirulina platensis strain C1 (Arthrospira sp. PCC9438) with Tn5 transposase-transposon DNA-cation liposome complex. Marine Biotechnology. 6, 355-363 (2004).
11. Toyomizu, M. et al. Effective transformation of the cyanobacterium Spirulina platensis using electroporation. J Applied Phycology. 13, 209-214 (2001)
12. Yuanyuan, C. Breeding and characterization of amino acid-analogue-resistant mutants of Arthrospira platensis. World Journal of Microbiology and Biotechnology, Rapid Communications of Oxford. 30, 423-428 (2013).
13. Zhang, et al., Preliminary Studies on the Genetic Transformation of Spirulina Platensis, Algae and their Biotechnological Potential, Proceedings of the 4th Asia-Pacific Conference on Algal Biotechnology, July 3-6,2000, in Hong Kong, Springer, NL, Jan. 1, 2011, pp. 263-269

1. A method of transforming a population of spirulina cells comprising growing the spirulina cells with:
(a) a co-culturing microorganism to induce competence; and
(b) a transforming molecule.

2. The method of claim 1, wherein the co-culturing microorganism is gram-negative.

3. The method of claim 1, wherein the co-culturing microorganism is gram-positive.

4. The method of claim 1, wherein the co-culturing microorganism is aerobic.

5. The method of claim 1, wherein the co-culturing microorganism belongs to the genus Sphingomonas.

6. The method of claim 1, wherein the co-culturing microorganism is selected from: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Akanivorax, Dyella, Rhodanobacter, Halomonas, Variovorax, Frateuria, Dokdonella, Cupriavidus and Oscillatoria, Koinonema, Oxynema, Planktothrix, and Microcystis.

7. The method of claim 1, wherein the co-culturing microorganism belongs to the genus Microcella.

8. The method of claim 7, wherein the co-culturing microorganism is selected from M alkahphile, and M putealis.

9. The method of claim 1, wherein transformation of the spirulina with the transforming molecule deletes one or more genes, loci, or sequences in the spirulina genome.

10. The method of claim 1, wherein transformation of the spirulina with the transforming molecule adds one or more genes, loci, or sequences to the spirulina genome.

11. The method of claim 1, wherein transformation of the spirulina with the transforming molecule replaces one or more genes, loci, or sequences in the spirulina genome with the transforming molecule.

12. The method of claim 1, wherein the spirulina cell is transformed with multiple transforming molecules.

13. The method of claim 1, wherein the spirulina is transformed with different transforming molecules in multiple rounds of transformation.

14. The method of claim 13, wherein the spirulina is transformed with at least 2 different transforming molecules in 2 rounds of transformation.

15. The method of claim 14, wherein the first transformation inserts one transforming molecule into the spirulina genome and the second transformation replaces the first transforming molecule with a different transforming molecule inserted into the spirulina genome.

16. The method of claim 1, wherein the transforming molecule is a polynucleotide.

17. The method of claim 16, wherein the polynucleotide is DNA.

18. The method of claim 17, wherein the DNA is cDNA.

19. The method of claim 16, wherein the polynucleotide is comprised in a vector.

20. The method of claim 19, wherein the vector is a circular vector.

21. The method of claim 19, wherein the vector is linearized.

22. The method of claim 16, wherein the polynucleotide is a liner polynucleotide.

23. The method of claim 1, wherein the transforming molecule contains one or more homology arms.

24. The method of claim 23, wherein the one or more homology arms flank a sequence to be inserted into the spirulina genome.

25. The method of claim 23 or 24, wherein the homology arm is between about 1000 and about 1500 nucleotides long.

26. The method of claim 16, wherein the polynucleotide comprises one or more promoters, terminators, or enhancer sequences.

27. The method of claim 26, wherein the promoter is selected from an inducible promoter, a constitutive promoter, and a strong promoter.

28. The method of claim 1, wherein the recombinant spirulina express: (a) one or more polypeptides or fragments thereof; or (b) one or more RNA transcripts.

29. The method of claim 28, wherein the polypeptide is an antibody or fragment thereof.

30. The method of claim 29, wherein the antibody or fragment thereof is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof.

31. The method of claim 30, wherein the antibody is a VHH antibody.

32. The method of claim 28, wherein the polypeptide or fragment thereof is a therapeutic or prophylactic polypeptide.

33. The method of claim 32, wherein the therapeutic or prophylactic polypeptide is intended for delivery to the gastrointestinal tract of a subject.

34. The method of claim 32, wherein the therapeutic or prophylactic molecule is intended for systemic delivery in a subject.

35. The method of claim 32, wherein the therapeutic or prophylactic polypeptide is an endogenous spirulina polypeptide.

36. The method of claim 35, wherein the endogenous spirulina polypeptide is found in higher concentrations than found in naturally-occurring spirulina.

37. The method claim 32, wherein the therapeutic or prophylactic polypeptide is exogenous to spirulina.

38. The method of claim 37, wherein the exogenous polypeptide is naturally produced by a different bacteria or plant.

39. The method of claim 37 or 38, wherein the exogenous polypeptide is selected from the group consisting of: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin.

40. The method of claim 37 or 38, wherein the exogenous polypeptide is an antigen or epitope.

41. The method of claim 40, wherein the antigen or epitope is derived from an infectious microorganism, a tumor antigen or a self-antigen associated with an autoimmune disease

42. The method of claim 37, wherein the exogenous polypeptide or a fragment thereof is in a fusion protein.

43. The method of claim 1, wherein the spirulina is transformed with a nucleic acid, and wherein at least 2, at least 3, at least 4, or at least 5 copies of a nucleic acid sequence encoding the at least one exogenous polypeptide or fragment thereof are present in the recombinant spirulina.

44. The method of claim 1, wherein the spirulina is transformed with a nucleic acid, and wherein 2, 3, 4, 5, 6, 8, 10, 15, 20, 25, 30, 40, or 50 copies of a nucleic acid sequence encoding the at least one exogenous polypeptide or fragment thereof are present in the recombinant spirulina.

45. The method of claim 44, wherein the recombinant spirulina comprises at least 2, at least 3, at least 4, or at least 5 different exogenous polypeptides or fragments thereof.

46. The method of claim 42, wherein the fusion protein comprises a carrier protein.

47. The method of claim 46, wherein the carrier protein is selected from the group consisting of: maltose binding protein, hedgehog hepatitis virus-like particle, thioredoxin, and phycocyanin.

48. The method of claim 47, wherein the fusion protein comprises a scaffold protein.

49. The method of claim 48, wherein the at least one exogenous polypeptide is linked to a scaffold protein at the N-terminus or the C-terminus, or in the body of the scaffold protein.

50. The method of claim 48 or 49, wherein the scaffold protein is selected from the oligomerization domain of C4b-binding protein (C4BP), cholera toxin b subunit, or oligomerization domains of extracellular matrix proteins.

51. The method of claim 48, wherein the at least one exogenous polypeptide and the scaffold protein are separated by about 1 to about 50 amino acids.

52. The method of claim 42, wherein the fusion protein comprises multiple copies of the at least one exogenous polypeptide or fragment thereof, wherein the at least one exogenous polypeptide or fragment thereof and the scaffold protein are arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the at least one exogenous polypeptide or fragment thereof, SP is the scaffold protein, n, n1, and n2 represent the number of copies of the at least one exogenous polypeptide or fragment thereof.

53. The method of claim 1, wherein the therapeutic or prophylactic molecule is monomeric.

54. The method of claim 1, wherein the therapeutic or prophylactic molecule is multimeric.

55. The method of claim 1, wherein the therapeutic or prophylactic molecule is trimeric.

56. The method of claim 1, wherein the multimer is heteromeric.

57. The method of claim 1, wherein the multimer is homomeric.

58. The method of claim 1, wherein the multimer is arranged in a nanoparticle.

59. The method of claim 1, wherein the spirulina is selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

60. The method of claim 1, wherein transformation is achieved by growing the spirulina, co-culturing microorganism, and transforming molecule in liquid culture for between 1 and 4 weeks.

61. The method of claim 60, wherein the co-culture is grown in liquid culture for at least 2 weeks.

62. The method of claim 61, wherein the co-culture is grown in liquid culture for at least 3 weeks.

63. A culture of a population of recombinant spirulina cells created by the method of claim 1.

64. A recombinant spirulina cell created by the method of claim 1.

65. A method of transforming a population of spirulina cells comprising growing the spirulina cells (a) under conditions that induce competence, and (b) with a transforming molecule.

66. A composition that comprises:
a. a population of spirulina cells;
b. at least a portion of a co-culturing microorganism in an amount effective to induce competence; and
c. a transforming molecule.

67. The composition of claim 66, wherein the transforming molecule comprises a polynucleotide.

68. The composition of claim 67, wherein the polynucleotide comprises DNA.

69. The composition of claim 68, wherein the DNA is cDNA.

70. The composition of claim 69, wherein the cDNA comprises at least two sequences encoding a first and a second homology arm, and wherein the first and the second homology arm are between about 1000 and about 1500 nucleotides long.

71. The composition of claim 70, wherein the first and the second homology arm bind to a Spirulina sequence comprising at least a portion of a GNAT family N-acetyltransferase sequence.

72. The composition of claim 66, wherein the at least a portion of the co-culturing microorganism comprises the entire microorganism.

73. The composition of claim 66, wherein the at least a portion of the co-culturing microorganism comprises a portion of a microorganism.

74. The composition of claim 66, wherein at least about 5% of the spirulina cells in the population are transformed as determined by sequencing.

75. The composition of claim 70, wherein the first and the second homology arms flank a sequence encoding an antibody or fragment thereof.

76. The composition of claim 75, wherein the antibody or fragment thereof is selected from a full-length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof.

77. The composition of claim 76, comprising the VHH antibody.

78. The composition of claim 77, wherein the VHH antibody binds a target in a gastrointestinal tract.

79. The composition of claim 78, wherein the target comprises a pathogen or cancer cell.

80. The composition of claim 79, comprising the pathogen, wherein the pathogen is a bacterium.

81. The composition of claim 80, wherein the bacterium comprises campylobacter.

82. The composition of claim 66, wherein the co-culturing microorganism is a bacteria.

83. The composition of claim 82, wherein the bacteria are gram positive.

84. The composition of claim 82, wherein the bacteria are gram negative.

85. The composition of claim 82, wherein the bacteria are of an order selected from the group consisting of: Micrococcales, Xanthomonadales, Purple sulfur bacteria, Nevskiales, Hyphomicrobiales, Mycobacteriales, Bacillales, Nitrosomonadales, Oceanospirillales, Oscillatoriales, and combinations thereof.

86. The composition of claim 82, wherein the bacteria are of a genus selected from the group consisting of: Microcella, Sphingomonas, Lysobacter, Thioalkalivibrio, Luteimonas, Arenimonas, Xanthomonas, Sinimarinibacterium, Mesorhizobium, Pseudoxanthomonas, Thermomonas, Nitrosomonas, Alcanivorax, Dyella, Rhodanobacter, Halomonas, Variovorax, Frateuria, Dokdonella, Cupriavidus and Oscillatoria, Koinonema, Oxynema, Planktothrix, and Microcystis.

87. The composition of claim 86, comprising Sphingomonas or Microcella.

88. The composition of claim 66, wherein when the composition comprises a volume from about to about 40 μL, the composition comprises:
a. about 0.1 to 1 OD of the spirulina cells when measured at 750 nm wavelength as determined by spectrophotometry; and
b. about 275 ng to 325 ng of the transforming molecule.

89. A pharmaceutical generated using the method of claim 1.

90. The pharmaceutical of claim 89, wherein the pharmaceutical is in unit dose form.

91. A method of treating a disease or disorder in a subject in need thereof, comprising administering the pharmaceutical of claim 89, thereby treating the disease or disorder.

92. The method of claim 91, wherein the disease or disorder is of a tract comprising a mucosal membrane.

93. The method of claim 92, wherein the tract comprises the gastrointestinal tract.

94. The method of claim 93, wherein the disease or disorder comprises an infection of Campylobacter jejuni.

95. The method of claim 91, wherein the administering is an oral administration.

96. A container that comprises the composition of claim 66.

97. The container of claim 96, wherein the container comprises a bioreactor.

98. A kit that comprises:
(a) a composition that comprises spirulina;
(b) at least a portion of a co-culturing microorganism;
(c) a growth or storage medium; and
(d) instructions for use thereof.
